(19) United States
(12) Patent Application Publication
(10) Pub. No.: US 2024/0325417 A1
(43) Pub. Date: Oct. 3, 2024

(54) COMBINATION THERAPY FOR TREATMENT OF LIVER DISEASES

(71) Applicant: GANNEX PHARMA CO., LTD., Shanghai (CN)

(72) Inventor: Jinzi Jason WU, Shanghai (CN)

(21) Appl. No.: 18/575,325

(22) PCT Filed: Jul. 5, 2022
(86) PCT No.: PCT/CN2022/103864

§ 371 Date: Dec. 29, 2023

Related U.S. Application Data
(60) Provisional application No. 63/203,032, filed on Jul. 6, 2021.

(51) Int. Cl. A61K 31/665; A61K 31/428; A61K 31/4439; A61K 31/454; A61P 1/16

(52) U.S. Cl. CPC A61K 31/665; A61K 31/428; A61K 31/4439; A61K 31/454; A61P 1/16

(57) ABSTRACT

A pharmaceutical composition comprising: a compound of formula (I) formulated for storage at room temperature

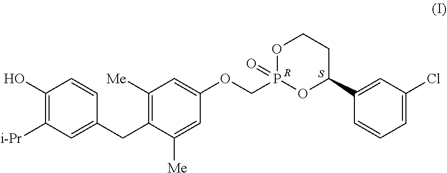

and at least one additional therapeutic agent.

### CROSS-REFERENCES TO RELATED APPLICATION

The present application is a National Stage of International Patent Application No: PCT/CN2022/103864 filed on 5 Jul. 2022, which claims the benefit of the priority of the American patent application with the application No. U.S. 63/203,032, filed on 6 Jul. 2021, the entire content of which is incorporated in this application by reference.

### FIELD

The present application generally relates to pharmaceutical compositions and medical treatments, and in particular to pharmaceutical compositions and medical treatments of liver diseases, such as non-alcoholic fatty liver disease (NAFLD) and non-alcoholic steatohepatitis (NASH).

### BACKGROUND

Nonalcoholic fatty liver disease (NAFLD) is the build up of extra fat in liver cells that is not caused by alcohol. It is normal for the liver to contain some fat. However, if more than 5%-10% percent of the liver's weight is fat, then it is called a fatty liver (steatosis). Nonalcoholic fatty liver disease (NAFLD) can be classified histologically into nonalcoholic fatty liver or nonalcoholic steatohepatitis (NASH). The worldwide prevalence of NAFLD is around 25%, and that of NASH ranges from 1.5% to 6.45%. Nonalcoholic fatty liver disease (NAFLD) is a clinicopathological term that encompasses a disease spectrum ranging from simple triglyceride accumulation in hepatocytes to hepatic steatosis with inflammation (nonalcoholic steatohepatitis, NASH) to fibrosis and cirrhosis. Hepatic insulin resistance is associated with steatosis.

The more severe form of NAFLD is called nonalcoholic steatohepatitis (NASH). NASH causes the liver to swell and become damaged. An increase in liver triglycerides can lead to increased oxidative stress in the hepatocytes, and the progression of hepatic steatosis to NASH. Oxidative stress results from an imbalance between pro-oxidant and antioxidant chemical species that leads to oxidative damage. Oxidation of fatty acids is an important source of reactive oxygen species (ROS). Some of the consequences of increased ROS is depleted ATP, destruction of membranes via lipid peroxidation, and release of proinflammatory cytokines. An increase in liver triglycerides may lead to increased oxidative stress in the hepatocytes, and the progression of hepatic steatosis to NASH. Human livers with NASH have increased lipid peroxidation and impaired mitochondrial function. This can result in cell death, hepatic stellate cell activation and fibrosis and inflammation. All of these activities may cause patients with NAFLD to be at risk for NASH, a more serious disease with higher risk of liver cirrhosis and hepatocellular carcinoma.

There is a continued need for effective treatments of NAFLD, and in particular NASH. The compositions, methods, and kits described herein address this need.

### SUMMARY

One aspect of the present application relates to a pharmaceutical composition comprising the compound of formula (I):

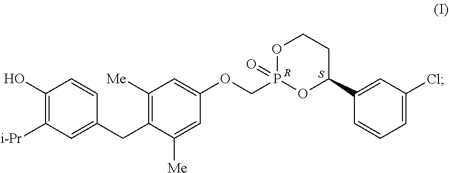

and one or more additional therapeutic agents,
wherein the compound of formula (I) is formulated for storage at room temperature.

In some embodiments, the compound of formula (I) is formulated in a stabilizing formulation that allows the compound to be stored at room temperature for at least six months.

In some embodiments, the pharmaceutical composition comprises the compound of formula (I) and the one or more additional therapeutic agents in a synergistically effective amount. In some embodiments, the pharmaceutical composition comprises a pharmaceutically acceptable carrier.

In some embodiments, the one or more additional therapeutic agents comprise a compound of formula (II)

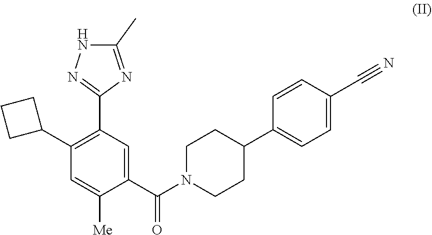 [SMILES: Cc1nc(-c2cc(C(=O)N3CCC(c4ccc(C#N)cc4)CC3)c(C)cc2C2CCC2)n[nH]1]

In some embodiments, the one or more additional therapeutic agents comprise a compound of formula (III) or capsule.

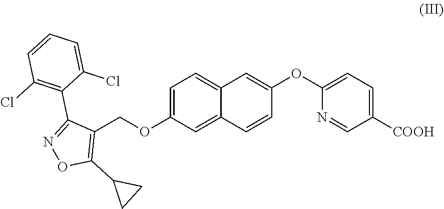 [SMILES: O=C(O)c1ccc(Oc2ccc3cc(OCc4c(-c5c(Cl)cccc5Cl)noc4C4CC4)ccc3c2)nc1]

In some embodiments, the one or more additional therapeutic agents comprise a sodium salt of the compound of formula (III).

In some embodiments, the one or more additional therapeutic agents comprise a peroxisome proliferator-activated receptor (PPAR) agonist.

Another aspect of the present application relates to a method for treatment of disease in a subject. The method comprises the step of administering to the subject (1) the compound of formula (I)

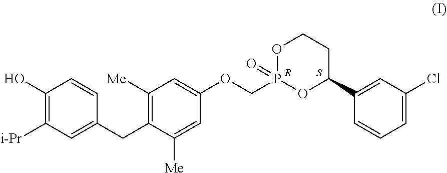

and (2) one or more additional therapeutic agents, wherein the compound of formula (I) is formulated for storage at room temperature.

In some embodiments, the compound of formula (I) and the one or more additional therapeutic agents are administered in a synergistically effective amount. In some embodiments, the one or more additional therapeutic agent comprises a compound of formula (II).

A particular embodiment of the application is a combination therapy that administers the compound of formula (I) in a formulation that is stable at room temperature

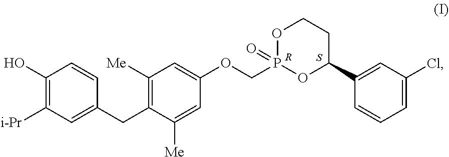

and an additional therapeutic agent, where the additional therapeutic agent is a compound of formula (II):

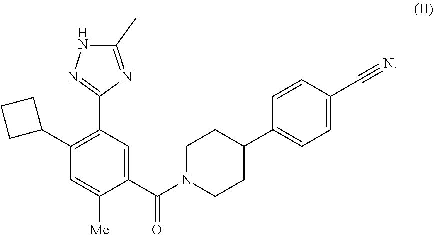 [SMILES: Cc1nc(-c2cc(C(=O)N3CCC(c4ccc(C#N)cc4)CC3)c(C)cc2C2CCC2)n[nH]1]

In some embodiments, the compounds of formula (I) and formula (II) are administered together in a fixed dose tablet or capsule. In some embodiments, the fixed dose tablet or capsul contains 1-25 mg of the compound of formula (I) and 25-300 mg of the compound of formula (II). In some embodiments, the fixed dose tablet or capsul contains 5-15 mg of the compound of formula (I) and 25-250 mg of the compound of formula (II).

In some embodiments, the fixed dose tablet or capsul contains 2.5 mg of the compound of formula (I) and 50 mg of the compound of formula (II). In some embodiments, the fixed dose tablet or capsul contains 2.5 mg of the compound of formula (I) and 75 mg of the compound of formula (II). In some embodiments, the fixed dose tablet or capsul contains 5 mg of the compound of formula (I) and 50 mg of the compound of formula (II In some embodiments, the fixed dose tablet or capsul contains 5 mg of the compound of formula (I) and 75 mg of the compound of formula (II).

Another particular embodiment of the application is a combination therapy that administers the compound of formula (I) in a formulation that is stable at room temperature

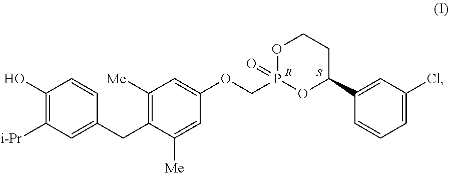

and an additional therapeutic agent, where the additional therapeutic agent is a compound of formula (III) or a salt thereof:

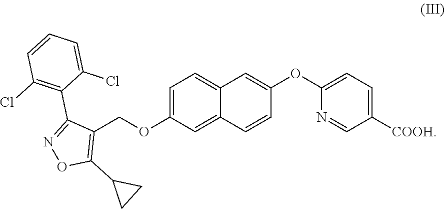 [SMILES: O=C(O)c1ccc(Oc2ccc3cc(OCc4c(-c5c(Cl)cccc5Cl)noc4C4CC4)ccc3c2)nc1]

In a further embodiment, the salt is a sodium salt of the compound of formula (III).

In some embodiments, the compounds of formula (I) and formula (III) are administered together in a fixed dose tablet or capsule. In some embodiments, the fixed dose tablet or capsul contains 1-25 mg of the compound of formula (I) and 5-100 mg of the compound of formula (III). In some embodiments, the fixed dose tablet or capsul contains 2.5-10 mg of the compound of formula (I) and 5-60 mg of the compound of formula (III). In a further embodiments, the compound of formula (III) is administered as a sodium salt.

In some embodiments, the fixed dose tablet or capsul contains 2.5 mg of the compound of formula (I) and 10 mg of the compound of formula (III). In some embodiments, the fixed dose tablet or capsul contains 2.5 mg of the compound of formula (I) and 20 mg of the compound of formula (III). In some embodiments, the fixed dose tablet or capsul contains 2.5 mg of the compound of formula (I) and 30 mg of the compound of formula (III). In some embodiments, the fixed dose tablet or capsul contains 5 mg of the compound of formula (I) and 10 mg of the compound of formula (III). In some embodiments, the fixed dose tablet or capsul contains 5 mg of the compound of formula (I) and 20 mg of the compound of formula (III). In some embodiments, the fixed dose tablet or capsul contains 5 mg of the compound of formula (I) and 30 mg of the compound of formula (III). In a further embodiments, the compound of formula (III) is administered as a sodium salt.

Another particular embodiment of the application is a combination therapy that administers the compound of formula (I) in a formulation that is stable at room temperature:

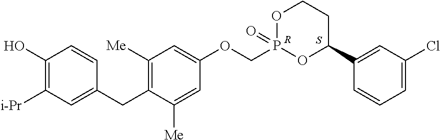

and an additional therapeutic agent, where the additional therapeutic agent is a peroxisome proliferator-activated receptor (PPAR) agonist.

In a particular embodiment, the fixed dose tablet contains 2.5 mg of the compound of formula (I) and lanifibranor. In a particular embodiment, the fixed dose tablet contains 5 mg of the compound of formula (I) and lanifibranor

In certain embodiments, the PPAR agonist is selected from one or more of the group comprising thiazolidinediones, glitazones, rosiglitazone, troglitazone, pioglitazone, englitazone, balaglitazone, rivoglitazone, ciglitazone, lobeglitazone, netoglitazone, GW 9578, GW 7647, GW 590735, GFT505, PPAR-alpha (PPAR-α) agonists, PPAR-gamma (PPAR-7) agonists, PPAR-epsilon (PPAR-6) agonists, dual PPAR-α/γ agonists, dual PPAR-α/δ agonists, pan-PPAR agonists targeting all three PPAR isozyme (i.e., α/β/γ), bezafibrate, fenofibrate, pemafibrate, gemfibrozil, clofibrate, and omega-3 poly-unsaturated fatty acids (Q-PUFAs), Omacor, INT131, MSDC-0602K, GW501516, seladelpar, saroglitazar, elafibranor, lanifibranor, netoglitazone, GW677964, DRL-605, and GW25019, and analogues, pegylated variants, and combinations of the foregoing PPAR agonists.

In some embodiments, co-administering a synergistically effective amount provides for at least one effect selected from the group consisting of: (a) a lower dose of at least one of the compound of formula (I) and at least one additional therapeutic agent; (b) a shorter treatment schedule; and (c) reduced incidence or severity of side-effects as compared to the effect obtained by administering a compound comprising the compound of formula (I) and at least one additional therapeutic agent in the absence of the other compound.

In some embodiments of the method herein, co-administration comprises any one of: simultaneous administration, sequential administration, overlapping administration, concomitant administration, interval administration, continuous administration, contemporaneous administration or any combination thereof. In some such embodiments of the method, sequential co-administration is carried out in any order.

In some embodiments of the method, the compound of formula (I) is administered orally and at least one additional therapeutic agent is administered orally or parenterally, such as, for example, by intravenous administration, intraarterial administration, intramuscular administration, subcutaneous administration, intraosseous administration, intrahecal administration, or a combination thereof.

In some embodiments, administration of the pharmaceutical combination comprising the compound of formula (I) and the additional therapeutic agent results in the prevention, treatment, or amelioration of one or more symptoms associated with a fatty liver disease in the subject. Exemplary fatty liver diseases for treatment include, but are not limited to, simple steatosis, non-alcoholic fatty liver disease (NAFLD), non-alcoholic steatohepatitis (NASH) and any combination thereof.

In other embodiments, administration of the pharmaceutical combination comprising the compound of formula (I) and the additional therapeutic agent results in one or more characteristics reflecting changes associated with the treatment of the fatty liver disease. For example, in some embodiments, administration of the compound of formula (I) and the additional therapeutic agent results in the reduction in the amount of extracellular matrix proteins present in one or more tissues of the subject with fatty liver disease.

In other embodiments, administration of the compound of formula (I) and the additional therapeutic agent results in the reduction in the amount of collagen present in one or more tissues of a subject with fatty liver disease.

In some embodiments, administration of the compound of formula (I) and the additional therapeutic agent results in a reduction in the amount of Type I, Type la, or Type III collagen present in one or more tissues of the subject with fatty liver disease.

Another aspect of the application is the use of a pharmaceutical composition comprising a compound of formula (I) and at least one additional therapeutic agent in the manufacture of a medicament for treatment of fatty liver diseases, such as, but not limited to, simple steatosis, non-alcoholic fatty liver disease (NAFLD), non-alcoholic steatohepatitis (NASH) and any combination thereof.

### BRIEF DESCRIPTION OF THE DRAWINGS

FIG. 1 is the dissolution curve of compositions prepared according to the formulations A1 to F1 in Test Example 1 (n=6) of Example 1.

FIG. 2 is the dissolution curve of compositions prepared according to the formulations a2 to e2 in Comparison Example 1 to 3 (n=6) of Example 1.

FIG. 3 is the dissolution curve of compositions prepared according to G1-L1 formulation in Test Example 2 (n=6) of Example 1.

FIG. 4 is the dissolution curve of compositions prepared according to e2-g2 formulation in Comparative Examples 4-5 (n=6) of Example 1.

FIG. 5 is an exemplary chromatogram of the impurities detected with the method described in Example 1.

While the present disclosure will now be described in detail, and it is done so in connection with the illustrative embodiments, it is not limited by the particular embodiments illustrated in the figures and the appended claims.

### DETAILED DESCRIPTION

Reference will be made in detail to certain aspects and exemplary embodiments of the application, illustrating examples in the accompanying structures and figures. The aspects of the application will be described in conjunction with the exemplary embodiments, including methods, materials and examples, such description is non-limiting and the scope of the application is intended to encompass all equivalents, alternatives, and modifications, either generally known, or incorporated here. Unless otherwise defined, all technical and scientific terms used herein have the same meaning as commonly understood by one of ordinary skill in the art to which this application belongs. One of skill in the art will recognize many techniques and materials similar or equivalent to those described here, which could be used in the practice of the aspects and embodiments of the present application. The described aspects and embodiments of the application are not limited to the methods and materials described.

### I. Definitions

In the specification and in the claims, the terms “including” and “comprising” are open-ended terms and should be interpreted to mean “including, but not limited to . . . .” These terms encompass the more restrictive terms “consisting essentially of” and “consisting of.”

As used in this specification and the appended claims, the singular forms “a,” “an” and “the” include plural referents unless the content clearly dictates otherwise.

Ranges may be expressed herein as from “about” one particular value, and/or to “about” another particular value. When such a range is expressed, another embodiment includes from the one particular value and/or to the other particular value. Similarly, when values are expressed as approximations, by use of the antecedent “about,” it will be understood that the particular value forms another embodiment. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. It is also understood that when a value is disclosed that “less than or equal to “the value,” greater than or equal to the value” and possible ranges between values are also disclosed, as appropriately understood by the skilled artisan. For example, if the value “10” is disclosed the “less than or equal to 10” as well as “greater than or equal to 10” is also disclosed.

It must be noted that as used herein and in the appended claims, the singular forms “a,” “an,” and “the” include plural reference unless the context clearly dictates otherwise. Further, the terms “a” (or “an”), “one or more” and “at least one” can be used interchangeably herein. It is also to be noted that the terms “comprising,” “including,” “characterized by” and “having” can be used interchangeably. Further, any reactant concentrations described herein should be considered as being described on a weight to weight (w/w) basis, unless otherwise specified to the contrary (e.g., mole to mole, weight to volume (w/v), etc.).

Unless defined otherwise, all technical and scientific terms used herein have the same meanings as commonly understood by one of ordinary skill in the art to which this application belongs. All publications and patents specifically mentioned herein are incorporated by reference in their entirety for all purposes including describing and disclosing the chemicals, instruments, statistical analyses and methodologies which are reported in the publications which might be used in connection with the application. All references cited in this specification are to be taken as indicative of the level of skill in the art. Nothing herein is to be construed as an admission that the application is not entitled to antedate such disclosure by virtue of prior invention.

The term “agonist” refers to a compound capable of detectably increasing the expression or activity of a given protein or receptor. The agonist can increase expression or activity by 10%, 20%, 30%, 40%, 50%, 60%, 70%, 80%, 90%, 100% or more in comparison to a control in the absence of the agonist. In embodiments, expression or activity is 1.5-fold, 2-fold, 3-fold, 4-fold, 5-fold, 10-fold or more higher than the expression or activity in the absence of the agonist. For example, an “FXR agonist” is a compound which increases FXR activity; increased FXR activity indirectly represses synthesis of bile acid and can reduce triglyceride levels in hypertriglyceridemic subjects.

The term “antagonist” or “inhibitor” refers to a compound capable of detectably decreasing the expression or activity of a given protein or receptor. The agonist can decrease expression or activity by 10%, 20%, 30%, 40%, 50%, 60%, 70%, 80%, 90%, 100% or more in comparison to a control in the absence of the antagonist. In embodiments, expression or activity is 1.5-fold, 2-fold, 3-fold, 4-fold, 5-fold, 10-fold or more lower than the expression or activity in the absence of the antagonist.

“Subject” as used herein, means a human or a non-human mammal, including but not limited to a dog, cat, horse, donkey, mule, cow, domestic buffalo, camel, llama, alpaca, bison, yak, goat, sheep, pig, elk, deer, domestic antelope, or a non-human primate selected for treatment or therapy.

A “subject suspected of having” means a subject exhibiting one or more clinical indicators of a disease or condition.

A “subject in need thereof” means a subject identified as in need of a therapy or treatment.

A “therapeutic effect” relieves, to some extent, one or more of the symptoms of a disease or disorder, and includes curing the disease or disorder. “Curing” means that the symptoms of active disease are eliminated. However, certain long-term or permanent effects of the disease may exist even after a cure is obtained (such as extensive tissue damage).

The phrase “therapeutically effective amount” as used herein refers to an amount of a compound or a combination of compounds that ameliorates, attenuates or eliminates one or more of the symptoms of a particular disease or condition or prevents, modifies, or delays the onset of one or more of the symptoms of a particular disease or condition.

The term “synergistic” as used herein refers to a therapeutic combination which is more effective than the additive effects of the two or more single agents. A determination of a synergistic interaction between the compound and at least one additional therapeutic agent may be based on the results obtained from the assays described herein.

The term “synergistically effective amount” as used herein refers to an amount of a combination of two or more agents that results in a synergistic effect. For example, if administration of 5 g of agent A result in a 10% reduction of blood pressure, administration of 5 g of agent B result in a 10% reduction of blood pressure, and administration of 10 g of a AB combination with 5 g of A and 5 g of B results in a 30% reduction of blood pressure, the 10 g of a AB combination with a A-to-B ratio of 1 is a synergistically effective amount. On the other hand, if administration of 8 g of agent A result in a 16% reduction of blood pressure, administration of 2 g of agent B result in a 4% reduction of blood pressure, and administration of 10 g of a AB combination with 8 g of A and 2 g of B results in a 20% reduction of blood pressure, the 10 g of a AB combination with a with 8 g of A and 2 g of B is not a synergistically effective amount.

“Treat”, “treatment,” and “treating,” as used herein, refer to administering a pharmaceutical composition for prophylactic and/or therapeutic purposes. The term “prophylactic treatment” refers to treating a patient who does not yet have the relevant disease or disorder, but who is susceptible to, or otherwise at risk of, a particular disease or disorder, whereby the treatment reduces the likelihood that the patient will develop the disease or disorder. The term “therapeutic treatment” refers to administering treatment to a patient already having a disease or disorder.

“Preventing” or “prevention” refers to delaying or forestalling the onset, development or progression of a condition or disease for a period of time, including weeks, months, or years.

“Amelioration” means a lessening of severity of at least one indicator of a condition or disease. In certain embodiments, amelioration includes a delay or slowing in the progression of one or more indicators of a condition or disease. The severity of indicators may be determined by subjective or objective measures which are known to those skilled in the art.

“Modulation” means a perturbation of function or activity. In certain embodiments, modulation means an increase in gene expression. In certain embodiments, modulation means a decrease in gene expression. In certain embodiments, modulation means an increase or decrease in total serum levels of a specific protein. In certain embodiments, modulation means an increase or decrease in free serum levels of a specific protein. In certain embodiments, modulation means an increase or decrease in total serum levels of a specific non-protein factor. In certain embodiments, modulation means an increase or decrease in free serum levels of a specific non-protein factor. In certain embodiments, modulation means an increase or decrease in total bioavailability of a specific protein. In certain embodiments, modulation means an increase or decrease in total bioavailability of a specific non-protein factor.

“Administering” means providing a pharmaceutical agent or composition to a subject, and includes, but is not limited to, administering by a medical professional and self-administering.

Administration of the compounds disclosed herein or the pharmaceutically acceptable salts thereof, or the additional therapeutic agents disclosed herein can be via any of the accepted modes of administration for agents that serve similar utilities including, but not limited to, orally, subcutaneously, intravenously, intranasally, topically, transdermally, intraperitoneally, intramuscularly, intrapulmonarilly, vaginally, rectally, or intraocularly. Oral and parenteral administrations are customary in treating the indications that are the subject of the preferred embodiments.

“Parenteral administration” means administration through injection or infusion. Parenteral administration includes, but is not limited to, subcutaneous administration, intravenous administration, intramuscular administration, intraarterial administration, and intracranial administration.

“Subcutaneous administration” means administration just below the skin.

“Intravenous administration” means administration into a vein.

“Intraarterial administration” means administration into an artery.

The term “agent” includes any substance, molecule, element, compound, entity, or a combination thereof. It includes, but is not limited to, e.g., protein, polypeptide, peptide or mimetic, small organic molecule, polysaccharide, polynucleotide, and the like. It can be a natural product, a synthetic compound, or a chemical compound, or a combination of two or more substances.

“In combination” or “combination” refers to the compound of formula (I) and at least one additional therapeutic agent being substantially effective in the body at a same time. Both can be administered substantially at the same time, or both can be administered at different times but have effect on the body at the same time. For example, “in combination” includes administering the compound of formula (I) before the administration of the at least one additional therapeutic agent, and subsequently administering the at least one additional therapeutic agent while functioning of the compound of formula (I) in the body is substantially extant. In addition, “in combination” includes administering the at least one additional therapeutic agent before the administration of the compound of formula (I), and subsequently administering the compound of formula (I) while functioning of the at least one additional therapeutic agent in the body is substantially extant. When a pharmaceutical composition is described as containing the compound of formula (I) and the at least one additional therapeutic agent in combination, this term refers to both agents being concurrently present in the composition. The terms “in combination” and “combination” may further relate to the advantageous use of the compound of formula (I) and the at least one additional therapeutic agent in the absence of concomitant treatment for liver diseases such as NAFLD or NASH.

“Pharmaceutical agent” means a substance that provides a therapeutic effect when administered to a subject.

“Pharmaceutical composition” means a mixture of substances suitable for administering to an individual that includes a pharmaceutical agent. For example, a pharmaceutical composition may comprise a modified oligonucleotide and a sterile aqueous solution.

“Active pharmaceutical ingredient” means the substance in a pharmaceutical composition that provides a desired effect.

The phrase “pharmaceutically acceptable” indicates that the substance or composition must be compatible chemically and/or toxicologically, with the other ingredients comprising a formulation, and/or the mammal being treated therewith.

The phrase “pharmaceutically acceptable carrier” or “pharmaceutically acceptable excipient” includes any and all solvents, diluents, emulsifiers, binders, buffers, dispersion media, coatings, antibacterial and antifungal agents, isotonic and absorption delaying agents and the like, or any other such compound as is known by those of skill in the art to be useful in preparing pharmaceutical formulations. The use of such media and agents for pharmaceutically active substances is well known in the art. Except insofar as any conventional media or agent is incompatible with the active ingredient, its use in the therapeutic compositions is contemplated. Supplementary active ingredients can also be incorporated into the compositions. In addition, various adjuvants such as are commonly used in the art may be included. These and other such compounds are described in the literature, e.g., in the Merck Index, Merck & Company, Rahway, N.J. Considerations for the inclusion of various components in pharmaceutical compositions are described, e.g., in Gilman et al. (Eds.) (1990); Goodman and Gilman's: The Pharmacological Basis of Therapeutics, 8th Ed., Pergamon Press.

A “unit dosage form” refers to a composition containing an amount of a compound that is suitable for administration to a subject, in a single dose, according to good medical practice. However, as further described below, the preparation of a single or unit dosage form however, does not imply that the dosage form is administered once per day or once per course of therapy.

A “loading dose” refers to an initial dose of a compound which is higher than subsequent doses.

A “maintenance dose” refers to a subsequent dose that follows a loading dose, and occurs later in time than a loading dose. One of ordinary skill in the art will be aware that the dosage form or mode of administration of a maintenance dose may be different from that used for the loading dose. In any of the embodiments disclosed herein, a maintenance dose may comprise administration of the unit dosage form on any dosing schedule contemplated herein, including but not limited to, monthly or multiple times per month, biweekly or multiple times each two weeks, weekly or multiple times per week, daily or multiple times per day. It is contemplated within the present disclosure that dosing holidays may be incorporated into the dosing period of the maintenance dose. Such dosing holidays may occur immediately after the administration of the loading dose or at any time during the period of administration of the maintenance dose. As used herein, the period of administration of the maintenance dose may be referred to as the “maintenance phase” of the treatment period.

A “sub-therapeutic dose” refers to an amount of a therapeutic agent that is less than the effective amount for that agent, but when combined with an effective or sub-therapeutic amount of another agent can produce a desired result, due to, for example, synergy in the resulting efficacious effects, and/or reduced side effects. For example, FDA guidelines can suggest a specified level of dosing to treat a particular condition, and a sub-therapeutic amount would be any level that is below the FDA suggested dosing level. The sub-therapeutic amount can be about 1, 5, 10, 15, 20, 25, 30, 35, 50, 75, 90, or 95% less than the amount that is considered to be a therapeutic amount. The therapeutic amount can be assessed for individual subjects, or for groups of subjects. The group of subjects can be all potential subjects, or subjects having a particular characteristic such as age, weight, race, gender, or physical activity level.

The phrase “reduced dose” refers to a dose that is less than the total daily dose to be administered to a subject.

The phrase, “mode of administration” refers to the means by which a compound is administered to a subject. As such, the phrase encompasses the dosage form (for example, a tablet, powder, dissolved liquid, suspension, emulsion, aerosol, etc.) and the mechanism by which the dosage form is applied to the subject (for example, by injection, such as subcutaneously, intramuscularly, intraperitoneally, intravenously, or intraarterially; topically, such as by cream, lotion, or patch; orally, such as by a pill, dissolved liquid, oral suspension, buccal film, or mouth rinse; nasally, such as by a nasal aerosol, powder, or spray; or ocularly, such as by an eye drop). The “mode of administration” may further encompass the dose, dose amount, and dosing schedule by which a compound is administered to a subject. The phrase “duration of the treatment” refers to the time commencing with administration of the first dose and concluding with the administration of the final dose, such length of time being determined by one of ordinary skill in the art of treating a given disease.

The phrase “dosing holiday” refers to a period of 24 hours or more during which either no dose is administered to the subject, or a reduced dose is administered to the subject.

“Fatty liver diseases” and liver disorders include the primary fatty liver diseases, steatosis or nonalcoholic fatty liver (NAFL), non-alcoholic fatty liver disease (NAFLD), non-alcoholic steatohepatitis (NASH), and hepatocellular carcinoma (HCC). Fatty liver diseases are typically conditions wherein large vacuoles of triglyceride fat accumulate in liver cells via the process of steatosis (i.e., abnormal retention of lipids within a cell). Accumulation of fat may also be accompanied by a progressive inflammation of the liver (hepatitis), called steatohepatitis. By considering the contribution of alcohol, fatty liver disease may be termed alcoholic steatosis or non-alcoholic fatty liver disease (NAFLD).

“Nonalcoholic fatty liver disease (NAFLD)” is an umbrella term for a range of liver conditions affecting people who drink little to no alcohol. As the name implies, the main characteristic of NAFLD is too much fat stored in liver cells. NAFLD is increasingly common around the world, especially in Western nations. In the United States, it is the most common form of chronic liver disease, affecting about one-quarter of the population. Some individuals with NAFLD can develop “nonalcoholic steatohepatitis (NASH),” an aggressive form of fatty liver disease, which is marked by liver inflammation and may progress to advanced scarring (cirrhosis) and liver failure. This damage is similar to the damage caused by heavy alcohol use.

The term “degradation product amount” as used herein, refers to the amount of degradation products of the compound of formula (I) as measured by chromatography as described herein. In some embodiments, the degradation products are ASC41-A, GLC02-Z2, GLC02-Z3, GLC02-Z4, GLC02-Z6, GLC02-Z7 and GLC02-Z11 as described herein. It is an unexpected discovery disclosed herein that compounds of formula (I) have greater stability when produced by hot extrusion which results in a number of degradation products as impurities described herein.

### II. Pharmaceutical Compositions

One aspect of the present application relates to a pharmaceutical composition comprising the compound of formula (I)

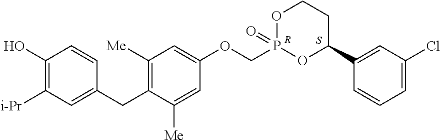

in combination with at least one additional therapeutic agent and, optionally, one or more pharmaceutically acceptable carrier(s). The IUPAC name of compound of formula (I) is (2R,4S)-4-(3-chlorophenyl)-2-[(4-{[4-hydroxy-3-(propan-2-yl)phenyl]methyl}-3,5-dimethylphenoxy)methyl]-1,3,2lambda5-dioxaphosphinan-2-one (CAS number: 852948-13-1).

In certain embodiments, the compound of formula (I) is formulated in a stabilizing formulation that allows the pharmaceutical composition to be kept at room temperature for at least 6 months. In some embodiments, the pharmaceutical composition of the present application further comprises a pharmaceutically acceptable carrier.

### (a) Stabilizing Formulation

In some embodiments, the compound of formula (I) is formulated in a stabilization formulation that allows the compound to be stored at room temperature. In some embodiments, the stabilization formulation allows the compound of formula (I) to be stored at room temperature for at least 6 month.

In some embodiments, the stabilization formulation is a hot melt extrusion product from a extrusion mixture that comprises (a) the compound of in formula (I) and (b) an extrusion medium. In some embodiment, the compound of formula (I) is in a form without solvent or crystal water. In some embodiments, the compound of formula (I) is in an amorphous form without solvent or crystal water. In some embodiments, the compound of formula (I) is in the form of a hydrate or solvate.

Examples of the extrusion medium include, but are not limited to, copovidone and hydroxypropyl methylcellulose.

In some embodiments, the extrusion mixture comprises, in weight parts, the following components: (a) 1 part of the compound of formula (I) and (b) 5 to 70 parts of copovidone with a glass transition temperature of 90° C. to 130° C.

In some embodiments, the copovidone has a glass transition temperature of 90° C. to 120° C. In some embodiments, the copovidone has a glass transition temperature of 100° C. to 120° C. In some embodiments, the copovidone has a glass transition temperature of 90° C. to 110° C. In some embodiments, the copovidone has a glass transition temperature of 100° C. to 110° C.

In some embodiments, the copovidone is common type or coarse type copovidone. In some embodiments, the copovidone is obtained by copolymerization of 1-vinyl-2-pyrrolidone and vinyl acetate in a mass ratio of 3:2, in which the nitrogen [N] content is 7.0% to 8.0% and the copolymer vinyl acetate (C₄H₆O₂) content is 35.3% to 41.4%, calculated on the anhydrous basis. The CAS number of Copovidone is 25086-89-9. Copovidone may have different names according to different naming rules or habits, such as copovidonum, poly (1-vinylpyrrolidone-vinyl acetate), polyvinylpyrrolidone-vinyl acetate copolymer, PVP/VA, PVP/VA copolymer, VP/VA copolymer 60/40, etc. The copovidone can also have different trade names according to the nomenclature of different companies, such as Kollidon® VA64 or Kollidon® VA64 fine (fine powder type) from BASF, Plasdone® S-630 from Ashland, KoVidone® VA64 from BOAI NKY MEDICAL Holdings, and Stardone® VA64 from Star-Tech & JRS Specialty Products.

In some embodiments, the weight ratio of component (a):component (b) in the extrusion mixture is 1:5-70 or 1:22-33.

In some embodiments, the extrusion mixture further comprises (c) 0.03 to 10 parts of one or more pharmaceutically acceptable excipients. In some embodiments, the one or more pharmaceutically acceptable excipients are selected from the group consisting of non-volatile weak acids, neutral and weakly acidic inorganic substances, and pharmaceutically acceptable excipients with melting point lower than 130° C., 120° C., 110° C., 100° C., 90° C. or 80° C.

Examples of non-volatile weak acids include, but are not limited to, anhydrous citric acid, citric acid monohydrate and mixtures thereof. Examples of neutral and weakly acidic inorganic substances include, but are not limited to, mannitol, lactose monohydrate, lactose anhydrous, sorbitol, calcium hydrogen phosphate anhydrous and colloidal silicon dioxide.

In some embodiments, the one or more pharmaceutically acceptable excipients comprises pharmaceutically acceptable excipients having a melting point below 80° C. In some embodiments, the pharmaceutically acceptable excipients having a melting point below 80° C. are selected from the group consisting of polyethylene glycols such as polyethylene glycol 4000 and polyethylene glycol 6000; lipidic materials such as triethyl citrate, polyethylene glycol succinate; antioxidants such as 2,6-di-tert-butyl-p-cresol and vitamin E; and surfactants such as Poloxamer 188 and Tween 8.

In some embodiments, the one or more pharmaceutically acceptable excipients have a melting point below 80° C. and are selected from the group consisting of anhydrous citric acid and citric acid monohydrate. In some embodiments, the one or more pharmaceutically acceptable excipients are selected from the group consisting of mannitol, lactose monohydrate, lactose anhydrous, sorbitol, calcium hydrogen phosphate anhydrous and colloidal silicon dioxide.

In some embodiments, the weight ratio of components (a):(b):(c) in the extrusion mixture is 1:22-33:0.03-10. In some embodiments, the weight ratio of components (a):(b):(c) in the extrusion mixture is 1:22-33:0.1-3. In some embodiments, the weight ratio of components (a):(b):(c) in the extrusion mixture is 1:22-33:0.2-2.

In some embodiments, the extrusion mixture comprises, in weight parts, the following components: (a) 1 part of the compound of Formula (I); (b) 15 to 45 parts of copovidone with a glass transition temperature of 100° C. to 120° C.; and (c) 0.1 to 3.0 parts of one or more pharmaceutically acceptable excipients selected from the group consisting of non-volatile weak acids, neutral and weakly acidic inorganic substances, and pharmaceutically acceptable excipients with melting point lower than 80° C. In some embodiments, the copovidone in (b) has a glass transition temperature of 100° C. to 110° C. In some embodiments, the drug mixture comprises 20 to 40 parts, preferably 20 to 35 parts, more preferably 22 to 33 parts of copovidone.

In some embodiments, the extrusion mixture comprises, in weight parts, the following components: (a) 1 portion of the compound of Formula (I) and (b) 3 to 40 portions of hydroxypropyl methylcellulose with a glass transition temperature of 90° C. to 130° C. The compound of Formula (I) is in a form without solvent or crystal water. In some embodiments, the compound of Formula (I) is in an amorphous form without solvent or crystal water. In some embodiments, the compound of Formula (I) is in the form of a hydrate or solvate.

In some embodiments, the hydroxypropyl methylcellulose in has a glass transition temperature of 90° C. to 120° C. In some embodiments, the hydroxypropyl methylcellulose has a glass transition temperature of 100° C. to 120° C. In some embodiments, the hydroxypropyl methylcellulose has a glass transition temperature of 90° C. to 110° C. In some embodiments, the hydroxypropyl methylcellulose has a glass transition temperature of 100° C. to 110° C. In some embodiments, the hydroxypropyl methylcellulose has a CAS number of 9004-65-3. In some embodiments, the hydroxypropyl methylcellulose suitable is the AFFINISOL® by Tao Chemical with a viscosity of 15 cP (HME15LV) or 100 cP viscosity (HME100LV).

In some embodiments, the weight ratio of components (a):(b) in the extrusion mixture is 1:9-15.

In some embodiments, the extrusion mixture further comprises (c) 0.03 to 10 parts of one or more pharmaceutically acceptable excipients. In some embodiments, the one or more pharmaceutically acceptable excipients in (c) are selected from the group consisting of non-volatile weak acids, neutral and weakly acidic inorganic substances, and pharmaceutically acceptable excipients with melting point lower than 130° C., 120° C., 110° C., 100° C., 90° C. or 80° C. Examples of non-volatile weak acids include, but are not limited to, anhydrous citric acid, citric acid monohydrate and mixtures thereof. Examples of neutral and weakly acidic inorganic substances include, but are not limited to, mannitol, lactose monohydrate, lactose anhydrous, sorbitol, calcium hydrogen phosphate anhydrous and colloidal silicon dioxide.

In some embodiments, the one or more pharmaceutically acceptable excipients have a melting point below 80° C., and are selected from the group consisting of polyethylene glycols such as polyethylene glycol 4000 and polyethylene glycol 6000; lipidic materials such as triethyl citrate, polyethylene glycol succinate; antioxidants such as 2,6-di-tert-butyl-p-cresol and vitamin E; and surfactants such as Poloxamer 188 and Tween 8.

In some embodiments, the one or more pharmaceutically acceptable excipients have a melting point below 80° C. and are selected from the group consisting of anhydrous citric acid and citric acid monohydrate. In some embodiments, the one or more pharmaceutically acceptable excipients are selected from the group consisting of mannitol, lactose monohydrate, lactose anhydrous, sorbitol, calcium hydrogen phosphate anhydrous and colloidal silicon dioxide.

In some embodiments, the weight ratio of components (a):(b):(c) in the extrusion mixture is 1:9-15:0.03-10. In some embodiments, the weight ratio of components (a):(b):(c) in the extrusion mixture is 1 1:9-15:0.1-3. In some embodiments, the weight ratio of components (a):(b):(c) in the extrusion mixture is 1:9-15:0.2-2.

In some embodiments, the extrusion mixture comprises, in weight parts, the following components: (a) 1 part of the compound of Formula (I); (b) 6 to 20 parts of hydroxypropyl methylcellulose with a glass transition temperature of 100° C. to 120° C.; and (c) 0.1 to 3.0 parts of one or more pharmaceutically acceptable excipients selected from the group consisting of non-volatile weak acids, neutral inorganic substances, weakly acidic inorganic substances, and other pharmaceutically acceptable excipients with a melting point below 80° C.

In a particular embodiment, the stabilizing formulation allows the compound of formula (I) to be at a temperature of 30° C.±2° C. and a relative humidity of 65%±5% for 6 month with a degradation product amount of less than 0.5% by weight.

### b) Additional Therapeutic Agents

In some embodiments, the pharmaceutical composition comprises the compound of formula (I) and the one or more additional therapeutic agent in a synergistically effective amount.

In some embodiments, the one or more additional therapeutic agents are selected from the group consisting of fatty acid synthase (FASN) inhibitors, farnesoid X receptor (FXR) agonists, and peroxisome proliferator-activated receptor (PPAR) agonists. In a particular embodiment, the fixed dose tablet contains 5 mg of the compound of formula (I) and lanifibranor.

In further embodiments, the one or more additional therapeutic agents are selected from the group consisting of stearoyl coenzyme A desaturase 1 (SCD1) inhibitors and fatty acid bile acid conjugates (FABAC), vitamin D receptor (VDR) agonists, glucagon-like peptide-1 (GLP-1) analogs and GLP-1 receptor agonists, acetyl-coA carboxylase (ACC) inhibitors, adenosine A3 receptor agonists, aldosterone antagonists and mineralocorticoid antagonists, AMP activated protein kinase stimulator, amylin receptor agonist and Calcitonin receptor agonists, angiopoietin-related protein-3 inhibitors, anti-LPS antibodies; apical sodium-codependent bile acid transporter inhibitors, bioactive lipids, cannabinoid CB1 receptor antagonists, caspase inhibitors, cathepsin inhibitors, chemokine receptors (CCR) antagonists, CCR3 chemokine modulators and eotaxin 2 ligand inhibitors, diacylglycerol-O-acyltransferase (DGAT) inhibitors, dipeptidyl peptidase IV (DPP4) inhibitors, insulin, insulin analog and insulin receptor agonists, insulin sensitizer and MCH receptor-1 antagonists, NOX (NADPH oxidase) inhibitors, extracellular matrix protein modulators, fibroblast growth factor 19 (FGF-19) receptor ligands, FGF-21 receptor ligands, galectin 3 inhibitors, gastric inhibitory peptides (GIP), GIP analogs, G-protein coupled receptor (GPCR) modulators, G-protein coupled receptor 84 antagonists, connective tissue growth factor ligand inhibitors and free fatty acid receptor 1 agonists, hedgehog cell-signaling pathway inhibitors, integrin inhibitors, ketohexokinase inhibitors, leukotriene (LT) inhibitors, phosphodiesterase (PDE) inhibitors, lipoxygenase (LO) inhibitors, lysyl oxidase homolog 2 inhibitors (LOXL2 inhibitors), macrolides, methyl CpG binding protein 2 modulators, transglutaminase inhibitors, miRNA antagonists, mitochondrial carrier family inhibitors, mitochondrial phosphate carrier protein inhibitors; monoclonal antibodies, myeloperoxidase inhibitors, mTOR modulators, NAD-dependent deacetylase sirtuin stimulator; type-5 phosphodiesterase (PDE 5) inhibitors, nicotinic acid receptor (GPR109) agonists, nuclear receptor ligands, P2Y13 protein agonists, phenylalanine hydroxylase stimulators, protease-activated receptor (PAR)-2 antagonists, protein kinase modulators, rho-associated protein kinase 2 (ROCK2) inhibitors, sodium-glucose transport (SGLT) 1 inhibitors, SGLT2 inhibitors, signal-regulating kinase 1 (ASK1) inhibitors, toll like receptor 2 (TLR-2) antagonists, TLR-4 antagonists, type I natural killer T cells inhibitors, tyrosine kinase receptor (RTK) modulators, urate anion exchanger 1 inhibitors, xanthine oxidase inhibitors, vascular adhesion protein-1 (VAP-1) inhibitors, anti-diabetic agents, antifibrosis compounds, anti-oxidants, anti-inflammatory compounds, lipid lowering agents, fish oil and fish oil derivatives, metabolic modulators, and analogues and pegylated variants thereof.

Examples of FASN inhibitors include, but are not limited to, TVB-2640; TVB-3664; TVB-3166, TVB-3150, TVB-3199, TVB-3693BZL-101, 2-octadecynoic acid, MDX-2, Fasnall, MT-061, G28UCM, MG-28, HS-160, GSK-2194069, KD-023 and cilostazol and compounds listed below:

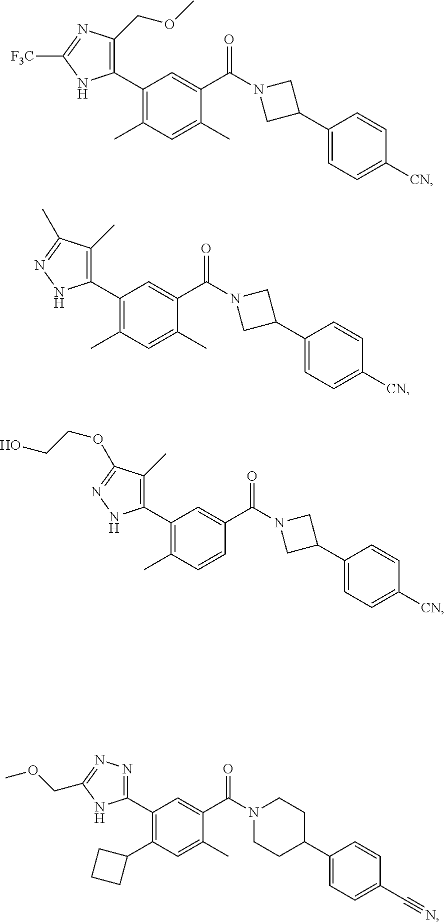 [SMILES: COCc1nc(C(F)(F)F)[nH]c1-c1cc(C(=O)N2CC(c3ccc(C#N)cc3)C2)c(C)cc1C ; Cc1cc(C)c(-c2[nH]nc(C)c2C)cc1C(=O)N1CC(c2ccc(C#N)cc2)C1 ; Cc1ccc(C(=O)N2CC(c3ccc(C#N)cc3)C2)cc1-c1[nH]nc(OCCO)c1C ; COCc1nnc(-c2cc(C(=O)N3CCC(c4ccc(C#N)cc4)CC3)c(C)cc2C2CCC2)[nH]1]

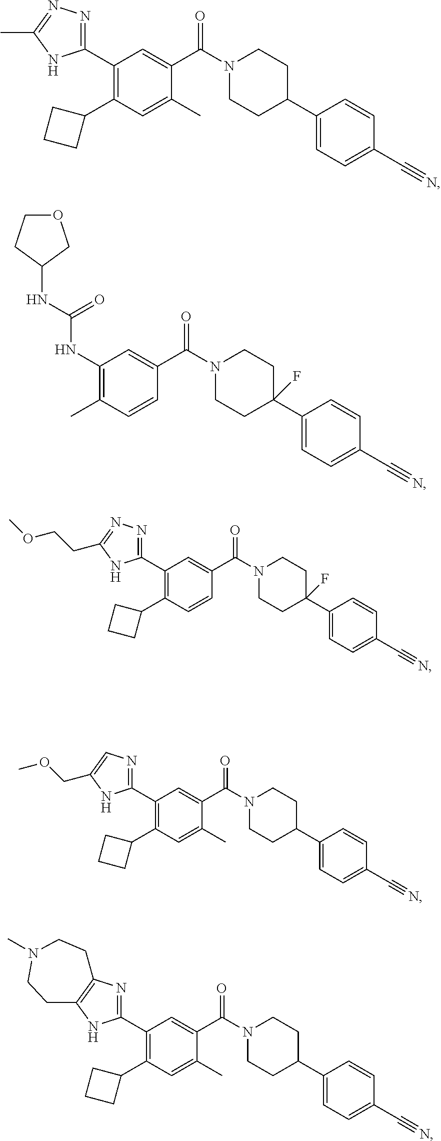 [SMILES: Cc1nnc(-c2cc(C(=O)N3CCC(c4ccc(C#N)cc4)CC3)c(C)cc2C2CCC2)[nH]1 ; Cc1ccc(C(=O)N2CCC(F)(c3ccc(C#N)cc3)CC2)cc1NC(=O)NC1CCOC1 ; COCCc1nnc(-c2cc(C(=O)N3CCC(F)(c4ccc(C#N)cc4)CC3)ccc2C2CCC2)[nH]1 ; COCc1cnc(-c2cc(C(=O)N3CCC(c4ccc(C#N)cc4)CC3)c(C)cc2C2CCC2)[nH]1 ; Cc1cc(C2CCC2)c(-c2nc3c([nH]2)CCN(C)CC3)cc1C(=O)N1CCC(c2ccc(C#N)cc2)CC1]

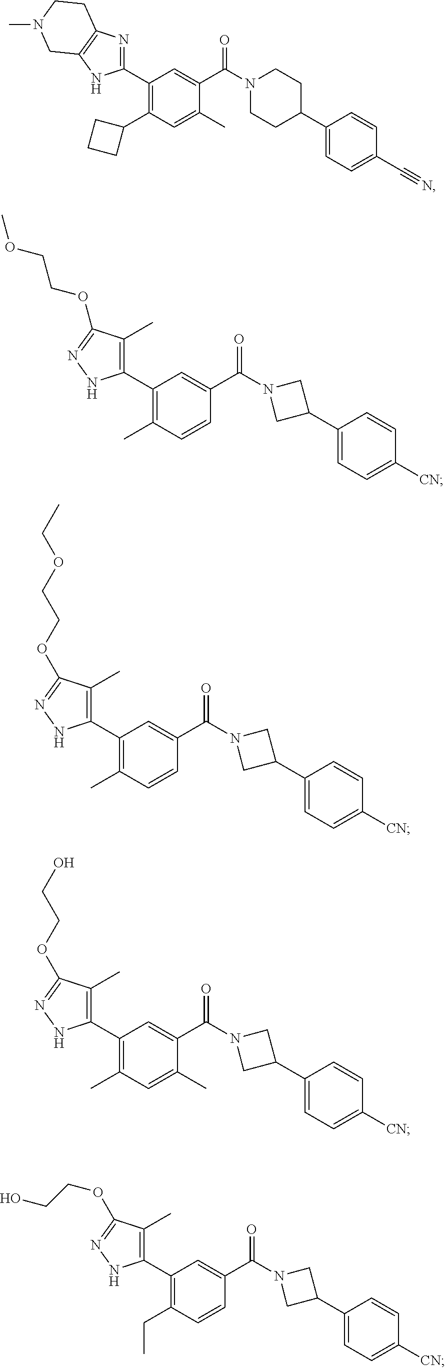 [SMILES: Cc1cc(C2CCC2)c(-c2nc3c([nH]2)CN(C)CC3)cc1C(=O)N1CCC(c2ccc(C#N)cc2)CC1 ; COCCOc1n[nH]c(-c2cc(C(=O)N3CC(c4ccc(C#N)cc4)C3)ccc2C)c1C ; CCOCCOc1n[nH]c(-c2cc(C(=O)N3CC(c4ccc(C#N)cc4)C3)ccc2C)c1C ; Cc1cc(C)c(-c2[nH]nc(OCCO)c2C)cc1C(=O)N1CC(c2ccc(C#N)cc2)C1 ; CCc1ccc(C(=O)N2CC(c3ccc(C#N)cc3)C2)cc1-c1[nH]nc(OCCO)c1C]

In some embodiments, the one or more additional therapeutic agents comprise the compound of formula (II) (which is also referred to as TVB-2640 or benzonitrile or 4-[1-[4-cyclobutyl-2-methyl-5-(3-methyl-1H-1,2,4-triazol-5-yl)benzoyl]-4-piperidinyl]).

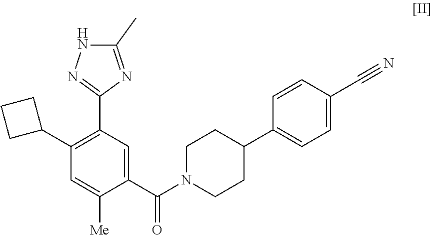 [SMILES: Cc1nc(-c2cc(C(=O)N3CCC(c4ccc(C#N)cc4)CC3)c(C)cc2C2CCC2)n[nH]1]

In some embodiments, the one or more additional therapeutic agents consist of the compound of formula (II).

The term “FXR agonist” refers to compounds that function by targeting and selectively binding FXR and which activate FXR by at least 40% above background in the assay described in Maloney et al., J. Med. Chem., Vol. 43, pp. 2971-2974 (2000). Examples of FXR agonists include, but are not limited to, INT-767, obeticholic acid (OCA), GS-9674, LJN-452 or LJN452, LMB763, EDP-305, AKN-083, INT-767, GNF-5120, LY2562175, INV-33, NTX-023-1, EP-024297, Px-103 and SR-45023. In some embodiments, the FXR agonist is a compound of formula (III)

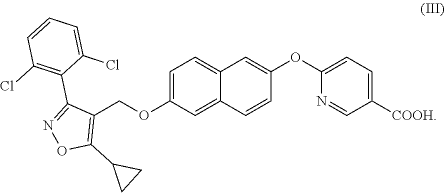 [SMILES: O=C(O)c1ccc(Oc2ccc3cc(OCc4c(-c5c(Cl)cccc5Cl)noc4C4CC4)ccc3c2)nc1]

In a further embodiment, the FXR agonist is a salt of the compound of formula (III). In a further embodiment, the FXR agonist is a sodium salt of the compound of formula (III).

Examples of PPAR agonists include, but are not limited to, elafibranor, seladepar, fenofibrate, ciprofibrate, pemafibrate, gemfibrozil, clofibrate, binifibrate, clinofibrate, clofibric acid, nicofibrate, pirifibrate, plafibride, ronifibrate, theofibrate, tocofibrate, SR10171, pioglitazone, deuterated pioglitazone, rosiglitazone, efatutazone, ATx08-001, OMS-405, CHS-131, THR-0921, SER-150-DN, KDT-501, GED-0507-34-Levo, CLC-3001, ALL-4, GW501516 (Endurabol or ({4-[({4-methyl-2-[4-(trifluoromethyl)phenyl]-1,3-thiazol-5-yl}methyl)sulfanyl]-2-methylphenoxy}acetic acid)), MBX8025 (Seladelpar or {2-methyl-4-[5-methyl-2-(4-trifluoromethyl-phenyl)-2H-[1,2,3]triazol-4-ylmethylsylfanyl]-phenoxy}-acetic acid), GW0742 ([4-[[[2-[3-fluoro-4-(trifluoromethyl)phenyl]-4-methyl-5-thiazolyl]methyl]thio]-2-methyl phenoxy]acetic acid), L165041, HPP-593, NCP-1046, saroglitazar, aleglitazar, muraglitazar, tesaglitazar, DSP-8658, T913659, conjugated linoleic acid (CLA), T3D-959, IVA337 (Lanifibranor), TTA (tetradecylthioacetic acid), Bavachinin, GW4148, GW9135, GW 9578, GW 7647, GW 590735, GFT505, INT131, MSDC-0602K, GW677964, DRL-605, GW25019, Bezafibrate, Lobeglitazone, CS038, thiazolidinediones and glitazones, e.g., rosiglitazone, troglitazone, pioglitazone, englitazone, balaglitazone, rivoglitazone, ciglitazone, lobeglitazone, and netoglitazone.

Examples of SCD1 inhibitors and FABAC include, but are not limited to, aramchol.

Examples of VDR agonists include, but are not limited to, vitamin D precursors (prodrugs), vitamin D, vitamin D analogs and active metabolites thereof that induce ligand-mediated VDR activation in vivo, such as calciferol, alfacalcidol, 1,25-dihydroxyvitamin D3, Vitamin D2, Vitamin D3, calcitriol, Vitamin D4, Vitamin D5, dihydrotachysterol, calcipotriol, tacalcitol 1,24-dihydroxyvitamin D3 and paricalcitol.

Examples of GLP-1 analogs and GLP-1 receptor agonists include, but are not limited to, albiglutide, dulaglutide, efpeglenatide, exenatide/exendin-4, taspoglutide, lixisenatide, liraglutide, lixisenatide, loxenatide, semaglutide, BRX-0585, CJC-1134-PC (exendin-4 conjugated to human albumin), LY3298176, LY-3305677, MKC-253, DLP-205, ORMD-0901, and oxyntomodulin.

Examples of acetyl-coA carboxylase (ACC) inhibitors include, but are not limited to, GS-0976, ND-654, AC-8632, PF05221304, CP640186, Gemcabene, MK-4074 and PF05175157.

Examples of adenosine A3 receptor agonists include, but are not limited to, 2-(1-Hexynyl)-N-methyladenosine, piclidenoson CF-101 (IB-MECA), namodenoson CF-102, 2-CI-IB-MECA, CP-532,903, inosine, LUF-6000, and MRS-3558.

Examples of aldosterone antagonists and mineralocorticoid receptor antagonists include, but are not limited to, apararenone (MT 3995), amiloride, spironolactone, eplerenone, canrenone and potassium canrenoate, progesterone, drospirenone, gestodene, and benidipine.

Examples of AMP activated protein kinase stimulators include, but are not limited to, PXL-770, MB-1 1055 Debio-0930B metformin, CNX-012, O-304, mangiferin calcium salt, eltrombopag, carotuximab, and Imeglimin.

Examples of amylin receptor agonist and calcitonin receptor agonists include, but are not limited to, KBP-042 and KBP-089.

Examples of angiopoietin-related protein-3 inhibitors include, but are not limited to, ARO-ANG3, IONIS-ANGGPTL3-LRx or AKCEA-ANGPTL3LRx, evinacumab, and ALN-ANG.

Examples of apical sodium-codependent bile acid transporter inhibitor include, but are not limited to, A-4250, volixibat, maralixibat formerly SHP-625, GSK-2330672, elobixibat and CJ-14199.

Examples of bile acids include, but are not limited to obeticholic acid (OCA) and UDCA, norursodeoxycholic acid, and ursodiol.

Examples of bioactive lipids include, but are not limited to 5-hydroxyeicosapentaenoic acid (15-HEPE, DS-102), unsaturated fatty acids such as 25 arachidonic acid, icosapentethyl ester, eicosapentaneoic acid, and docosahexaenoic acid.

Examples of cannabinoid CB1 receptor antagonists include, but are not limited to, namacizumab, GRC-10801, MRI-1569, MRI-1867, DBPR-211, AM-6527: AM-6545, NESS-1 1-SM, CXB-029, GCC-2680, TM-38837, Org-50189, PF-514273, BMS-812204, ZYO-1, AZD-2207, AZD-1 175, otenabant, ibipinabant, surinabant, rimonabant, drinabant, SLV-326, V-24343, and 0-2093.

Examples of caspase inhibitors include, but are not limited to, emricasan, belnacasan, nivocasan, IDN-7314, F-573, VX-166, YJP-60107, MX-1 122, IDN-6734, TLC-144, SB-234470, IDN-1965, VX-799, SDZ-220-976, and L-709049.

Examples of cathepsin inhibitors include, but are not limited to, VBY-376, VBY-825, VBY-036, VBY-129, VBY-285, Org-219517, LY3000328, RG-7236, and BF/PC-18.

Examples of CCR antagonists include, but are not limited to, CCR2/5 antagonists such as cenicriviroc; PG-092, RAP-310, INCB-10820, RAP-103, PF-04634817, and CCX-872.

Examples of CCR3 chemokine modulators and eotaxin 2 ligand inhibitors include, but are not limited to, bertilimumab, CM-101 (humanized), CM-102, and RNS-60.

Examples of DGAT inhibitors include, but are not limited to, IONIS-DGAT2RX (formerly ISIS-DGAT2Rx), LY-3202328, BH-03004, KR-69530, OT-13540, AZD-7687, PF-06865571, PF-06424439, and ABT-046.

Examples of dipeptidyl peptidase IV inhibitors include, but are not limited to evogliptin, vidagliptin, fotagliptin, alogliptin, saxagliptin, tilogliptin, anagliptin, sitagliptin, retagliptin, melogliptin, gosogliptin, trelagliptin, teneligliptin, dutogliptin, linagliptin, gemigliptin, yogliptin, betagliptin, imigliptin, omarigliptin, vidagliptin, and denagliptin.

Examples of insulin, insulin analog, and insulin receptor agonists include, but are not limited to, Humulin® R, insulin lispro (Humalog®), insulin aspart (Novolog®), insulin glulisine (Apidra®), Prompt insulin zinc (Semilente®), insulin glargine (Lantus®), insulin detemir (Levemir®), Isophane insulin, insulin zinc (Lente®), extended insulin zinc (Ultralente®), insulin degludec, Exubera®, and Afrezza®.

Examples of insulin sensitizers and MCH receptor antagonists include but are not limited to MSDC-0602k, MSDC-0602, CSTI-100 and AMRI.

Examples of NADPH oxidase (NOX) inhibitors include, but are not limited to, AS2870, VAS3947, phenothiazine derivatives, perhexiline, plumbagin, ML090, 3-methyl-1-phenyl-2-pyrazoline, imipramine, GSK2795039, GKT137831 (setanaxib), and peptide tat-gp91ds.

Examples of extracellular matrix protein modulators include, but are not limited to CNX-024, CNX-025 and SB-030.

Examples of Fractalkine ligand inhibitors include, but are not limited to, E-601 1 and KAN-0440567.

Examples of FGF-19 receptor ligand include, but are not limited to NGM-282.

Examples of FGF-21 receptor ligand include, but are not limited to, PEG-FGF21 (formerly BMS-986036), YH-25348, BMS-986171, YH-25723, LY-3025876 and NNC-0194-0499.

Examples of galectin 3 inhibitors include, but are not limited to, GR-MD-02, TD-139, ANG-4021, Galectin-3C, LJPC-201, TFD-100, GR-MD-03, GR-MD-04, GM-MD-01, GM-CT-01, GM-CT-02, Gal-100 and Gal-200.

Examples of G-protein coupled receptor (GPCR) modulators include, but are not limited to, CNX-023.

Examples of G-protein coupled receptor 84 antagonist (GPR84 antagonist), connective tissue growth factor ligand inhibitor and Free fatty acid receptor 1 agonist (FFAR1 agonist) include, but are not limited to, PBI-4050, PBI-4265, PBI-4283, and PBI-4299.

Examples of Hedgehog cell-signalling pathway inhibitors include, but are not limited to, Vismodegib, TAK-441, IPI-926, Saridegib, Sonidegib/Erismodegib, BMS-833923/XL139, PF-04449913, Taladegib/LY2940680, ETS-2400, SHR-1539, and CUR61414.

Examples of ileal sodium bile acid cotransporter inhibitors include, but are not limited to, A-4250, GSK-2330672, volixibat, CJ-14199, and elobixibat.

Examples of immunomodulators include, but are not limited to PBI-4050, PBI-4265, PBI-4283, PBI-4299 and AIC-649.

Examples of integrin inhibitors include, but are not limited to, ProAgio, and GSK-3008348.

Examples of ketohexokinase inhibitors include, but are not limited to, JNJ-28165722; JNJ-42065426; JNJ-42152981; JNJ-42740815; JNJ-42740828, and PF-06835919.

Examples of leukotriene/phosphodiesterase/lipoxygenase inhibitors include, but are not limited to tipelukast (formerly MN-001), tomelukast, sulukast, masilukast, zafirlukast, pranlukast, montelukast, gemilukast, verlukast, aklukast, pobilikast, cinalukast, and iralukast.

Examples of Lysyl oxidase homolog 2 inhibitors include, but are not limited to, Rappaport, InterMune, Pharmaxis, AB-0023, Simtuzumab, PXS-5382A, and PXS-5338.

Examples of macrolides include, but are not limited to, solithromycin, azithromycin, and erythromycin.

Examples of macrophage mannose receptor modulators include, but are not limited to AB-0023, MT-1001, [18F]FB18mHSA, Xemys, technetium Tc 99m tilmanocept, and CDX-1307.

Examples of methyl CpG binding protein 2 modulator and transglutaminase inhibitors include, but are not limited to, cysteamine, EC Cysteamine, enteric-coated cysteamine bitartrate, cysteamine bitartrate (enteric-coated), Bennu, cysteamine bitartrate (enteric-coated), Raptor, cysteamine bitartrate, DR Cysteamine, delayed release enteric coated cysteamine bitartrate, mercaptamine, mercaptamine (enteric-coated), Bennu, mercaptamine (enteric-coated), Raptor, RP-103, RP-104, PROCYSBI, and mercaptamine (enteric-coated).

Examples of miRNA antagonists include, but are not limited to, RG-125 (formerly AZD4076), RGLS-5040, RG-101, MGN-5804, and MRG-201.

Examples of metalloprotease-9 (MMP-9) stimulators include, but are not limited to, MMP-9 stimulator of Elastomics Ab.

Examples of mitochondrial carrier family inhibitor and Mitochondrial phosphate carrier protein inhibitor include, but are not limited to, TRO-19622, Trophos, olesoxime, RG-6083, or RO-7090919.

Examples of myeloperoxidase inhibitors include, but are not limited to PF-06667272.

Examples of monoclonal antibodies (mAbs) include, but is not limited to bertilimumab, NGM-313, IL-20 targeting mAbs, fresolimumab (antiTGF3) (formerly GC1008), timolumab formerly BTT-1023, namacizumab, omalizumab, ranibizumab, bevacizumab, lebrikizumab, epratuzumab, felvizumab, matuzumab, monalizumab, reslizumab, foralumab (NI-0401, anti-CD3), simtizumab (GS-6624) mAb against LOXL2, ustekinumab, inebilizumab, anti-IL20 antibodies, anti-TGF3 antibodies, anti-CD3 antibodies, anti-LOXL2 antibodies and anti-TNF antibodies.

Examples of mTOR modulators include, but are not limited to, MSDC-0602 and AAV gene therapy co-administered with SVP-sirolimus.

Examples of NAD-dependent deacetylase sirtuin stimulator; PDE5 inhibitor include, but are not limited to, NS-0200.

Examples of NF-kappa B inhibitors include, but are not limited to, LC-280126.

Examples of Nicotinic Acid Receptor (GPR109) Agonists include, but are not limited to, ARI-3037MO, MMF, LUF 6283, Acifran, IBC 293, MK-1903, GSK256073, MK-6892, MK-0354, SLx-4090, lomitapide, lexibulin, apabetalone, acifran, laropiprant, daporinad, anacetrapib, INCB-19602, ST-07-02, lomefloxacin, Niacin, and controlled release/laropiprant.

Examples of nuclear receptor ligands include, but are not limited to DUR-928

Examples of P2Y13 protein agonists include, but are not limited to CER-209.

Examples of PDGFR modulators include, but are not limited to BOT-501 and BOT-191.

Examples of phenylalanine hydroxylase stimulators include, but are not limited to Pegvaliase, sapropterin, AAV-PAH, CDX-61 14, sepiapterin, RMN-168, ALTU-236, ETX-101, HepaStem, rolipram, and alprostadil.

Examples of protease-activated receptor (PAR)-2 antagonists include, but are not limited to, PZ-235 and NP-003.

Examples of protein kinase modulators include, but are not limited to, CNX-014, MB-1 1055, ALF-1, mangiferin, amlexanox, GS-444217, REG-101 and valine.

Examples of Rho-associated protein kinase 2 (ROCK2) inhibitors include, but are not limited to, KD-025, TRX-101, BA-1049, LYC-53976, INS-1 17548 and RKI-1447.

Examples of signal-regulating kinase 1 (ASK1) inhibitors include, but are not limited to, selonsertib (formerly GS-4997).

Examples of sodium-glucose transport (SGLT) 1 inhibitors include, but are not limited to LX-4212/LX-421 1/sotagliflozin, SAR-439954, LIK-066 (Licoglifozin), LX-2761, GSK-161235, LP-925219, KGA-2727, SAR-7226, SAR-474832, SY-008, and AVX-3030.

Examples of sodium-glucose transport (SGLT) 2 inhibitors include, but are not limited to, remogliflozin, dapagliflozin, empagliflozin, ertugliflozin, sotagliflozin, ipragliflozin, tianaghflozin, canagliflozin, tofogliflozin, janagliflozin, bexagliflozin, luseoghflozin, sergliflozin, HEC-44616, AST-1935 and PLD-101.

Examples of stearoyl CoA desaturase-1 inhibitors/fatty acid bile acid conjugates include, but are not limited to, aramchol, GRC-9332, steamchol, TSN-2998, GSK-1940029 and XEN-801.

Examples of Toll Like Receptor 2 and 4 (TLR-2) antagonists include, but are not limited to, CI-201 also known as VB-201.

Examples of Toll Like Receptor 4 (TLR-4) antagonists include, but are not limited to naltrexone, JKB-121 also known as Nalmefene, M-62812, resatorvid, dendrophilin, CS-4771, AyuV-1, AyuV-25, NI-0101, EDA-HPVE7 and eritoran.

Examples of Type I natural killer T cells inhibitors include but are not limited to, GRI-0621. Illustrative Receptor tyrosine kinase (RTK) modulators include, but are not limited to CNX-025, KBP-7018, nintedanib and sorafenib.

Examples of urate anion exchanger 1 inhibitors and xanthine oxidase inhibitors include, but are not limited to, lesinurad, RLBN-1001, verinurad, KUX-1 151, and lesinurad+allopurinol.

Examples of vascular adhesion protein-1 (VAP-1) inhibitors include, but are not limited to, PXS-4728A.

In some embodiments, the one or more additional therapeutic agents comprise an agent that increases insulin secretion. In some embodiments, the one or more additional therapeutic agents comprise an agent that increases the sensitivity of target cells, tissues, or organs to insulin. In some embodiments, the one or more additional therapeutic agents comprise an agent that decreases the level of glucose in the blood.

In some embodiments, the one or more additional therapeutic agents comprise an inhibitor of the ATP-sensitive K+ channel in the pancreatic beta cells. In some embodiments, the one or more additional therapeutic agents comprise a sulfonylurea. In a further embodiment, the sulfonylurea is selected from tolbutamide (Orinase®), acetohexamide (Dymelor), tolazamide (Tolinase®), chlorpropamide (Diabinese®), carbutamide (Glucidoral®), metahexamide, glipizide (Glucotrol®), glyburide or glibenclamide (Micronase®), glycopyramide, gliquidone (Glurenorm), gliclazide (Uni Diamicron), glibornuride, glisoxepide, glimepiride (Amaryl®), and JB253 (Broichhagen et al., Nature Comm. 5, Article No. 5116 (2014)). In some embodiments, the one or more additional therapeutic agents comprise one or more agents selected from meglitinide, repaglinide (Prandin®), nateglinide (Starlix®), mitiglinide, and linogliride.

In some embodiments, the one or more additional therapeutic agents comprise an agonist of FFA1/GPR40 (Free Fatty acid Receptor 1). In a further embodiment, the FFA1/GPR40 agonist is fasiglifam.

In some embodiments, the one or more additional therapeutic agents comprise an inhibitor of dipeptidyl peptidase-4 (DPP-4, also known in the art as DPP-IV). In a further embodiment, the DPP-4 inhibitor is selected from vildagliptin (Galvus®), sitagliptin (Januvia®), saxagliptin (Onglyza®), linagliptin (Tradjenta®), alogliptin, septagliptin, anagliptin, gemigliptin, teneligliptin, carmegliptin, gosogliptin, dutogliptin, berberine and lupeol.

In some embodiments, the one or more additional therapeutic agents comprise a biguanide. In a further embodiment, the biguanide is selected from metformin, buformin, and phenformin.

In some embodiments, the one or more additional therapeutic agents comprise a bile acid sequestrant. In a further embodiment, the bile acid sequestrant is selected from anion exchange resin, quaternary amines (e.g., cholestyramine or colestipol), and an ileal bile acid transporter inhibitor.

In some embodiments, the one or more additional therapeutic agents comprise an agent that facilitates metabolism of glucose (e.g., phosphorylation of glucose). In one embodiment, the at least one additional therapeutic agent is a glucokinase activator. In a further embodiment, the glucokinase activator is a compound as described in WO 2000/058293.

In some embodiments, the one or more additional therapeutic agents comprise an agent that reduces glucose absorption in the intestine. In one embodiment, the at least one additional therapeutic agent is an alpha-glucosidase inhibitor. In a further embodiment, the alpha-glucosidase inhibitor is selected from miglitol (Glyset®), acarbose (Precose®), and voglibose.

In some embodiments, the one or more additional therapeutic agents comprise an agent that slows gastric emptying and/or suppresses glucagon. In one embodiment, the at least one additional therapeutic agent is an amylin or amylin analog. In a further embodiment, the amylin analog is pramlintide.

In some embodiments, the one or more additional therapeutic agents comprise a microsomal triglyceride transfer protein (MTP) inhibitor. In a further embodiment, the MTP inhibitor is selected from midaglizole, isaglidole, deriglidole, idazoxan, efaroxan, and fluparoxan.

In some embodiments, the one or more additional therapeutic agents comprise one or more fish oil derivatives, including, but are not limited to omega-3-fatty acid alkyl esters, including omega-3-fatty acid ethyl esters, such as ethyl (5Z,8Z,11Z,14Z, 17Z)-eicosa-5, 8,11,14,17-pentaenoate, ethyl (4Z,7 Z, 10Z, 13Z, 16Z, 19Z)-docosa-4,7, 10, 13, 16, 19-hexaenoate, ethyl (72, 10Z, 13Z, 16Z, 19Z)-docosapentaenoate, ethyl hexadecatrienoate, a-linolenic acid ethyl ester, ethyl (6Z,9Z,12Z,15Z)-6,9,12,15-octadecatetraenoate, ethyl eicosatrienoate, ethyl eicosatetraenoate, ethyl heneicosapentaenoate, ethyl icosapentaenoate, ethyl heneicosapentaenoate, ethyl tetracosapentaenoate, and nisinic acid ethyl ester. In other embodiments, the fish oil derivative is an omega-3-fatty acid trigylyceride.

In some embodiments, the one or more additional therapeutic agents comprise one or more anti-diabetic agents, including, but are not limited to, incretin hormone agonists, including glucagon-like peptide 1 receptor agonists (GLP-1RAs), GLP-1RAs include dulaglutide, semaglutide, exenatide, liraglutide, albiglutide, lixisenatide, semaglutide, insulin glargine, glucagon (GCG) and its agonists, and glucose-dependent insulinotropic polypeptide (GIP) agonists; dipeptidyl peptidase 4 (DPP4) inhibitors, DPP4 inhibitors include sitagliptin and vildagliptin; inhibitors of sodium glucose cotransporters 1 and/or 2 (SGLT1, SGLT2, and dual SGLT1/SGLT2 inhibitors), SGLT2 inhibitors include dapagliflozin, empagliflozin, canagliflozin, ipragliflozin, luseogliflozin, licogliflozin (LIK066; dual SGLT1/2); oral insulin, as well as dual or triple agonists thereof. An exemplary GLP-1/GCG receptor dual agonist is cotadutide (MEDI0382). Exemplary GLP-1/GIP receptor dual agonists include CT868 and trizepatide (LY3298176). An exemplary GLP-1/GCG/GIP triple agonist is HM15211. An exemplary dual GLP-1/FGF21 agonist is YH25724. Additional anti-diabetic drugs include metformin, pioglitazone, and rosiglitazone, as well as analogues, pegylated variants, and combinations of the foregoing anti-diabetic agents.

In some embodiments, the one or more additional therapeutic agents are selected from the group consisting of one or more of an anti-fibrotic drug selected from CCR2 and/or CCR5 antagonists, such as cenicriviroc (dual CCR2/CCR5 antagonist); apoptosis signal-regulating kinase 1 (ASK1) inhibitors, such as selonsertib; angiotensin receptor blockers (ARBs), such as losartan; transforming growth factor-β (TGF-β) inhibitors, such as galunisertib; fibroblast growth factor 19 (FGF19) and FGF19 analogs, such as NGM282; FGF21 and FGF21 analogs, such as pegbelfermin (BMS-986036), PF-05231023, AKR-001 and BI089-100; agonistic anti-FGFR1c/KLB antibodies, such as NGM313 (MK-3655) and BFKB8488A; Takeda G protein-coupled receptor 5 (TGR5) activators, such as INT-777; RDX8940; galectin-3 antagonists, such as belapectin (GR-MD-02) and GB1211; Hsp47 antagonists, such as ND-L02-s0201 siRNA; anti-lysyl oxidase-like 2 (LOXL-2) mAbs, such as simtuzumab; IL-11 inhibitors, as well as analogues, pegylated variants, and combinations thereof.

In some embodiments, the one or more additional therapeutic agents are selected from the group consisting of one or more of an anti-fibrotic drug selected from receptor tyrosine kinase inhibitors (RTKIs), such as nintedanib and sorafenib; angiotensin II (AT1) receptor blockers, a connective tissue growth factor (CTGF) inhibitor, or antifibrotic compound susceptible to interfere with the TGFβ- and BMP-activated pathways including activators of the latent TGFβ complex such as MMP2, MMP9, THBS1 or cell-surface integrins, TGF-β receptors type I (TGFBRI) or type II (TGFBRII) and their ligands, such as TGF-β, Activin, inhibin, Nodal, anti-Mullerian hormone, GDFs and BMPs; auxiliary co-receptors (also known as type III receptors); components of the SMAD-dependent canonical pathway, including regulatory or inhibitory SMAD proteins; members of the SMAD-independent or non-canonical pathways, including various branches of MAPK signaling, TAK1, Rho-like GTPase signaling pathways, phosphatidylinositol-3 kinase/AKT pathways, and TGF-β-induced epithelial-mesenchymal transition (EMT) processes; canonical and non-canonical Hedgehog signaling pathways, including Hh ligands; canonical and non-canonical wingless-type (wnt) and Notch signaling pathway inhibitors members, including those subject to TGF-β signaling; pirfenidone; nintedanib; a collagenase, such as Clostridium histolyticum collagenase; steroids (e.g., corticosteroids, such as prednisone); BMP9 and/or BMP10 antagonists; immune-suppressing and/or anti-inflammatory agents, such as gamma-interferon, cyclophosphamide, azathioprine, methotrexate, penicillamine, cyclosporine, colchicine, antithymocyte globulin, mycophenolate mofetil, and hydroxychloroquine; calcium channel blockers (e.g., nifedipine); para-aminobenzoic acid (PABA); dimethyl sulfoxide; pan caspase inhibitors; TGF-β signaling modifiers, such as relaxin, SMAD7, HGF, and BMP7, as well as TGF-β1, TGF-β RI, TGF-βR II, EGR-1, and CTGF inhibitors; cytokine and cytokine receptor antagonists (inhibitors of IL-1β, IL-5 IL-6, IL-13, IL-21, IL-4R, IL-13Rα1, GM-CSF, TNFα, oncostatin M, WISP-1, and PDGFs), cytokines and chemokines, such as IFN-γ, IFN-α/β, IL-12, IL-10, HGF, CXCL10, and CXCL11; chemokine antagonists, including inhibitors of CXCL1, CXCL2, CXCL12, CCL2, CCL3, CCL6, CCL17, and CCL18; chemokine receptor antagonists, including inhibitors of CCR2, CCR3, CCR5, CCR7, CXCR2, and CXCR4; TLR antagonists, including inhibitors of TLR3, TLR4, and TLR9; angiogenesis antagonists, such as VEGF-specific antibodies and adenosine deaminase replacement therapy, antihypertensive drugs, including beta blockers and inhibitors of ANG II, angiotensin converting enzyme (ACE), and aldosterone; vasoactive substances, such as ET-1 receptor antagonists and bosetan; inhibitors of enzymes that synthesize and process collagen, including inhibitors of prolyl hydroxylase; B cell antagonists, such as rituximab; integrin/adhesion molecule antagonists that block α1β1 and αvβ6 integrins, as well as inhibitors of integrin linked kinase; antibodies and small molecule inhibitors against ICAM-1 or VCAM-1; proapoptotic drugs targeting myofibroblasts; MMP inhibitors of MMP2, MMP9, or MMP12; antibodies and small molecule inhibitors against TIMP-1.

In some embodiments, the one or more additional therapeutic agents comprise antioxidants, including, but are not limited to vitamin E, glutathione (GSH), L-glutamyl-L-cysteinyl-glycine, ursodeoxycholic acid (UDCA), resveratrol, silymarin, metadoxine, as well as analogues, pegylated variants, and combinations thereof.

In some embodiments, the one or more additional therapeutic agents comprise one or more anti-inflammatory compounds, including, but are not limited to phosphodiesterase (PDE) inhibitors and/or tumor necrosis factor-alpha (TNF-α) inhibitors, such as pentoxifylline (PTX); L-carnitine; seloncertib; tipelukast; vitamin D3; G protein-coupled receptor 84 (GRP84); ursodeoxycholic acid (UDCA); vascular adhesion protein-1 (VAP-1)/semicarbazide-sensitive amine oxidase (SSAO) inhibitors, such as BI 1467335 (PXS-4728A), LJP-1586, and LJP-1207; caspase inhibitors, such as emricasan and GS-9450; toll-like receptor (TLR)-4 antagonists, such as JKB-121; nucleotide-binding and oligomerization domain (NOD)-like receptor (NLR) inhibitors, such as the NLR family pyrin domain containing 3 (NLRP3) inhibitor, MCC950; JAK/STAT inhibitors, glucocorticoids, NSAIDS, cyclophosphamide, nitrosoureas, folic acid analogs, purine analogs, pyrimidine analogs, methotrexate, azathioprine, mercaptopurine, ciclosporin, myriocin, tacrolimus, sirolimus, mycophenolic acid derivatives, fingolimod and other sphingosine-1-phosphate receptor modulators, monoclonal and/or polyclonal antibodies against such targets as proinflammatory cytokines and proinflammatory cytokine receptors, T-cell receptor, and integrins, analogues therefrom; pegylated variants thereof; and combinations thereof.

In some embodiments, the one or more additional therapeutic agents comprise one or more lipid lowering agents, including, but are not limited to, ezetimibe; HMG-CoA reductase inhibitors (statins), including lipophilic statins, such as atorvastatin, simvastatin, lovastatin and fluvastatin, and hydrophilic statins, such as rosuvastatin, pravastatin and pitavastatin; stearoyl-CoA desaturase 1 (SCD-1) inhibitors, such as ASC41; acetyl-CoA carboxylase (ACC) inhibitors, such as GS-0976, PF-05221304, PF-05175157, NDI-010976, firsocostat, ND-630 and ND-654; diacylglycerol O-acyltransferase-2 (DGAT-2) inhibitors, such as PF-06865571 and IONIS-DGAT2rx; fatty acid synthase (FAS) inhibitors, such as TVB-2640 and FT-4101.

In some embodiments, the one or more additional therapeutic agents comprise one or more of Acetyl-CoA carboxylase inhibitors; Adenosine A3 receptor agonists; Aldosterone antagonists and Mineralocorticoid antagonists; AMP activated protein kinase stimulator; Amylin receptor agonist and Calcitonin receptor agonists; Angiopoietin-related protein-3 inhibitors; Anti-LPS antibodies; Apical sodium-codependent bile acid transporter inhibitors; Betaine anhydrous or RM-003; bioactive lipids; Cannabinoid CB1 receptor antagonists; Dual cannabinoid CB1 receptor/iNOS inhibitor; Caspase inhibitors; Cathepsin inhibitors; CCR antagonists; CCR3 chemokine modulators and eotaxin 2 ligand inhibitors; Diacylglycerol-O-acyltransferase (DGAT) inhibitors; Dipeptidyl peptidase IV (DPP4) inhibitors; Insulin ligand and insulin receptor agonists; Insulin sensitizer and MCH receptor-1 antagonist; NOX (NADPH oxidase) inhibitors, such as dual NOX 1 and 4 inhibitors; Extracellular matrix protein modulators; Stearoyl CoA desaturase-1 inhibitors/fatty acid bile acid conjugates (FABAC); Fatty Acid Synthase (FAS) Inhibitors; Fibroblast Growth Factor 19 (FGF-19) receptor ligands, such as Recombinant Fibroblast Growth Factor 19 (FGF-19) protein, or functional engineered variant of the FGF-19 protein; Fibroblast Growth Factor 21 (FGF-21) receptor ligands such as Fibroblast Growth Factor 21 (FGF-21) protein, or functional engineered variant of the FGF-21 protein; Farnesoid X receptor (FXR) agonists; Galectin 3 inhibitors; Glucagon-like peptide-1 (GLP-1) analogs and GLP-1 receptor agonists; G-protein coupled receptor (GPCR) modulators; G-protein coupled receptor 84 antagonist, connective tissue growth factor ligand inhibitor and Free fatty acid receptor 1 agonists; Hedgehog cell-signalling pathway inhibitors; Integrin inhibitors; ketohexokinase inhibitors, Leukotriene (LTyPhosphodiesterase (PDEyLipoxygenase (LO) inhibitors; Lysyl oxidase homolog 2 inhibitors (LOXL2 inhibitors); Macrolides; Methyl CpG binding protein 2 modulator and Transglutaminase inhibitors; miRNA antagonists; Mitochondrial carrier family inhibitor and Mitochondrial phosphate carrier protein inhibitor; Monoclonal antibodies; Myeloperoxidase inhibitors; mTOR modulators; NAD-dependent deacetylase sirtuin stimulator; PDE 5 inhibitor; Nicotinic Acid Receptor (GPR109) Agonists; nuclear receptor ligands; P2Y13 protein agonists; Phenylalanine hydroxylase stimulators; Protease-activated receptor (PAR)-2 antagonists; Protein kinase modulators; Rho-associated protein kinase 2 (ROCK2) inhibitors; Sodium-GLucose Transport (SGLT) 1 inhibitors; Sodium-glucose transport (SGLT) 2 inhibitors; Stearoyl-CoA desaturase-1 inhibitors; signal-regulating kinase 1 (ASK1) inhibitors; thyroid receptor R (THR 3) agonists; Toll Like Receptor 2 (TLR-2) antagonists; Toll Like Receptor 4 (TLR-4) antagonists; Type I natural killer T cells inhibitors; Tyrosine kinase receptor (RTK) modulators; Urate anion exchanger 1 inhibitors and Xanthine oxidase inhibitors; Vascular adhesion protein-1 (VAP-1) inhibitors; Acetyl-CoA carboxylase inhibitors; Anti-LPS antibodies; Apical sodium-codependent bile acid transporter inhibitors; bioactive lipids; Cannabinoid CB1 receptor antagonists; Dual cannabinoid CB1 receptor/iNOS inhibitor; Caspase inhibitors; Cathepsin inhibitors; CCR antagonists; Diacylglycerol-O-acyltransferase (DGAT) inhibitors; Dipeptidyl peptidase IV (DPP4) inhibitors; NOX (NADPH oxidase) inhibitors, such as dual NOX 1 and 4 inhibitors; Extracellular matrix protein modulators; StearoyI CoA desaturase-1 inhibitors/fatty acid bile acid conjugates (FABAC); Galectin 3 inhibitors; Glucagon-like peptide-1 (GLP-1) analogs; G-protein coupled receptor (GPCR) modulators; Integrin inhibitors; Leukotriene (LT)/Phosphodiesterase (PDE)/Lipoxygenase (LO) inhibitors; Macrolides; miRNA antagonists; Monoclonal antibodies; rmTOR modulators; nuclear receptor ligands; P2Y13 protein agonists; Fibroblast Growth Factor 19 (FGF-19) receptor ligands, such as Recombinant Fibroblast Growth Factor 19 (FGF-19) protein, or functional engineered variant of the FGF-19 protein;—Fibroblast Growth Factor 21 (FGF-21) receptor ligands such as Fibroblast Growth Factor 21 (FGF-21) protein, or functional engineered variant of the FGF-21 protein;

In some embodiments, the one or more additional therapeutic agents comprise antibiotics, such as rifaximin, norflocacin and augmentin; mitochondrial-derived peptides, such as MOTS-c and CB4211; growth differentiation factor (GDF15) agonists, such as NGM395, NN-9215 and (LA-GDF15); mineral corticoid receptor antagonists, such as spironolactone, eplerenone, and apararenone (MT-3995); adipokines, such as leptin, adipoleptin, metreleptin, and osmotin; ileal bile acid transporter (IBAT)/apical sodium-dependent bile acid transporter (ASBT) inhibitors, such as A4250 and volixibat; thyroid hormone receptor-β (THRβ) agonists, such as resmetirom (MGL-3196); TNF-α inhibitors, such as infliximab and thalidomide; IL-1 receptor antagonists, such as anakinra; probiotics, such as VSL #3 and Lactobacillus rhamnosus GG; mitochondrial membrane transport protein modulators; androgen receptor modulators; estrogen receptor modulators; bicyclol; docosahexanoic acid (DHA); cysteamine bitartrate (CB); PXL065 (DRX-065); orlistat; IL-22; G-CSF; Imm-124E; Pirfenidone, nintedanib, and/or a fibroblast growth factor receptor antagonist, and/or a collagenase, such as Clostridium histolyticum collagenase analogues therefrom; pegylated variants thereof; and combinations thereof.

### (c) Unit Dose

The compositions described herein are preferably provided in a unit dosage form. As used herein, a “unit dosage form” is a composition containing an amount of a compound that is suitable for administration to a subject, in a single dose, according to good medical practice. The preparation of a single or unit dosage form however, does not imply that the dosage form is administered once per day or once per course of therapy. A unit dosage form may comprise a single daily dose or a fractional sub-dose wherein several unit dosage forms are to be administered over the course of a day in order to complete a daily dose. According to the present disclosure, a unit dosage form may be given more or less often than once daily, and may be administered more than once during a course of therapy. Such dosage forms may be administered in any manner consistent with their formulation, including orally, parenterally, and may be administered as an infusion over a period of time (e.g., from about 30 minutes to about 2-6 hours). While single administrations are specifically contemplated, the compositions administered according to the methods described herein may also be administered as a continuous infusion or via an implantable infusion pump.

In some embodiments, the unit dose for the compound of formula (I) is 1 mg, 2.5 mg, 5 mg, 7.5 mg, 10 mg, 12.5 mg, 15 mg or 20 mg. In some embodiments, the unit dose for the compound of formula (II) is 12.5 mg, 25 mg, 50 mg, 75 mg, 100 mg, 150 mg, 200 mg, 250 mg or 300 mg. In some embodiments, the unit dose for the compound of formula (III) is 2.5 mg, 5 mg, 10 mg, 20 mg, 30 mg, 45 mg, 60 mg, 75 mg or 100 mg. In some embodiments, the compound of formula (III) is present in a salt form. In some embodiments, the compound of formula (III) is present as a sodium salt.

In some embodiments, the compounds of formula (I) and formula (II) are administered together in a fixed dose tablet or capsule. In some embodiments, the fixed dose tablet or capsule contains 1-25 mg of the compound of formula (I) and 25-300 mg of the compound of formula (II). In some embodiments, the fixed dose tablet or capsule contains 5-15 mg of the compound of formula (I) and 25-150 mg of the compound of formula (II).

In some embodiments, the fixed dose tablet or capsule contains 2.5 mg of the compound of formula (I) and 12.5 mg, 25 mg, 37.5 mg, 50 mg, 62.5 mg, 75 mg, 100 mg, 150 mg, 200 mg, 250 mg or 300 mg of the compound of formula (II). In some embodiments, the fixed dose tablet or capsule contains 5 mg of the compound of formula (I) and 12.5 mg, 25 mg, 37.5 mg, 50 mg, 62.5 mg, 75 mg, 100 mg, 150 mg, 200 mg, 250 mg or 300 mg of the compound of formula (II). In some embodiments, the fixed dose tablet or capsule contains 7.5 mg of the compound of formula (I) and 12.5 mg, 25 mg, 37.5 mg, 50 mg, 62.5 mg, 75 mg, 100 mg, 150 mg, 200 mg, 250 mg or 300 mg of the compound of formula (II). In some embodiments, the fixed dose tablet or capsule contains 10 mg of the compound of formula (I) and 12.5 mg, 25 mg, 37.5 mg, 50 mg, 62.5 mg, 75 mg, 100 mg, 150 mg, 200 mg, 250 mg or 300 mg of the compound of formula (II). In some embodiments, the fixed dose tablet or capsule contains 12.5 mg of the compound of formula (I) and 12.5 mg, 25 mg, 37.5 mg, 50 mg, 62.5 mg, 75 mg, 100 mg, 150 mg, 200 mg, 250 mg or 300 mg of the compound of formula (II). In some embodiments, the fixed dose tablet or capsule contains 15 mg of the compound of formula (I) and 12.5 mg, 25 mg, 37.5 mg, 50 mg, 62.5 mg, 75 mg, 100 mg, 150 mg, 200 mg, 250 mg or 300 mg of the compound of formula (II).

In a particular embodiment, the fixed dose tablet or capsule contains 2.5 mg of the compound of formula (I) and 25 mg of the compound of formula (II). In a particular embodiment, the fixed dose tablet or capsule contains 2.5 mg of the compound of formula (I) and 50 mg of the compound of formula (II). In a particular embodiment, the fixed dose tablet or capsule contains 2.5 mg of the compound of formula (I) and 75 mg of the compound of formula (II).

In a particular embodiment, the fixed dose tablet or capsule contains 5 mg of the compound of formula (I) and 25 mg of the compound of formula (II). In a particular embodiment, the fixed dose tablet or capsule contains 5 mg of the compound of formula (I) and 50 mg of the compound of formula (II). In a particular embodiment, the fixed dose tablet or capsule contains 5 mg of the compound of formula (I) and 75 mg of the compound of formula (II).

In some embodiments, the compounds of formula (I) and formula (III) are administered together in a fixed dose tablet or capsule. In some embodiments, the fixed dose tablet or capsule contains 1-25 mg of the compound of formula (I) and 5-100 mg of the compound of formula (III). In some embodiments, the fixed dose tablet or capsule contains 2.5-10 mg of the compound of formula (I) and 5-60 mg of the compound of formula (III In some embodiments, the compound of formula (III) is present in the fixed dose tablet or capsule in a salt form. In some embodiments, the compound of formula (III) is present as a sodium salt.

In some embodiments, the fixed dose tablet or capsule contains 2.5 mg of the compound of formula (I) and 5 mg, 10 mg, 20 mg, 30 mg, 45 mg or 60 mg of the compound of formula (III). In some embodiments, the fixed dose tablet or capsule contains 5 mg of the compound of formula (I) and 5 mg, 10 mg, 20 mg, 30 mg, 45 mg or 60 mg of the compound of formula (III). In some embodiments, the fixed dose tablet or capsule contains 7.5 mg of the compound of formula (I) and 5 mg, 10 mg, 20 mg, 30 mg, 45 mg or 60 mg of the compound of formula (III). In some embodiments, the fixed dose tablet or capsule contains 10 mg of the compound of formula (I) and 5 mg, 10 mg, 20 mg, 30 mg, 45 mg or 60 mg of the compound of formula (III). In some embodiments, the fixed dose tablet or capsule contains 12.5 mg of the compound of formula (I) and 5 mg, 10 mg, 20 mg, 30 mg, 45 mg or 60 mg of the compound of formula (III). In some embodiments, the fixed dose tablet or capsule contains 15 mg of the compound of formula (I) and 5 mg, 10 mg, 20 mg, 30 mg, 45 mg or 60 mg of the compound of formula (III). In some embodiments, the compound of formula (III) is present in the fixed dose tablet or capsule in a salt form. In some embodiments, the compound of formula (III) is present as a sodium salt.

In a particular embodiment, the fixed dose tablet or capsule contains 2.5 mg of the compound of formula (I) and 10 mg of the compound of formula (III). In a particular embodiment, the fixed dose tablet or capsule contains 2.5 mg of the compound of formula (I) and 20 mg of the compound of formula (III). In a particular embodiment, the fixed dose tablet or capsule contains 2.5 mg of the compound of formula (I) and 30 mg of the compound of formula (III). In some embodiments, the compound of formula (III) is present in the fixed dose tablet or capsule in a salt form. In some embodiments, the compound of formula (III) is present as a sodium salt.

In a particular embodiment, the fixed dose tablet or capsule contains 5 mg of the compound of formula (I) and 10 mg of the compound of formula (III). In a particular embodiment, the fixed dose tablet or capsule contains 5 mg of the compound of formula (I) and 20 mg of the compound of formula (III). In a particular embodiment, the fixed dose tablet or capsule contains 5 mg of the compound of formula (I) and 30 mg of the compound of formula (III). In some embodiments, the compound of formula (III) is present in the fixed dose tablet or capsule in a salt form. In some embodiments, the compound of formula (III) is present as a sodium salt.

In some embodiments, the fixed dose tablets or capsules are produced by (1) prepare a stable formulation of the compound of formula (I) by hot melt extrusion, (2) mix granules or powders of the stable formulation with one or more additional therapeutic agents, such as the compound of formula (II), the compound of formula (III) or a PPAR agonist, to form a mixture and (3) press the mixture into fixed dose tablets or pack the mixture into fixed dose capsules.

In some embodiments, the compound of formula (I) and the one or more additional therapeutic agents are administered at dosages substantially the same as the dosages at which they are administered in the respective monotherapies. In some embodiments, the compound of formula (I) is administered at a dosage which is less than (e.g., less than 90%, less than 80%), less than 70%, less than 60%>, less than 50, less than 40%, less than 30%>, less than 20%, or less than 10%>) its monotherapy dosage. In some embodiments, the one or more additional therapeutic agents are administered at a dosage which is less than (e.g., less than 90%, less than 80%, less than 70%, less than 60%, less than 50, less than 40%, less than 30%, less than 20%, or less than 10%) its monotherapy dosage. In one aspect, both the first compound and the at least one additional therapeutic agent (e.g., additional therapeutic agents described herein) are administered at a dosage which is less than (e.g., less than 90%, less than 80%, less than 70%, less than 60%, less than 50, less than 40%, less than 30%, less than 20%, or less than 10%) their respective monotherapy dosages.

The actual unit dose of the active compounds described herein depends on the specific compound, and on the condition to be treated. In some embodiments, the dose may be from about 0.01 mg/kg to about 120 mg/kg or more of body weight, from about 0.05 mg/kg or less to about 70 mg/kg, from about 0.1 mg/kg to about 50 mg/kg of body weight, from about 1.0 mg/kg to about 10 mg/kg of body weight, from about 5.0 mg/kg to about 10 mg/kg of body weight, or from about 10.0 mg/kg to about 20.0 mg/kg of body weight.

In some embodiments, the unit dose may be less than 100 mg/kg, 90 mg/kg, 80 mg/kg, 70 mg/kg, 60 mg/kg, 50 mg/kg, 40 mg/kg, 30 mg/kg, 25 mg/kg, 20 mg/kg, 10 mg/kg, 7.5 mg/kg, 6 mg/kg, 5 mg/kg, 4 mg/kg, 3 mg/kg, 2.5 mg/kg, 1 mg/kg, 0.5 mg/kg, 0.1 mg/kg, 0.05 mg/kg or 0.005 mg/kg of body weight. In some embodiments, the actual unit dose is 0.05, 0.07, 0.1, 0.3, 1.0, 3.0, 5.0, 10.0 or 25.0 mg/kg of body weight. Thus, for administration to a 70 kg person, the dosage range would be from about 0.1 mg to 70 mg, from about 1 mg to about 50 mg, from about 0.5 mg to about 10 mg, from about 1 mg to about 10 mg, from about 2.5 mg to about 30 mg, from about 35 mg or less to about 700 mg or more, from about 7 mg to about 600 mg, from about 10 mg to about 500 mg, from about 20 mg to about 300 mg, or from about 200 mg to about 2000 mg.

In some embodiments, the actual unit dose is 2.5 mg. In some embodiments, the actual unit dose is 5 mg. In some embodiments the actual unit dose is 7.5 mg. In some embodiments the actual unit dose is 10 mg. In some embodiments the actual unit dose is 15 mg. In some embodiments the actual unit dose is 20 mg. In some embodiments, the actual unit dose is 25 mg. In some embodiments, the actual unit dose is 250 mg or less. In some embodiments, the actual unit dose is 100 mg or less. In some embodiments, the actual unit dose is 70 mg or less. In some embodiments, the actual unit does is 5 mg.

In some embodiments, the mode of administration comprises administering a loading dose followed by a maintenance dose. In some embodiments, the loading dose is 300 mg or less; 250 mg or less, 200 mg or less, 150 mg or less, or 100 mg or less. In some embodiments, the maintenance dose is 300 mg or less; 200 mg or less, 100 mg or less, 50 mg or less, 40 mg or less, 25 mg or less, 10 mg or less, 5 mg or less, or 1 mg or less.

In some embodiments the loading dose is administered over a period of one day. In some embodiments the loading dose is administered over a period of 2 days. In some embodiments the loading dose is administered over a period of 3 days. In some embodiments the loading dose is administered over a period of 4 days. In some embodiments the loading dose is administered over a period of 5, 6 or 7 days. In some embodiments, the loading dose is administered over a period of 8-14 days or fewer. In some embodiments, the loading dose is administered over a period of 14 days.

### (d) Pharmaceutically Acceptable Carriers

In some embodiments, the pharmaceutical application further comprises a pharmaceutically acceptable carrier. Examples of the pharmaceutically acceptable carriers include, but are not limited to, calcium carbonate, calcium phosphate, silica dioxide, sugars, starches, cellulose derivatives, gelatin, sodium stearyl fumarate, polymers such as polyethylene glycols, water, saline, phosphate buffered saline, dextrose, glycerol, ethanol, polyalcohols such as mannitol, sorbitol, and sodium chloride. In some embodiments, the pharmaceutical composition further comprises wetting or emulsifying agents, preservatives or buffering reagents, which enhance the shelf life or effectiveness of the therapeutic agents.

In some embodiments, the pharmaceutical composition is formulated for oral administration. In some embodiments, the pharmaceutical composition is formulated as a tablet, a capsule, a granule or a dry suspension. In some embodiments, the pharmaceutical composition is formulated as a tablet or a capsule. In some embodiments, the pharmaceutical composition is formulated as a hydroxypropyl cellulose capsule.

Exemplary substances, which can serve as pharmaceutically-acceptable carriers or components thereof, include sugars, such as lactose, glucose and sucrose; starches, such as corn starch and potato starch; cellulose and its derivatives, such as sodium carboxymethyl cellulose, ethyl cellulose, and methyl cellulose; powdered tragacanth; malt; gelatin; talc; solid lubricants, such as stearic acid and magnesium stearate; calcium sulfate; vegetable oils, such as peanut oil, cottonseed oil, sesame oil, olive oil, corn oil, and theobroma oil; polyols, such as propylene glycol, glycerine, sorbitol, mannitol, and polyethylene glycol; alginic acid; emulsifiers, such as the TWEENS; wetting agents, such as sodium lauryl sulfate; coloring agents; flavoring agents; tableting agents, stabilizers; antioxidants; preservatives; pyrogen-free water; isotonic saline; and phosphate buffer solutions.

The choice of a pharmaceutically-acceptable carrier to be used in conjunction with the subject compound is determined by the way the compound is to be administered.

### III. Methods of Treatment

Another aspect of the present application relates to a method for treatment of a liver disease or condition in a subject. The method comprises the step of administering to the subject (1) the compound of formula (I):

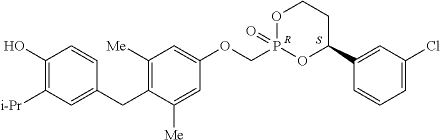

in combination with at least one additional therapeutic agent and one or more pharmaceutically acceptable carriers.

In some embodiments, the compound of formula (I) is formulated in the formulation described in Section II(a) of this application. The one of more additional therapeutic agents are described in Section II(b) of this application.

In some embodiments, the one or more additional therapeutic agents are formulated in the same pharmaceutical composition with the compound of formula (I). In some embodiments, the one or more additional therapeutic agents are formulated in a different pharmaceutical composition and administered separately. The separate administration of the one or more additional therapeutic agents may occur concurrently or sequentially with the administration of the compound of formula (I).

In some embodiments, the compound of formula (I) and the one or more additional therapeutic agents are administered in a synergistically effective amount. In some embodiments, the one or more additional therapeutic agents comprise the compound of formula (II), the compound of formula (III) or a PPAR agonist. In some embodiments, the one or more additional therapeutic agents consist of the compound of formula (II), the compound of formula (III), or a PPAR agonist.

In some embodiments, a combination therapy administers the compound of formula (I) and an additional therapeutic agent and shows therapeutic effects, where the additional therapeutic agent is a compound of formula (II), also referred to as benzonitrile, 4-[1-[4-cyclobutyl-2-methyl-5-(3-methyl-1H-1,2,4-triazol-5-yl)benzoyl]-4-piperidinyl]. In some embodiments, the compound of formula (III) is present in a salt form. In some embodiments, the compound of formula (III) is present as a sodium salt.

In some embodiments, the compounds of formula (I) and (II) are administered in a fixed dose tablet or capsule containing 2.5 mg of the compound of formula (I) and 50 mg of the compound of formula (II). In some embodiments, the compounds of formula (I) and (II) are administered in a fixed dose tablet or capsule containing 2.5 mg of the compound of formula (I) and 75 mg of the compound of formula (II). In some embodiments the compounds of formula (I) and (II) are administered in a fixed dose tablet or capsule containing 5 mg of the compound of formula (I) and 50 mg of the compound of formula (II). In some embodiments the compounds of formula (I) and (II) are administered in a fixed dose tablet or capsule containing 5 mg of the compound of formula (I) and 75 mg of the compound of formula (II).

In some embodiments, a combination therapy administers the compound of formula (I) and an additional therapeutic agent and shows therapeutic effects, where the additional therapeutic agent is the compound of formula (III). In some embodiments, the compound of formula (I) and the compound of formula (III) are administered in a fixed dose tablet or capsule. In some embodiments, the tablet or capsule contains 2.5 mg of the compound of formula (I) and 10, 20 or 30 mg of the compound of formula (III). In some embodiments, the tablet or capsule contains 5 mg of the compound of formula (I) and 10, 20 or 30 mg of the compound of formula (III). In some embodiments, the compound of formula (III) is in a salt form. In some embodiments, the compound of formula (III) is a sodium salt of formula (III).

In some embodiments, a combination therapy administers the form of compound of formula (I) and an additional therapeutic agent and shows therapeutic effects, where the additional therapeutic agent is a peroxisome proliferator-activated receptor (PPAR) agonist. In some embodiments, the compound of formula (I) and PPAR agonist are administered in a fixed dose tablet or capsule. In some embodiments, the tablet or capsule contains 2.5 mg of the compound of formula (I) and lanifibranor. In some embodiments, the tablet or capsule contains 5 mg of the compound of formula (I) and lanifibranor.

In some embodiments, the fixed dose tablets or capsules are prepared by (1) making a hot melt extrusion product of the compound of formula (I) in a stabilizing formulation, (2) mixing granules or powders of the hot melt extrusion product with one or more therapeutic agents, such as the compound of formula (II), the compound of formula (III) and/or a PPAR agonist, to produce a mixture, and (3) pressing the mixture into fixed dose tablets or packing the mixture into fixed dose capsules.

### (a) Liver Diseases and Conditions

In some embodiments, the liver diseases and conditions are THRβ-related diseases and conditions. In some embodiments, the liver diseases and conditions are FXR-related diseases and conditions. In some embodiments, the liver diseases and conditions are FASN-related diseases and conditions. In some embodiments, the liver diseases and conditions are simple steatosis, NAFLD and NASH.

In some embodiments, the liver diseases and conditions are fatty liver diseases, fibrotic disorders and inflammatory conditions affecting the liver.

In some embodiments, the liver diseases and conditions are secondary fatty liver diseases such as alcoholic liver disease (ALD), fatty liver associated with chronic hepatitis infection, total parental nutrition (TPN), Reye's Syndrome, as well as gastrointestinal disorders, such as intestinal bacterial overgrowth (IBO), gastroparesis, irritable bowel (IBS) disorders, and the like.

In some embodiments, the liver diseases and conditions are disease are liver fibrosis, such as steatosis, non-alcoholic fatty liver disease (NAFLD), non-alcoholic steatohepatitis (NASH), alcoholic steatohepatitis (ASH), and hepatocellular carcinoma (HCC).

In some embodiments, the subject for treatment has NAFLD. In some embodiments, the subject has diabetes. In some embodiments, the subject has type 2 diabetes. In some embodiments, the subject has type 1 diabetes. In certain embodiments, the subject with NAFLD has type 2 diabetes mellitus (T2DM). In other embodiments, the subject with NAFLD has metabolic syndrome (MS).

In some embodiments, the subject has a metabolic disease or disorder. Exemplary metabolic diseases or disorders for treatment with the compositions of the present application include diabetes, metabolic syndrome, obesity, hyperlipidemia, high cholesterol, arteriosclerosis, hypertension, NASH, NAFL, NAFLD, hepatic steatosis, and any combination thereof.

In some embodiments, the subject has metabolic syndrome (MS). In some embodiments, the subject has one or more of these diseases or disorders. In some embodiments, the subject is at risk of developing one or more of these diseases.

In some embodiments, the subject has insulin resistance, increased blood glucose concentrations, high blood pressure, elevated cholesterol levels, elevated triglyceride levels, or is obese.

In some embodiments, the subject has polycystic ovary syndrome.

In some embodiments, the patient being treated is at risk of developing liver fibrosis or cirrhosis.

In some embodiments, the fibrosis comprises non-cirrhotic hepatic fibrosis.

In some embodiments, the liver fibrosis is advanced.

In some embodiments, the disease effects tissue selected from the group consisting of liver, kidney, skin, epidermis, endodermis, muscle, tendon, cartilage, heart, pancreas, lung, uterus, nervous system, testis, penis, ovary, adrenal gland, artery, vein, colon, intestine (e.g. small intestine), biliary tract, soft tissue (e.g. mediastinum or retroperitoneum), bone marrow, joint and stomach fibrosis, in particular liver, gut, lung, heart, kidney, muscle, skin, soft tissue, bone marrow, intestinal, eye and joint fibrosis.

In some embodiments, the disease is selected from the group consisting of metabolic liver diseases, non-alcoholic fatty liver disease (NAFLD), non-alcoholic steatohepatitis (NASH), drug-induced liver diseases, alcohol-induced liver diseases, infectious agent induced liver diseases, inflammatory liver diseases, immune system dysfunction-mediated liver diseases, dyslipidemia, cardiovascular diseases, restenosis, syndrome X, metabolic syndrome, diabetes, obesity, hypertension, chronic cholangiopathies such as Primary Sclerosing Cholangitis (PSC), Primary Biliary Cholangitis (PBC), biliary atresia, progressive familial intrahepatic cholestasis type 3 (PFIC3), inflammatory bowel diseases, Crohn's disease, ulcerative colitis, keloid, old myocardial infarction, scleroderma/systemic sclerosis, inflammatory diseases, neurodegenerative diseases, cancers, liver cancer, hepatocallular carcinoma, gastrointestinal cancer, gastric cancer, meningioma associated with neurofibromatosis, pancreatic neuroendocrine tumors, pancreatic exocrine tumors, leukemia, myeloproliferative/myelodisplastic diseases, mastocytosis, dermatofibrosarcoma, solid tumors including breast, lung, thyroid or colorectal cancer, a prostate cancer, liver fibrosis or cirrhosis of any origin, metabolic disease-induced liver fibrosis or cirrhosis, NAFLD-induced fibrosis or cirrhosis, NASH-induced fibrosis or cirrhosis, alcohol-induced liver fibrosis or cirrhosis, drug-induced liver fibrosis or cirrhosis, infectious agent-induced liver fibrosis or cirrhosis, parasite infection-induced liver fibrosis or cirrhosis, bacterial infection-induced liver fibrosis or cirrhosis, viral infection-induced fibrosis or cirrhosis, HBV-infection induced liver fibrosis or cirrhosis, HCV-infection induced liver fibrosis or cirrhosis, HIV-infection induced liver fibrosis or cirrhosis, dual HCV and HIV-infection induced liver fibrosis or cirrhosis, radiation- or chemotherapy-induced fibrosis or cirrhosis, biliary tract fibrosis, liver fibrosis or cirrhosis due to any chronic cholestatic disease, gut fibrosis of any etiology, Crohn's disease-induced fibrosis, ulcerative colitis-induced fibrosis, intestine (e.g. small intestine) fibrosis, colon fibrosis, stomach fibrosis, skin fibrosis, epidermis fibrosis, endodermis fibrosis, skin fibrosis due to scleroderma/systemic sclerosis, lung fibrosis, lung fibrosis consecutive to chronic inflammatory airway diseases, such as COPD, asthma, emphysema, smoker's lung, tuberculosis, pulmonary fibrosis, idiopathic pulmonary fibrosis (IPF), heart fibrosis, kidney fibrosis, nephrogenic systemic fibrosis, muscle fibrosis, soft tissue (e.g. mediastinum or retroperitoneum) fibrosis, bone marrow fibrosis, joint fibrosis, tendon fibrosis, cartilage fibrosis, pancreas fibrosis, uterus fibrosis, nervous system fibrosis, testis fibrosis, ovary fibrosis, adrenal gland fibrosis, artery fibrosis, vein fibrosis, eye fibrosis, endomyocardial fibrosis, mediastinal fibrosis, myelofibrosis, retroperitoneal fibrosis, progressive massive fibrosis (a complication of coal workers' pneumoconiosis), proliferative fibrosis, neoplastic fibrosis, peri-implantational fibrosis and asbestosis, arthrofibrosis, adhesive capsulitis.

In some embodiments, the disease is selected from the group consisting of metabolic liver diseases, non-alcoholic fatty liver disease (NAFLD), non-alcoholic steatohepatitis (NASH), drug-induced liver diseases, alcohol-induced liver diseases, infectious agent induced liver diseases, inflammatory liver diseases, immune system dysfunction-mediated liver diseases, dyslipidemia, cardiovascular diseases, restenosis, syndrome X, metabolic syndrome, diabetes, obesity, hypertension, chronic cholangiopathies such as Primary Sclerosing Cholangitis (PSC), Primary Biliary Cholangitis (PBC), biliary atresia, progressive familial intrahepatic cholestasis type 3 (PFIC3), inflammatory bowel diseases, Crohn's disease, ulcerative colitis, liver cancer, hepatocallular carcinoma, gastrointestinal cancer, gastric cancer, colorectal cancer, metabolic disease-induced liver fibrosis or cirrhosis, NAFLD-induced fibrosis or cirrhosis, NASH-induced fibrosis or cirrhosis, alcohol-induced liver fibrosis or cirrhosis, drug-induced liver fibrosis or cirrhosis, infectious agent-induced liver fibrosis or cirrhosis, parasite infection-induced liver fibrosis or cirrhosis, bacterial infection-induced liver fibrosis or cirrhosis, viral infection-induced fibrosis or cirrhosis, HBV-infection induced liver fibrosis or cirrhosis, HCV-infection induced liver fibrosis or cirrhosis, HIV-infection induced liver fibrosis or cirrhosis, dual HCV and HIV-infection induced liver fibrosis or cirrhosis, radiation- or chemotherapy-induced fibrosis or cirrhosis, biliary tract fibrosis, liver fibrosis or cirrhosis due to any chronic cholestatic disease, gut fibrosis of any etiology, Crohn's disease-induced fibrosis, ulcerative colitis-induced fibrosis, intestine (e.g. small intestine) fibrosis, colon fibrosis, stomach fibrosis, lung fibrosis, lung fibrosis consecutive to chronic inflammatory airway diseases, such as COPD, asthma, emphysema, smoker's lung, tuberculosis, pulmonary fibrosis, idiopathic pulmonary fibrosis (IPF).

In some embodiments, administration of the compound of formula (I) of the present application in combination with the one or more additional therapeutic agents results in the prevention, treatment, or amelioration, of simple steatosis, NAFLD, or NASH in a subject.

In some embodiments, administration of the compound of formula (I) of the present application results in the prevention, treatment, or amelioration, of simple steatosis, NAFLD, or NASH in a subject such that the therapeutic effects accompanying the co-administration is synergistic compared to either agent alone.

In some embodiments, administration of the compound of formula (I) of the present application results in the reduction in the amount of collagen present in one or more tissues of a subject with fatty liver disease.

In some embodiments, administration of the compound of formula (I) of the present application results in a reduction in the amount of Type I, Type la, or Type III collagen present in one or more tissues of the subject with fatty liver disease.

In some embodiments, the present application also provides a method for reducing bilirubin levels in a subject. In some embodiments, the method of the present application reduces the amount of serum bilirubin in the subject by at least 10%, 20%, 30%, 40%, 50%, 60%, 70%, 80%, or 90%, as compared to a control subject (e.g., a subject not administered with the composition of the present application). In one example, the subject has an elevated level of bilirubin, as compared to a healthy subject (e.g., an individual without a disease or condition, such as those described herein). In one example, the method of the present application reduces the level of bilirubin to a normal level (e.g., similar to the level of bilirubin in an individual without a disease or condition, such as those described herein). In a further example, the method of the present application reduces the level of bilirubin below 10 mg/L, 9 mg/L, 8 mg/L, 7 mg/L, 6 mg/L, 5 mg/L, 4 mg/L, 3 mg/L, 2 mg/L, 1.5 mg/L, 1.2 mg/L, or 1 mg/L. In a further example, the method of the present application reduces the level of bilirubin below 2 mg/L, 1.5 mg/L, 1.2 mg/L, or 1 mg/L.

In some embodiments, the present application also provides a method for reducing the serum level of a lever enzyme in a subject. In some embodiments, the liver enzyme is selected from the group consisting of alkaline phosphatase (ALP, AP, or Alk Phos), alanine aminotransferase (ALT), aspartate, aminotransferase (AST), gamma-glutamyl transpeptidase (GGT), lactate dehydrogenase (LDH), and 5′ nucleotidase. In some embodiments, the method of the present application reduces the amount of one or more liver enzymes by at least 10%, 20%, 30%, 40%, 50%, 60%, 70%, 80%, or 90%, as compared to a control subject (e.g., a subject not administered with the composition of the present application). In some embodiments, the present application also provides a method for reducing bilirubin levels in a subject, the subject has elevated levels of one or more liver enzymes, as compared to a healthy subject (e.g., an individual without a disease or condition, such as those described herein).

In some embodiments, the method of the present application reduces the serum level of ALP in the subject below 500 IU/L (international units per liter), 400 IU/L, 300 IU/L, 200 IU/L, 180 IU/L, 160 IU/L, or 150 IU/L. In a further example, the method of the present application reduces the level of ALP to from about 40 IU/L to about 150 IU/L. In a further example, the method of the present application reduces the level of ALT below 200 IU/L (international units per liter), 150 IU/L, 100 IU/L, 80 IU/L, 60 IU/L, or 50 IU/L. In a further example, the method of the present application reduces the level of ALT to from about 5 IU/L to about 50 IU/L.

In some embodiments, the method of the present application reduces the level of AST in the subject below 200 IU/L (international units per liter), 150 IU/L, 100 IU/L, 80 IU/L, 60 IU/L, 50 IU/L, or 40 IU/L. In a further example, the method of the present application reduces the level of AST to from about 10 IU/L to about 50 IU/L.

In some embodiments, the method of the present application reduces the level of GGT in the subject below 200 IU/L (international units per liter), 150 IU/L, 100 IU/L, 90 IU/L, 80 IU/L, 70 IU/L, or 60 IU/L. In a further example, the method of the present application reduces the level of GGT to from about 15 IU/L to about 50 IU/L or from about 5 IU/L to about 30 IU/L.

In some embodiments, the method of the present application reduces the level of LDH in the subject below 500 IU/L (international units per liter), 400 IU/L, 300 IU/L, 200 IU/L, 180 IU/L, 160 IU/L, 150 IU/L, 140 IU/L, or 130 IU/L. In a further example, the method of the present application reduces the level of LDH to from about 120 IU/L to about 220 IU/L.

In some embodiments, the method of the present application reduces the level of 5′ nucleotidase in the subject below 50 IU/L (international units per liter), 40 IU/L, 30 IU/L, 20 IU/L, 18 IU/L, 17 IU/L, 16 IU/L, 15 IU/L, 14 IU/L, 13 IU/L, 12 IU/L, 11 IU/L, 10 IU/L, 9 IU/L, 8 IU/L, 7 IU/L, 6 IU/L, or 5 IU/L. In a further example, the method of the present application reduces the level of 5′ nucleotidase to from about 2 IU/L to about 15 IU/L.

In some embodiments, the present application also provides a method for reducing glucose levels in a subject, wherein the subject has elevated levels of glucose, as compared to a healthy subject (e.g., an individual without a disease or condition, such as those described herein). In some embodiments, the method of the present application reduces post-meal glucose levels below 800 mg/L, 700 mg/L, 600 mg/L, 500 mg/L, 400 mg/L, 350 mg/L, 300 mg/L, 250 mg/L, 240 mg/L, 230 mg/L, 220 mg/L, 210 mg/L, 200 mg/L, 190 mg/L, 180 mg/L, 170 mg/L, 160 mg/L, or 150 mg/L. In one example, the method of the present application reduces post-meal glucose levels below 200 mg/L, 190 mg/L, 180 mg/L, 170 mg/L, 160 mg/L, or 150 mg/L. In some embodiments, the method of the present application reduces fasting glucose levels to 70-800 mg/L, 70-700 mg/L, 70-600 mg/L, 70-500 mg/L, 70-400 mg/L, 70-350 mg/L, 70-300 mg/L, 70-250 mg/L, 70-240 mg/L, 70-230 mg/L, 70-220 mg/L, 70-210 mg/L, 70-200 mg/L, 70-190 mg/L, 70-180 mg/L, 70-170 mg/L, 70-160 mg/L, 70-150 mg/L, 70-140 mg/L, 70-130 mg/L, 70-120 mg/L, 70-110 mg/L, 70-100 mg/L, 90-130 mg/L, 90-120 mg/L, 90-110 mg/L, or 90-100 mg/L. In one example, the method of the present application reduces post-meal glucose levels to 70-200 mg/L, 70-190 mg/L, 70-180 mg/L, 70-170 mg/L, 70-160 mg/L, 70-150 mg/L, 70-140 mg/L, 70-130 mg/L, 70-120 mg/L, 70-110 mg/L, 70-100 mg/L, 90-130 mg/L, 90-120 mg/L, 90-110 mg/L, or 90-100 mg/L.

The present application also provides a method for reducing hemoglobin Ale (HbAlc) levels (i.e., amount of HbAlc), such as in the blood, comprising administering a therapeutically effective amount of a pharmaceutical composition of the present application to a subject in need thereof. In some embodiments, the method reduces the HbAlc levels by at least 10%>, 20%>, 30%>, 40%>, 50%), 60%), 70%), 80%), or 90%>, as compared to a control subject (e.g., a subject not administered with the composition of the present application). In one example, the subject has elevated levels of HbAlc, as compared to a healthy subject (e.g., an individual without a disease or condition, such as those described herein). In one example, the method of the present application reduces the HbAlc levels to normal levels (e.g., similar to the HbAlc levels in an individual without a disease or condition, such as those described herein).

In some embodiments, the subject has elevated levels of HbAlc, as compared to a healthy subject (e.g., an individual without a disease or condition, such as those described herein). In one example, the method of the present application reduces HbAlc levels below 10%, 9.5%, 9.0%, 8.5%, 8.0%, 7.5%, 7.0%, 6.5%, 6.4%, 6.3%, 6.2%, 6.1%, 6.0%, 5.9%, 5.8%, or 5.7%. In one example, the method of the present application reduces HbAlc levels below 8.0%, 7.9%, 7.8%, 7.7%, 7.6%, 7.5%, 7.4%, 7.3%, 7.2%, 7.1%, 7.0%, 6.9%, 6.8%, 6.7%, 6.6%, 6.5%, 6.4%, 6.3%, 6.2%, 6.1%, 6.0%, 5.9%, 5.8%, or 5.7%. In one example, the method of the present application reduces HbAlc levels below 6.5%, 6.4%, 6.3%, 6.2%, 6.1%, 6.0%, 5.9%, 5.8%, or 5.7%.

The present application also provides a method for increasing insulin secretion (i.e., amount of insulin), comprising administering a therapeutically effective amount of a pharmaceutical composition of the present application to a subject in need thereof. In some embodiments, the method of the present application increases insulin secretion by at least 10%, 20%, 30%, 40%, 50%, 60%, 70%, 80%, or 90%, as compared to a control subject (e.g., a subject not administered with the composition of the present application). In one example, the subject has decreased secretion of insulin, as compared to a healthy subject (e.g., an individual without a disease or condition, such as those described herein). In one example, the method of the present application increases insulin secretion such that the insulin level is of 2-9.0 mlU/mL, 2-8.0 mlU/mL, 2-7.0 mlU/mL, 2-6.0 mlU/mL, 3-9.0 mlU/mL, 3-8.0 mlU/mL, 3-7.0 mlU/mL, 3-6.0 mlU/mL, 4-9.0 mlU/mL, 4-8.0 mlU/mL, 4-7.0 mlU/mL, 4-6.0 mlU/mL, 5-9.0 mlU/mL, 5-8.0 mlU/mL, 5-7.0 mlU/mL or 5-6.0 mlU/mL.

The present application also provides a method for increasing insulin sensitivity (i.e., decreasing insulin resistance), comprising administering a therapeutically effective amount of a pharmaceutical composition of the present application to a subject in need thereof. In one example, the method of the present application increases insulin sensitivity (i.e., decreases insulin resistance) by at least 10%, 20%, 30%, 40%, 50%, 60%, 70%, 80%, or 90%, as compared to a control subject (e.g., a subject not administered with the composition of the present application). In some embodiments, the subject has decreased insulin sensitivity (i.e., increased insulin resistance), as compared to a healthy subject (e.g., an individual without a disease or condition, such as those described herein).

### (b) Routes, Regimen and Dosages of Administration

Administration of the active agents described herein may be achieved by modulating the dosing schedule such that subjects experience periodic partial or full reductions in dosing for fixed amounts of time, followed by a resumption of dosing.

In some embodiments, dosages are administered daily for between one and thirty days, followed by a dosing holiday lasting for between one and thirty days.

In some embodiments, during the dosing holiday, no dose is administered.

In further embodiments, the compound of formula (I) and its metabolites are allowed to clear completely from the subject's body prior to administration of the next dose.

In some other embodiments, during the dosing holiday, a dose less than the usual daily dose is administered.

In some further embodiments, an amount of the administered compound of formula (I) less than the therapeutically effective amount is allowed to remain within the subject during the dosing holiday.

In some further embodiments, an amount of the administered compound of formula (I) sufficient to maintain therapeutic levels in the affected tissues is allowed to remain within the subject.

In some embodiments, the maximum serum concentration of the compound of formula (I) during the dosing schedule is less than 120 ng/ml, less than 100 ng/ml, less than 90 ng/ml, less than 80 ng/ml, less than 70 ng/ml, less than 60 ng/ml, or less than 50 ng/ml.

In some embodiments, the minimum serum concentration of the compound of formula (I) during the dosing schedule is less than 10 ng/ml, less than 1 ng/ml, less than 0.1 ng/ml, less than 0.01 ng/ml, or less than 0.001 ng/ml.

In some embodiments, the level of the of the compound of formula (I) administered during the dosing schedule may be undetectable during some portion of the dosing holiday.

In some embodiments, the maximum serum concentration of the of the compound of formula (I) during the dosing schedule is higher during an initial phase of administration, and lower in subsequent phases.

In some embodiments, the maximum serum concentration of the compound of formula (I) during the initial (loading) phase of administration is less than 500 ng/ml, less than 400 ng/ml, less than 300 ng/ml, less than 200 ng/ml, less than 150 ng/ml, less than 120 ng/ml, less than 100 ng/ml, less than 90 ng/ml, less than 80 ng/ml, less than 70 ng/ml, less than 60 ng/ml, or less than 50 ng/ml.

In some such embodiments, the maximum serum concentration of the compound of formula (I) during the initial phase of administration is from 5 ng/ml to 250 ng/ml. In some embodiments, the maximum serum concentration of the compound of formula (I) during the subsequent (maintenance) phase of administration is less than 350 ng/ml, less than 200 ng/ml, less than 120 ng/ml, less than 100 ng/ml, less than 90 ng/ml, less than 80 ng/ml, less than 70 ng/ml, less than 60 ng/ml, or less than 50 ng/ml, less than 40 ng/ml, less than 35 ng/ml, or less than 10 ng/ml.

One of ordinary skill in the art will readily be aware of such methods as exist in the art for the monitoring of serum concentrations of pharmaceutical agents, and means of adjusting dosages of the compounds disclosed herein in order to achieve the desired serum concentrations. In some embodiments, the weekly dose to be administered is 600 mg or less. In some embodiments, the weekly dose is to be administered is 500 mg or less, 400 mg or less, 300 mg or less, 200 mg or less, 100 mg or less, 50 mg or less, 40 mg or less, 25 mg or less, 10 mg or less, or 5 mg or less, or within a range defined by any two of the foregoing.

According to the present application, the dosing schedule may be varied in order to attain the desired therapeutic effect. In particular, variations in the dosing schedule as described may be repeated throughout the duration of the treatment.

For example, in certain embodiments, the first dosage may be higher, lower, or the same as the dosages following the first dosage. In addition, a loading dose may precede the disclosed dosing regimen, and a dosing holiday may or may not follow the administration of the loading dose.

The methods described herein may utilize any of a variety of suitable forms for a variety of routes for administration, for example, for oral, nasal, rectal, topical (including transdermal), ocular, intracerebral, intracranial, intrathecal, intra-arterial, intravenous, intramuscular, or other parental routes of administration. The skilled artisan will appreciate that oral and nasal compositions include compositions that are administered by inhalation, and made using available methodologies. Depending upon the particular route of administration desired, a variety of pharmaceutically-acceptable carriers well-known in the art may be used. Pharmaceutically-acceptable carriers include, for example, solid or liquid fillers, diluents, hydrotropies, surface-active agents, and encapsulating substances. Optional pharmaceutically-active materials may be included, which do not substantially interfere with the activity of the compound. The amount of carrier employed in conjunction with the compound is sufficient to provide a practical quantity of material for administration per unit dose of the compound. Techniques and compositions for making dosage forms useful in the methods described herein are described in e.g., Modern Pharmaceutics, 4th Ed., Chapters 9 and 10 (Banker & Rhodes, editors, 2002); Lieberman et al., Pharmaceutical Dosage Forms: Tablets (1989); and Ansel, Introduction to Pharmaceutical Dosage Forms 8th Edition (2004).

Various oral dosage forms can be used, including such solid forms as tablets, capsules, granules and bulk powders. Tablets can be compressed, tablet triturates, enteric-coated, sugar-coated, film-coated, or multiple-compressed, containing suitable binders, lubricants, diluents, disintegrating agents, coloring agents, flavoring agents, flow-inducing agents, and melting agents. Liquid oral dosage forms include aqueous solutions, emulsions, suspensions, solutions and/or suspensions reconstituted from non-effervescent granules, and effervescent preparations reconstituted from effervescent granules, containing suitable solvents, preservatives, emulsifying agents, suspending agents, diluents, sweeteners, melting agents, coloring agents and flavoring agents.

Other formulations useful for systemic delivery of the active agent(s) include sublingual, buccal and nasal dosage forms. Such formulations typically comprise one or more of soluble filler substances, such as sucrose, sorbitol and mannitol; and binders, such as acacia, microcrystalline cellulose, carboxymethyl cellulose and hydroxypropyl methyl cellulose. Glidants, lubricants, sweeteners, colorants, antioxidants and flavoring agents disclosed above may also be included.

Preservatives that may be used in the pharmaceutical compositions disclosed herein include, but are not limited to, benzalkonium chloride, PHMB, chlorobutanol, thimerosal, phenylmercuric, acetate and phenylmercuric nitrate. A useful surfactant is, for example, Tween 80. Likewise, other useful vehicles used in the ophthalmic preparations disclosed herein may include, but are not limited to, polyvinyl alcohol, povidone, hydroxypropyl methyl cellulose, poloxamers, carboxymethyl cellulose, hydroxyethyl cellulose and purified water.

Tonicity adjustors may be added as needed or convenient. Tonicity adjustors include, but are not limited to, salts, particularly sodium chloride, potassium chloride, mannitol and glycerin, or any other suitable ophthalmically acceptable tonicity adjustor.

For intravenous administration, the compounds and compositions described herein may be dissolved or dispersed in a pharmaceutically acceptable diluent, such as a saline or dextrose solution. Suitable excipients may be included to achieve the desired pH, including but not limited to NaOH, sodium carbonate, sodium acetate, HCl, and citric acid. In various embodiments, the pH of the final composition ranges from 2 to 8, or preferably from 4 to 7. Antioxidant excipients may include sodium bisulfite, acetone sodium bisulfite, sodium formaldehyde, sulfoxylate, thiourea, and EDTA. Other non-limiting examples of suitable excipients found in the final intravenous composition may include sodium or potassium phosphates, citric acid, tartaric acid, gelatin, and carbohydrates, such as dextrose, mannitol, and dextran. Further acceptable excipients are described in Powell, et al., Compendium of Excipients for Parenteral Formulations, PDA J Pharm Sci and Tech 1998, 52 238-311 and Nema et al., Excipients and Their Role in Approved Injectable Products: Current Usage and Future Directions, PDA J Pharm Sci and Tech 2011, 65287-332. Antimicrobial agents, including but not limited to phenylmercuric nitrate, thimerosal, benzethonium chloride, benzalkonium chloride, phenol, cresol, and chlorobutanol may also be included to achieve a bacteriostatic or fungistatic solution.

The compositions for intravenous administration may be provided to caregivers in the form of one more solids that are reconstituted with a suitable diluent such as sterile water, saline or dextrose in water shortly prior to administration. In other embodiments, the compositions are provided in solution ready to administer parenterally. In still other embodiments, the compositions are provided in a solution that is further diluted prior to administration. In embodiments that include administering a combination of a compound described herein and another agent, the combination may be provided to caregivers as a mixture, or the caregivers may mix the two agents prior to administration, or the two agents may be administered separately.

The compound of formula (I) and/or the one or more additional therapeutic agents according to the methods of the present application described herein may be administered by oral, intravenous, intraarterial, intestinal, rectal, vaginal, nasal, pulmonary, topical, intradermal, transdermal, transbuccal, translingual, sublingual, or opthalmic administration, or any combination thereof.

When the compound of formula (I) is administered in combination with the one or more additional therapeutic agents, the one or more pharmaceutical agents may be administered simultaneously or sequentially. In some embodiments, the compound of formula (I) and/or the one or more additional therapeutic agents are administered by co-administration. The term “co-administration,” as used hereinafter, refers to any one of the following: simultaneous administration, sequential administration, overlapping administration, concomitant administration, interval administration, continuous administration, contemporaneous administration or any combination thereof. In some such embodiments of the method, sequential co-administration is carried out in any order.

In some embodiments, the compound of formula (I) and/or the one or more additional therapeutic agents are administered every other day for the duration of the treatment. In other embodiments, the compound of formula (I) and/or the one or more additional therapeutic agents are administered on two out of every three days for the duration of the treatment. In still other embodiments the compound of formula (I) and/or the one or more additional therapeutic agents are administered two out of every four days for the duration of the treatment.

In some embodiments, the compound of formula (I) and/or the one or more additional therapeutic agents are administered daily for one day, followed by a two day dosing holiday. In some embodiments, the compound of formula (I) and/or the one or more additional therapeutic agents are administered daily for one day, followed by a two day dosing holiday. In some embodiments, the compound of formula (I) and/or the one or more additional therapeutic agents are administered daily for one day, followed by a three day dosing holiday. In some embodiments, the compound of formula (I) and/or the one or more additional therapeutic agents are administered daily for one day, followed by a four day dosing holiday. In some embodiments, the compound of formula (I) and/or the one or more additional therapeutic agents are administered daily for one day, followed by a five day dosing holiday. In some embodiments, the compound of formula (I) and/or the one or more additional therapeutic agents are administered daily for one day, followed by a six day dosing holiday. In some embodiments, the compound of formula (I) and/or the one or more additional therapeutic agents are administered daily for one day, followed by a seven day dosing holiday. In some embodiments, the compound of formula (I) and/or the one or more additional therapeutic agents are administered daily for one day, followed by an eight day dosing holiday. In some embodiments, the compound of formula (I) and/or the one or more additional therapeutic agents are administered daily for one day, followed by a nine day dosing holiday. In some embodiments, the compound of formula (I) and/or the one or more additional therapeutic agents are administered daily for one day, followed by a ten day dosing holiday. In some embodiments, the compound of formula (I) and/or the one or more additional therapeutic agents are administered daily for one day, followed by an eleven day dosing holiday. In some embodiments, the compound of formula (I) and/or the one or more additional therapeutic agents are administered daily for one day, followed by a twelve day dosing holiday. In some embodiments, the compound of formula (I) and/or the one or more additional therapeutic agents are administered daily for one day, followed by a thirteen day dosing holiday. In some embodiments, the compound of formula (I) and/or the one or more additional therapeutic agents are administered daily for one day, followed by a fourteen day dosing holiday.

In some embodiments, the compound of formula (I) and/or the one or more additional therapeutic agents are administered daily for two days, followed by a one day dosing holiday. In some embodiments, the compound of formula (I) and/or the one or more additional therapeutic agents are administered daily for two days, followed by a two day dosing holiday. In some embodiments, dosages are administered daily for two days, followed by a three day dosing holiday. In some embodiments, the compound of formula (I) and/or the one or more additional therapeutic agents are administered daily for two days, followed by a four day dosing holiday. In some embodiments, the compound of formula (I) and/or the one or more additional therapeutic agents are administered daily for two days, followed by a five day dosing holiday. In some embodiments, the compound of formula (I) and/or the one or more additional therapeutic agents are administered daily for two days, followed by a six day dosing holiday. In some embodiments, the compound of formula (I) and/or the one or more additional therapeutic agents are administered daily for two days, followed by a seven day dosing holiday. In some embodiments, dosages are administered daily for two days, followed by an eight day dosing holiday. In some embodiments, the compound of formula (I) and/or the one or more additional therapeutic agents are administered daily for two days, followed by a nine day dosing holiday. In some embodiments, the compound of formula (I) and/or the one or more additional therapeutic agents are administered daily for two days, followed by a ten day dosing holiday. In some embodiments, the compound of formula (I) and/or the one or more additional therapeutic agents are administered daily for two days, followed by an eleven day dosing holiday. In some embodiments, the compound of formula (I) and/or the one or more additional therapeutic agents are administered daily for two days, followed by a twelve day dosing holiday. In some embodiments, the compound of formula (I) and/or the one or more additional therapeutic agents are administered daily for two days, followed by a thirteen day dosing holiday. In some embodiments, the compound of formula (I) and/or the one or more additional therapeutic agents are administered daily for two days, followed by a fourteen day dosing holiday.

In some embodiments, the compound of formula (I) and/or the one or more additional therapeutic agents are administered daily for three days, followed by a one day dosing holiday. In some embodiments, the compound of formula (I) and/or the one or more additional therapeutic agents are administered daily for three days, followed by a two day dosing holiday. In some embodiments, the compound of formula (I) and/or the one or more additional therapeutic agents are administered daily for three days, followed by a three day dosing holiday. In some embodiments, the compound of formula (I) and/or the one or more additional therapeutic agents are administered daily for three days, followed by a four day dosing holiday. In some embodiments, the compound of formula (I) and/or the one or more additional therapeutic agents are administered daily for three days, followed by a five day dosing holiday. In some embodiments, the compound of formula (I) and/or the one or more additional therapeutic agents are administered daily for three days, followed by a six day dosing holiday. In some embodiments, the compound of formula (I) and/or the one or more additional therapeutic agents are administered daily for three days, followed by a seven day dosing holiday. In some embodiments, the compound of formula (I) and/or the one or more additional therapeutic agents are administered daily for three days, followed by an eight day dosing holiday. In some embodiments, the compound of formula (I) and/or the one or more additional therapeutic agents are administered daily for three days, followed by a nine day dosing holiday. In some embodiments, the compound of formula (I) and/or the one or more additional therapeutic agents are administered daily for three days, followed by a ten day dosing holiday. In some embodiments, the compound of formula (I) and/or the one or more additional therapeutic agents are administered daily for three days, followed by an eleven day dosing holiday. In some embodiments, the compound of formula (I) and/or the one or more additional therapeutic agents are administered daily for three days, followed by a twelve day dosing holiday. In some embodiments, the compound of formula (I) and/or the one or more additional therapeutic agents are administered daily for three days, followed by a thirteen day dosing holiday. In some embodiments, the compound of formula (I) and/or the one or more additional therapeutic agents are administered daily for three days, followed by a fourteen day dosing holiday.

In some embodiments, the compound of formula (I) and/or the one or more additional therapeutic agents are administered daily for four days, followed by a one day dosing holiday. In some embodiments, the compound of formula (I) and/or the one or more additional therapeutic agents are administered daily for four days, followed by a two day dosing holiday. In some embodiments, the compound of formula (I) and/or the one or more additional therapeutic agents are administered daily for four days, followed by a three day dosing holiday. In some embodiments, the compound of formula (I) and/or the one or more additional therapeutic agents are administered daily for four days, followed by a four day dosing holiday. In some embodiments, the compound of formula (I) and/or the one or more additional therapeutic agents are administered daily for four days, followed by a five day dosing holiday. In some embodiments, the compound of formula (I) and/or the one or more additional therapeutic agents are administered daily for four days, followed by a six day dosing holiday. In some embodiments, the compound of formula (I) and/or the one or more additional therapeutic agents are administered daily for four days, followed by a seven day dosing holiday. In some embodiments, the compound of formula (I) and/or the one or more additional therapeutic agents are administered daily for four days, followed by an eight day dosing holiday. In some embodiments, the compound of formula (I) and/or the one or more additional therapeutic agents are administered daily for four days, followed by a nine day dosing holiday. In some embodiments, the compound of formula (I) and/or the one or more additional therapeutic agents are administered daily for four days, followed by a ten day dosing holiday. In some embodiments, the compound of formula (I) and/or the one or more additional therapeutic agents are administered daily for four days, followed by an eleven day dosing holiday. In some embodiments, the compound of formula (I) and/or the one or more additional therapeutic agents are administered daily for four days, followed by a twelve day dosing holiday. In some embodiments, the compound of formula (I) and/or the one or more additional therapeutic agents are administered daily for four days, followed by a thirteen day dosing holiday. In some embodiments, the compound of formula (I) and/or the one or more additional therapeutic agents are administered daily for four days, followed by a fourteen day dosing holiday.

In some embodiments, the compound of formula (I) and/or the one or more additional therapeutic agents are administered daily for five days, followed by a one day dosing holiday. In some embodiments, the compound of formula (I) and/or the one or more additional therapeutic agents are administered daily for five days, followed by a two day dosing holiday. In some embodiments, the compound of formula (I) and/or the one or more additional therapeutic agents are administered daily for five days, followed by a three day dosing holiday. In some embodiments, the compound of formula (I) and/or the one or more additional therapeutic agents are administered daily for five days, followed by a four day dosing holiday. In some embodiments, the compound of formula (I) and/or the one or more additional therapeutic agents are administered daily for five days, followed by a five day dosing holiday. In some embodiments, the compound of formula (I) and/or the one or more additional therapeutic agents are administered daily for five days, followed by a six day dosing holiday. In some embodiments, the compound of formula (I) and/or the one or more additional therapeutic agents are administered daily for five days, followed by a seven day dosing holiday. In some embodiments, the compound of formula (I) and/or the one or more additional therapeutic agents are administered daily for five days, followed by an eight day dosing holiday. In some embodiments, the compound of formula (I) and/or the one or more additional therapeutic agents are administered daily for five days, followed by a nine day dosing holiday. In some embodiments, the compound of formula (I) and/or the one or more additional therapeutic agents are administered daily for five days, followed by a ten day dosing holiday. In some embodiments, the compound of formula (I) and/or the one or more additional therapeutic agents are administered daily for five days, followed by an eleven day dosing holiday. In some embodiments, the compound of formula (I) and/or the one or more additional therapeutic agents are administered daily for five days, followed by a twelve day dosing holiday. In some embodiments, the compound of formula (I) and/or the one or more additional therapeutic agents are administered daily for five days, followed by a thirteen day dosing holiday. In some embodiments, the compound of formula (I) and/or the one or more additional therapeutic agents are administered daily for five days, followed by a fourteen day dosing holiday.

In some embodiments, the compound of formula (I) and/or the one or more additional therapeutic agents are administered daily for six days, followed by a one day dosing holiday. In some embodiments, the compound of formula (I) and/or the one or more additional therapeutic agents are administered daily for six days, followed by a two day dosing holiday. In some embodiments, the compound of formula (I) and/or the one or more additional therapeutic agents are administered daily for six days, followed by a three day dosing holiday. In some embodiments, the compound of formula (I) and/or the one or more additional therapeutic agents are administered daily for six days, followed by a four day dosing holiday. In some embodiments, the compound of formula (I) and/or the one or more additional therapeutic agents are administered daily for six days, followed by a five day dosing holiday. In some embodiments, the compound of formula (I) and/or the one or more additional therapeutic agents are administered daily for six days, followed by a six day dosing holiday. In some embodiments, the compound of formula (I) and/or the one or more additional therapeutic agents are administered daily for six days, followed by a seven day dosing holiday. In some embodiments, the compound of formula (I) and/or the one or more additional therapeutic agents are administered daily for six days, followed by an eight day dosing holiday. In some embodiments, the compound of formula (I) and/or the one or more additional therapeutic agents are administered daily for six days, followed by a nine day dosing holiday. In some embodiments, the compound of formula (I) and/or the one or more additional therapeutic agents are administered daily for six days, followed by a ten day dosing holiday. In some embodiments, the compound of formula (I) and/or the one or more additional therapeutic agents are administered daily for six days, followed by an eleven day dosing holiday. In some embodiments, the compound of formula (I) and/or the one or more additional therapeutic agents are administered daily for six days, followed by a twelve day dosing holiday. In some embodiments, the compound of formula (I) and/or the one or more additional therapeutic agents are administered daily for six days, followed by a thirteen day dosing holiday. In some embodiments, the compound of formula (I) and/or the one or more additional therapeutic agents are administered daily for six days, followed by a fourteen day dosing holiday.

In some embodiments, the compound of formula (I) and/or the one or more additional therapeutic agents are administered daily for seven days, followed by a one day dosing holiday. In some embodiments, the compound of formula (I) and/or the one or more additional therapeutic agents are administered daily for seven days, followed by a two day dosing holiday. In some embodiments, the compound of formula (I) and/or the one or more additional therapeutic agents are administered daily for seven days, followed by a three day dosing holiday. In some embodiments, the compound of formula (I) and/or the one or more additional therapeutic agents are administered daily for seven days, followed by a four day dosing holiday. In some embodiments, the compound of formula (I) and/or the one or more additional therapeutic agents are administered daily for seven days, followed by a five day dosing holiday. In some embodiments, the compound of formula (I) and/or the one or more additional therapeutic agents are administered daily for seven days, followed by a six day dosing holiday. In some embodiments, the compound of formula (I) and/or the one or more additional therapeutic agents are administered daily for seven days, followed by a seven day dosing holiday. In some embodiments, the compound of formula (I) and/or the one or more additional therapeutic agents are administered daily for seven days, followed by an eight day dosing holiday. In some embodiments, the compound of formula (I) and/or the one or more additional therapeutic agents are administered daily for seven days, followed by a nine day dosing holiday. In some embodiments, the compound of formula (I) and/or the one or more additional therapeutic agents are administered daily for seven days, followed by a ten day dosing holiday. In some embodiments, the compound of formula (I) and/or the one or more additional therapeutic agents are administered daily for seven days, followed by an eleven day dosing holiday. In some embodiments, dosage the compound of formula (I) and/or the one or more additional therapeutic agents es are administered daily for seven days, followed by a twelve day dosing holiday. In some embodiments, the compound of formula (I) and/or the one or more additional therapeutic agents are administered daily for seven days, followed by a thirteen day dosing holiday. In some embodiments, the compound of formula (I) and/or the one or more additional therapeutic agents are administered daily for seven days, followed by a fourteen day dosing holiday.

In some embodiments, the compound of formula (I) and/or the one or more additional therapeutic agents are administered daily for eight days, followed by a one day dosing holiday. In some embodiments, the compound of formula (I) and/or the one or more additional therapeutic agents are administered daily for eight days, followed by a two day dosing holiday. In some embodiments, the compound of formula (I) and/or the one or more additional therapeutic agents are administered daily for eight days, followed by a three day dosing holiday. In some embodiments, the compound of formula (I) and/or the one or more additional therapeutic agents are administered daily for eight days, followed by a four day dosing holiday. In some embodiments, the compound of formula (I) and/or the one or more additional therapeutic agents are administered daily for eight days, followed by a five day dosing holiday. In some embodiments, the compound of formula (I) and/or the one or more additional therapeutic agents are administered daily for eight days, followed by a six day dosing holiday. In some embodiments, the compound of formula (I) and/or the one or more additional therapeutic agents are administered daily for eight days, followed by a seven day dosing holiday. In some embodiments, the compound of formula (I) and/or the one or more additional therapeutic agents are administered daily for eight days, followed by an eight day dosing holiday. In some embodiments, the compound of formula (I) and/or the one or more additional therapeutic agents are administered daily for eight days, followed by a nine day dosing holiday. In some embodiments, the compound of formula (I) and/or the one or more additional therapeutic agents are administered daily for eight days, followed by a ten day dosing holiday. In some embodiments, the compound of formula (I) and/or the one or more additional therapeutic agents are administered daily for eight days, followed by an eleven day dosing holiday. In some embodiments, the compound of formula (I) and/or the one or more additional therapeutic agents are administered daily for eight days, followed by a twelve day dosing holiday. In some embodiments, the compound of formula (I) and/or the one or more additional therapeutic agents are administered daily for eight days, followed by a thirteen day dosing holiday. In some embodiments, the compound of formula (I) and/or the one or more additional therapeutic agents are administered daily for eight days, followed by a fourteen day dosing holiday.

In some embodiments, the compound of formula (I) and/or the one or more additional therapeutic agents are administered daily for nine days, followed by a one day dosing holiday. In some embodiments, the compound of formula (I) and/or the one or more additional therapeutic agents are administered daily for nine days, followed by a two day dosing holiday. In some embodiments, the compound of formula (I) and/or the one or more additional therapeutic agents are administered daily for nine days, followed by a three day dosing holiday. In some embodiments, the compound of formula (I) and/or the one or more additional therapeutic agents are administered daily for nine days, followed by a four day dosing holiday. In some embodiments, the compound of formula (I) and/or the one or more additional therapeutic agents are administered daily for nine days, followed by a five day dosing holiday. In some embodiments, the compound of formula (I) and/or the one or more additional therapeutic agents are administered daily for nine days, followed by a six day dosing holiday. In some embodiments, the compound of formula (I) and/or the one or more additional therapeutic agents are administered daily for nine days, followed by a seven day dosing holiday. In some embodiments, the compound of formula (I) and/or the one or more additional therapeutic agents are administered daily for nine days, followed by an eight day dosing holiday. In some embodiments, the compound of formula (I) and/or the one or more additional therapeutic agents are administered daily for nine days, followed by a nine day dosing holiday. In some embodiments, the compound of formula (I) and/or the one or more additional therapeutic agents are administered daily for nine days, followed by a ten day dosing holiday. In some embodiments, the compound of formula (I) and/or the one or more additional therapeutic agents are administered daily for nine days, followed by an eleven day dosing holiday. In some embodiments, the compound of formula (I) and/or the one or more additional therapeutic agents are administered daily for nine days, followed by a twelve day dosing holiday. In some embodiments, the compound of formula (I) and/or the one or more additional therapeutic agents are administered daily for nine days, followed by a thirteen day dosing holiday. In some embodiments, the compound of formula (I) and/or the one or more additional therapeutic agents are administered daily for nine days, followed by a fourteen day dosing holiday.

In some embodiments, the compound of formula (I) and/or the one or more additional therapeutic agents are administered daily for ten days, followed by a one day dosing holiday. In some embodiments, the compound of formula (I) and/or the one or more additional therapeutic agents are administered daily for ten days, followed by a two day dosing holiday. In some embodiments, the compound of formula (I) and/or the one or more additional therapeutic agents are administered daily for ten days, followed by a three day dosing holiday. In some embodiments, the compound of formula (I) and/or the one or more additional therapeutic agents are administered daily for ten days, followed by a four day dosing holiday. In some embodiments, the compound of formula (I) and/or the one or more additional therapeutic agents are administered daily for ten days, followed by a five day dosing holiday. In some embodiments, the compound of formula (I) and/or the one or more additional therapeutic agents are administered daily for ten days, followed by a six day dosing holiday. In some embodiments, the compound of formula (I) and/or the one or more additional therapeutic agents are administered daily for ten days, followed by a seven day dosing holiday. In some embodiments, the compound of formula (I) and/or the one or more additional therapeutic agents are administered daily for ten days, followed by an eight day dosing holiday. In some embodiments, the compound of formula (I) and/or the one or more additional therapeutic agents are administered daily for ten days, followed by a nine day dosing holiday. In some embodiments, the compound of formula (I) and/or the one or more additional therapeutic agents are administered daily for ten days, followed by a ten day dosing holiday. In some embodiments, the compound of formula (I) and/or the one or more additional therapeutic agents are administered daily for ten days, followed by an eleven day dosing holiday. In some embodiments, the compound of formula (I) and/or the one or more additional therapeutic agents are administered daily for ten days, followed by a twelve day dosing holiday. In some embodiments, the compound of formula (I) and/or the one or more additional therapeutic agents are administered daily for ten days, followed by a thirteen day dosing holiday. In some embodiments, the compound of formula (I) and/or the one or more additional therapeutic agents are administered daily for ten days, followed by a fourteen day dosing holiday.

In some embodiments, the compound of formula (I) and/or the one or more additional therapeutic agents are administered daily for eleven days, followed by a one day dosing holiday. In some embodiments, the compound of formula (I) and/or the one or more additional therapeutic agents are administered daily for eleven days, followed by a two day dosing holiday. In some embodiments, the compound of formula (I) and/or the one or more additional therapeutic agents are administered daily for eleven days, followed by a three day dosing holiday. In some embodiments, the compound of formula (I) and/or the one or more additional therapeutic agents are administered daily for eleven days, followed by a four day dosing holiday. In some embodiments, the compound of formula (I) and/or the one or more additional therapeutic agents are administered daily for eleven days, followed by a five day dosing holiday. In some embodiments, the compound of formula (I) and/or the one or more additional therapeutic agents are administered daily for eleven days, followed by a six day dosing holiday. In some embodiments, the compound of formula (I) and/or the one or more additional therapeutic agents are administered daily for eleven days, followed by a seven day dosing holiday. In some embodiments, the compound of formula (I) and/or the one or more additional therapeutic agents are administered daily for eleven days, followed by an eight day dosing holiday. In some embodiments, the compound of formula (I) and/or the one or more additional therapeutic agents are administered daily for eleven days, followed by a nine day dosing holiday. In some embodiments, the compound of formula (I) and/or the one or more additional therapeutic agents are administered daily for eleven days, followed by a ten day dosing holiday. In some embodiments, the compound of formula (I) and/or the one or more additional therapeutic agents are administered daily for eleven days, followed by an eleven day dosing holiday. In some embodiments, the compound of formula (I) and/or the one or more additional therapeutic agents are administered daily for eleven days, followed by a twelve day dosing holiday. In some embodiments, the compound of formula (I) and/or the one or more additional therapeutic agents are administered daily for eleven days, followed by a thirteen day dosing holiday. In some embodiments, the compound of formula (I) and/or the one or more additional therapeutic agents are administered daily for eleven days, followed by a fourteen day dosing holiday.

In some embodiments, the compound of formula (I) and/or the one or more additional therapeutic agents are administered daily for twelve days, followed by a one day dosing holiday. In some embodiments, the compound of formula (I) and/or the one or more additional therapeutic agents are administered daily for twelve days, followed by a two day dosing holiday. In some embodiments, the compound of formula (I) and/or the one or more additional therapeutic agents are administered daily for twelve days, followed by a three day dosing holiday. In some embodiments the compound of formula (I) and/or the one or more additional therapeutic agents are administered daily for twelve days, followed by a four day dosing holiday. In some embodiments, the compound of formula (I) and/or the one or more additional therapeutic agents are administered daily for twelve days, followed by a five day dosing holiday. In some embodiments, the compound of formula (I) and/or the one or more additional therapeutic agents are administered daily for twelve days, followed by a six day dosing holiday. In some embodiments, the compound of formula (I) and/or the one or more additional therapeutic agents are administered daily for twelve days, followed by a seven day dosing holiday. In some embodiments, the compound of formula (I) and/or the one or more additional therapeutic agents are administered daily for twelve days, followed by an eight day dosing holiday. In some embodiments, the compound of formula (I) and/or the one or more additional therapeutic agents are administered daily for twelve days, followed by a nine day dosing holiday. In some embodiments, the compound of formula (I) and/or the one or more additional therapeutic agents are administered daily for twelve days, followed by a ten day dosing holiday. In some embodiments, the compound of formula (I) and/or the one or more additional therapeutic agents are administered daily for twelve days, followed by an eleven day dosing holiday. In some embodiments, the compound of formula (I) and/or the one or more additional therapeutic agents are administered daily for twelve days, followed by a twelve day dosing holiday. In some embodiments, the compound of formula (I) and/or the one or more additional therapeutic agents are administered daily for twelve days, followed by a thirteen day dosing holiday. In some embodiments, the compound of formula (I) and/or the one or more additional therapeutic agents are administered daily for twelve days, followed by a fourteen day dosing holiday.

In some embodiments, the compound of formula (I) and/or the one or more additional therapeutic agents are administered daily for thirteen days, followed by a one day dosing holiday. In some embodiments, the compound of formula (I) and/or the one or more additional therapeutic agents are administered daily for thirteen days, followed by a two day dosing holiday. In some embodiments, the compound of formula (I) and/or the one or more additional therapeutic agents are administered daily for thirteen days, followed by a three day dosing holiday. In some embodiments, the compound of formula (I) and/or the one or more additional therapeutic agents are administered daily for thirteen days, followed by a four day dosing holiday. In some embodiments the compound of formula (I) and/or the one or more additional therapeutic agents are administered daily for thirteen days, followed by a five day dosing holiday. In some embodiments, the compound of formula (I) and/or the one or more additional therapeutic agents are administered daily for thirteen days, followed by a six day dosing holiday. In some embodiments, the compound of formula (I) and/or the one or more additional therapeutic agents are administered daily for thirteen days, followed by a seven day dosing holiday. In some embodiments, the compound of formula (I) and/or the one or more additional therapeutic agents are administered daily for thirteen days, followed by an eight day dosing holiday. In some embodiments, the compound of formula (I) and/or the one or more additional therapeutic agents are administered daily for thirteen days, followed by a nine day dosing holiday. In some embodiments, the compound of formula (I) and/or the one or more additional therapeutic agents are administered daily for thirteen days, followed by a ten day dosing holiday. In some embodiments, the compound of formula (I) and/or the one or more additional therapeutic agents are administered daily for thirteen days, followed by an eleven day dosing holiday. In some embodiments, the compound of formula (I) and/or the one or more additional therapeutic agents are administered daily for thirteen days, followed by a twelve day dosing holiday. In some embodiments, the compound of formula (I) and/or the one or more additional therapeutic agents are administered daily for thirteen days, followed by a thirteen day dosing holiday. In some embodiments, the compound of formula (I) and/or the one or more additional therapeutic agents are administered daily for thirteen days, followed by a fourteen day dosing holiday.

In some embodiments, the compound of formula (I) and/or the one or more additional therapeutic agents are administered daily for fourteen days, followed by a one day dosing holiday. In some embodiments, the compound of formula (I) and/or the one or more additional therapeutic agents are administered daily for fourteen days, followed by a two day dosing holiday. In some embodiments, the compound of formula (I) and/or the one or more additional therapeutic agents are administered daily for fourteen days, followed by a three day dosing holiday. In some embodiments the compound of formula (I) and/or the one or more additional therapeutic agents are administered daily for fourteen days, followed by a four day dosing holiday. In some embodiments the compound of formula (I) and/or the one or more additional therapeutic agents are administered daily for fourteen days, followed by a five day dosing holiday. In some embodiments, the compound of formula (I) and/or the one or more additional therapeutic agents are administered daily for fourteen days, followed by a six day dosing holiday. In some embodiments, the compound of formula (I) and/or the one or more additional therapeutic agents are administered daily for fourteen days, followed by a seven day dosing holiday. In some embodiments, the compound of formula (I) and/or the one or more additional therapeutic agents are administered daily for fourteen days, followed by an eight day dosing holiday. In some embodiments, the compound of formula (I) and/or the one or more additional therapeutic agents are administered daily for fourteen days, followed by a nine day dosing holiday. In some embodiments, the compound of formula (I) and/or the one or more additional therapeutic agents are administered daily for fourteen days, followed by a ten day dosing holiday. In some embodiments, the compound of formula (I) and/or the one or more additional therapeutic agents are administered daily for fourteen days, followed by an eleven day dosing holiday. In some embodiments, the compound of formula (I) and/or the one or more additional therapeutic agents are administered daily for fourteen days, followed by a twelve day dosing holiday. In some embodiments, the compound of formula (I) and/or the one or more additional therapeutic agents are administered daily for fourteen days, followed by a thirteen day dosing holiday. In some embodiments, the compound of formula (I) and/or the one or more additional therapeutic agents are administered daily for fourteen days, followed by a fourteen day dosing holiday.

In some embodiments, the compound of formula (I) and/or the one or more additional therapeutic agents are administered daily for thirty days followed by a thirty day dosing holiday. In some embodiments, the compound of formula (I) and/or the one or more additional therapeutic agents are administered daily for thirty days followed by a 25-30 day dosing holiday. In some embodiments, the compound of formula (I) and/or the one or more additional therapeutic agents are administered daily for thirty days followed by a 20-25 day dosing holiday. In some embodiments, the compound of formula (I) and/or the one or more additional therapeutic agents are administered daily for thirty days followed by a 15-20 day dosing holiday. In some embodiments, the compound of formula (I) and/or the one or more additional therapeutic agents are administered daily for thirty days followed by a 10-15 day dosing holiday. In some embodiments, the compound of formula (I) and/or the one or more additional therapeutic agents are administered daily for thirty days followed by a 5-10 day dosing holiday. In some embodiments, the compound of formula (I) and/or the one or more additional therapeutic agents are administered daily for thirty days followed by a 1-5 day dosing holiday.

In some embodiments, the compound of formula (I) and/or the one or more additional therapeutic agents are administered daily for 25-30 days followed by a thirty day dosing holiday. In some embodiments, the compound of formula (I) and/or the one or more additional therapeutic agents are administered daily for 25-30 days followed by a 25-30 day dosing holiday. In some embodiments, the compound of formula (I) and/or the one or more additional therapeutic agents are administered daily for 25-30 days followed by a 20-25 day dosing holiday. In some embodiments, the compound of formula (I) and/or the one or more additional therapeutic agents are administered daily for 25-30 days followed by a 15-20 day dosing holiday. In some embodiments, the compound of formula (I) and/or the one or more additional therapeutic agents s are administered daily for 25-30 days followed by a 10-15 dosing holiday. In some embodiments, the compound of formula (I) and/or the one or more additional therapeutic agents are administered daily for 25-30 days followed by a 5-10 day dosing holiday. In some embodiments, dosages are administered daily for 25-30 days followed by a 1-5 day dosing holiday.

In some embodiments, the compound of formula (I) and/or the one or more additional therapeutic agents are administered daily for 20-25 days followed by a thirty day dosing holiday. In some embodiments, the compound of formula (I) and/or the one or more additional therapeutic agents are administered daily for 20-25 days followed by a 25-30 day dosing holiday. In some embodiments, the compound of formula (I) and/or the one or more additional therapeutic agents are administered daily for 20-25 days followed by a 20-25 day dosing holiday. In some embodiments, the compound of formula (I) and/or the one or more additional therapeutic agents are administered daily for 20-25 days followed by a 15-20 day dosing holiday. In some embodiments, the compound of formula (I) and/or the one or more additional therapeutic agents are administered daily for 20-25 days followed by a 10-15 dosing holiday. In some embodiments, the compound of formula (I) and/or the one or more additional therapeutic agents are administered daily for 20-25 days followed by a 5-10 day dosing holiday. In some embodiments, the compound of formula (I) and/or the one or more additional therapeutic agents are administered daily for 20-25 days followed by a 1-5 day dosing holiday.

In some embodiments, the compound of formula (I) and/or the one or more additional therapeutic agents are administered daily for 15-20 days followed by a thirty day dosing holiday. In some embodiments, the compound of formula (I) and/or the one or more additional therapeutic agents are administered daily for 15-20 days followed by a 25-30 day dosing holiday. In some embodiments, the compound of formula (I) and/or the one or more additional therapeutic agents are administered daily for 15-20 days followed by a 20-25 day dosing holiday. In some embodiments, the compound of formula (I) and/or the one or more additional therapeutic agents are administered daily for 15-20 days followed by a 15-20 day dosing holiday. In some embodiments, the compound of formula (I) and/or the one or more additional therapeutic agents are administered daily for 15-20 days followed by a 10-15 day dosing holiday. In some embodiments, the compound of formula (I) and/or the one or more additional therapeutic agents are administered daily for 15-20 days followed by a 5-10 day dosing holiday. In some embodiments, the compound of formula (I) and/or the one or more additional therapeutic agents are administered daily for 15-20 days followed by a 1-5 day dosing holiday.

In any of the foregoing embodiments, the daily dosing may be administered in one dose administered once or day, or in two or more divided doses administered multiple times per day. For example, the compounds described herein may be administered once per day, twice per day, three times per day, or four times per day.

In some embodiments, the compound of formula (I) and the one or more additional therapeutic agents are administered in synergistically effective amount.

The present application is further illustrated by the following examples that should not be construed as limiting. The contents of all references, patents, and published patent applications cited throughout this application, as well as the Figures and Tables, are incorporated herein by reference.

### EXAMPLES

### Example 1, Preparation and Characterization of the Stabilizing Formulation of the Compound of Formula (I) (ASC 41)

### Preparation of Stabilizing Formulations of ASC41

1. Pretreatment of API and excipients: The API and excipients to be used for formulation study should be crushed, sieved and dried by conventional means of preparation technology to remove the lumps during storage and reduce the moisture content of easily hygroscopic excipients, so that they meet the standards for further preparation;
2. Compounding: API and excipients for granulation by wet method according to formulation ratio and preparation scale;
3. Mixing: Mix the API and excipients completed in the compounding by conventional means of preparation technology;
4. hot melt extrusion: Set the extrusion temperature according to different areas of the extruder; After preheating to the set temperature, keep the temperature for 15 min˜30 min, evenly add the mixed API and excipients in the form of manual feeding or weightlessness automatic feeder feeding, extrude at the preset extrusion speed; by adjusting the temperature, screw speed and feeding speed in different areas of the extruder cylinder, control the extrusion die temperature between 100° C. and 130° C., keep the screw torque within a stable range, and the material is transparent after extrusion; Adjust the extrusion speed and feeding speed, so that the retention time of the material in the hot melt extruder barrel is controlled within 30 min;
5. Crushing of extrudate: The cooled extrudate is crushed by conventional means of preparation technology;
6. Total mixing: According to the formulation ratio, add additional excipients, and mix the above materials by the conventional mixing means of preparation technology;
7. Preparation: The formulation b2 was compressed into 13 mm-6 mm (length*width) capsule tablets, and the hardness of the tablets was controlled at 70N˜130 N; the total mixture of formulation c2 was filled into VcapsPlus type 4 hydroxypropyl cellulose capsules.
8. Packaging: Fill the tablets of formulations b2 and the capsules of formulations c2 into high-density ethylene bottles, and seal with aluminum film;
9. Storage: Store the compounds shown in formula (I) tablets or capsules packaged in bottle at room temperature (not exceeding 30° C.)

### Test Example 1

### Formulation Composition:

TABLE 1
Formulation composition for Test Example 1

| Composition Formulation | Formulation Number | | | | | |
|---|---|---|---|---|---|---|
| (mg) | A1 | B1 | C1 | D1 | E1 | F1 |
| Formula(I) compound | 5 | 1 | 1 | 5 | 5 | 10 |
| Copovidone Kollidon VA64 | 0 | 45 | 40 | 0 | 165 | 0 |
| Copovidone Plasdone S-630 | 75 | 0 | 0 | 110 | 0 | 200 |
| Polyethylene glycol 6000 | 0 | 3 | 1 | 4 | 3.5 | 0 |
| Anhydrous Citric Acid | 0 | 0 | 0 | 0 | 1.5 | 0 |
| Colloidal silca dioxide | 0 | 1 | 0 | 1 | 0 | 0 |
| Extrudate Weight | 80 | 50 | 42 | 120 | 175 | 210 |
| Excipients | A1 | B1 | C1 | D1 | E1 | F1 |
| Calcium phosphate dibasic | 60 | 0 | 12 | 42 | 42 | 72 |
| Mannitol | 136 | 39 | 35 | 116 | 116 | 379 |
| Colloidal silca dioxide | 1.5 | 0.5 | 0.4 | 1.5 | 1 | 5 |
| Sodium Stearyl Fumarate | 2.5 | 0.5 | 0.6 | 2.5 | 3 | 4 |
| Total amount of excipients | 200 | 40 | 48 | 162 | 162 | 460 |
| Amounts | 280 | 90 | 90 | 282 | 337 | 670 |

### Process:

1. Pretreatment of API and excipients: The API and excipients to be used for formulation study should be crushed, sieved and dried by conventional means of preparation technology to remove the lumps during storage and reduce the moisture content of easily hygroscopic excipients, so that they meet the standards for further preparation;
2. Compounding: Weigh the API and excipients for hot melt extrusion according to the formulation ratio and preparation scale;
3. Mixing: mix the API and excipients completely in compounding by conventional means of preparation technology;
4. Hot melt extrusion: Set the extrusion temperature for different areas of the extruder, respectively; After preheating to the set temperature, keep the temperature for 15 min 30 min, evenly add the mixed API and excipients by manual feeding or weightlessness automatic feeder feeding, extrude at the preset extrusion speed; By adjusting the temperature, screw speed and feeding speed in different areas of the extruder barrel, control the extrusion die temperature between 100° C. and 130° C., keep the screw torque within a stable range, and the extruded material is transparent; Adjust the extrusion speed and feeding speed, so that the retention time of the material in the hot melt extruder barrel is controlled within 30 min;
5. Crushing of extrudate: The cooled extrudate is crushed by conventional means of preparation technology;
6. Total mixing: According to the formulation ratio, add additional excipients, and mix the above materials by the conventional mixing means of preparation technology;
7. Preparation: Compress the prescriptions A1, D1 and E1 into 13 mm×6 mm (length×width) capsule-shaped tablets, and control the hardness of the tablets at 70 N˜130 N. Formula F1 was compressed into 17.2 mm×8.1 mm (length×width) capsule-shaped tablets, and the hardness of the tablets was controlled between 90 N and 160 N. Fill the total blend of formulations B1 and C1 into Vcaps Plus No. 4 hydroxypropyl cellulose capsules;
8. Packaging: Fill the tablets of formulations A1, D1, E1 and F1 and the capsules of formulations B1 and C1 into high-density ethylene bottles, and seal with aluminum film;
9. Storage: Store the tablets or capsules of the compound shown in formula (I) in packaged bottles at room temperature (not exceeding 30° C.).

### Test Example 2

### Composition of Formulation:

TABLE 2
Composition of Example 2 Formulation

| | Formulation No. | | | | | |
|---|---|---|---|---|---|---|
| Composition (mg) | G1 | H1 | I1 | J1 | K1 | L1 |
| Compound of formula (I) | 5 | 5 | 5 | 5 | 5 | 5 |
| Copovidone Kollidon VA64 | 165 | 82.5 | 165 | 150 | 82.5 | 82.5 |
| Polyethylene glycol 6000 | 5 | 2.5 | 0 | 0 | 0 | 0 |
| Poloxamer 188 | 0 | 0 | 5 | 20 | 5 | 0 |
| Vitamin E polyethylene glycol | 0 | 0 | 0 | 0 | 0 | 5 |
| succinate (TPGS) | | | | | | |
| Weight of extrudate | 175 | 90 | 42 | 175 | 92.5 | 92.5 |

### Preparation Process:

1. Pretreatment of API and excipient materials: the API and excipient materials to be used in formulation research are crushed, sieved and dried by the conventional methods of preparation technology to remove caking during storage and reduce water content of hygroscopic excipient materials so as to meet the standards for further preparation;
2. Compounding: API and excipient materials for hot melt extrusion are weighed according to formulation proportion and preparation scale;
3. Mixing: API and excipient were mixed uniformly by conventional methods of preparation technology;
4. Hot melt extrusion: Setting the extrusion temperature for different areas of the extruder. After preheating to the set temperature, keep the temperature for 15 min-30 min, Add the uniformly mixed API and excipient by manual feeding or weight loss automatic feeder feeding uniformly, and extrude at preset extrusion speed. By adjusting the temperature of different areas of extruder barrel, screw rotation speed and feeding speed, the temperature of the extrusion die is controlled between 100° C. and 130° C., the screw torque is kept in a stable range, and the extruded material is transparent. Adjust the extrusion speed and feeding speed to control the residence time of materials in the barrel of hot melt extruder within 30 min;
5. Crushing the extrudate: Crushing the cooled extrudate by conventional means of preparation technology and passing through a 40-mesh sieve;

### Comparative Example 1

Prepared according to the a2 formulation in Table 2 and following preparation process

TABLE 3
Formulation composition for Comparative Example 1

| Composition Formulation(mg) | a2 |
|---|---|
| Granulation | / |
| Formula(I) compound | 5 |
| beta-cyclodextrin | 99 |
| Anhydrous Citric Acid | 0.5 |
| Colloidal silka dioxide | 0.5 |
| Total Wet Granulation | 105 |
| Excipients | / |
| Calcium phosphate dibasic | 45 |
| mannitol | 126 |
| Colloidal silca dioxide | 1.5 |
| Sodium Stearyl Fumarate | 2.5 |
| Total amount of excipients | 175 |
| Amounts | 280 |

### Preparation Process:

1. Pretreatment of API and excipients: The API and excipients to be used for formulation study should be crushed, sieved and dried by conventional means of preparation technology to remove the lumps during storage and reduce the moisture content of easily hygroscopic excipients, so that they meet the standards for further preparation;
2. Compounding: API and excipients for granulation by wet method according to formulation ratio and preparation scale;
3. Mixing: mix the API and excipients completed in compounding by conventional means of preparation technology;
4. Wet granulation: Use water as binder, evenly add it into the mixed granulation API and excipients, pass through a 24-mesh stainless steel screen for granulation, take the wet granules after granulation and dry them in a blast oven at 65° C. until the moisture content is less than 3% (rapid moisture determination by infrared weight loss at 105° C.).
5. Granulation: Granulate the dried granules by passing them through a 24-mesh stainless steel screen;
6. General mixing: According to the formulation ratio, add other excipients, and mix the above materials by conventional mixing means of preparation technology;
7. Preparation: compress the total mixed granules into 13 mm-6 mm (length*width) capsule tablets, and control the hardness of the tablets at 70 N-130 N;
8. Packaging: put the tablets of Formulation a2 into a high-density ethylene bottle and seal with aluminum film;
9. Storage: Store the tablets of the compound shown in formula (I) in packaged bottles at room temperature (not exceeding 30° C.) Comparative Example 2

### Formulation Composition:

TABLE 4
Formulation composition for Comparative Example 2

Formulation Number

| Composition Formulation(mg) | b2 | c2 |
|---|---|---|
| Formula (I) compound | 5 | 5 |
| Polyethylene caprolactam-polyvinyl | 110 | 0 |
| acetate-polyethylene glycol copolymer | | |
| Soluplus | | |
| Copovidone Kollidon VA64 | 0 | 55 |
| Polyethylene Glycol 6000 | 0 | 1.5 |
| Anhydrous citric acid | 0.5 | 0.5 |
| Colloidal silicon dioxide | 0.5 | 0 |
| Extrudate | 116 | 62 |
| Excipients | b2 | c2 |
| Anhydrous calcium hydrogen phosphate | 24 | 0 |
| Mannitol | 136 | 29 |
| Colloidal silicon dioxide | 1.5 | 0.5 |
| Sodium Stearyl Fumarate | 2.5 | 0.5 |
| Total amount of excipients | 164 | 30 |
| Total Amounts | 280 | 92 |

### Preparation Process:

1. Pretreatment of API and excipients: The API and excipients to be used for formulation study should be crushed, sieved and dried by conventional means of preparation technology to remove the lumps during storage and reduce the moisture content of easily hygroscopic excipients, so that they meet the standards for further preparation;
2. Compounding: API and excipients for granulation by wet method according to formulation ratio and preparation scale;
3. Mixing: Mix the API and excipients completed in the compounding by conventional means of preparation technology;
4. hot melt extrusion: Set the extrusion temperature according to different areas of the extruder; After preheating to the set temperature, keep the temperature for 15 min˜30 min, evenly add the mixed API and excipients in the form of manual feeding or weightlessness automatic feeder feeding, extrude at the preset extrusion speed; by adjusting the temperature, screw speed and feeding speed in different areas of the extruder cylinder, control the extrusion die temperature between 100° C. and 130° C., keep the screw torque within a stable range, and the material is transparent after extrusion; Adjust the extrusion speed and feeding speed, so that the retention time of the material in the hot melt extruder barrel is controlled within 30 min;
5. Crushing of extrudate: The cooled extrudate is crushed by conventional means of preparation technology;
6. Total mixing: According to the formulation ratio, add additional excipients, and mix the above materials by the conventional mixing means of preparation technology;
7. Preparation: The formulation b2 was compressed into 13 mm-6 mm (length*width) capsule tablets, and the hardness of the tablets was controlled at 70N˜130 N; the total mixture of formulation c2 was filled into VcapsPlus type 4 hydroxypropyl cellulose capsules.
8. Packaging: Fill the tablets of formulations b2 and the capsules of formulations c2 into high-density ethylene bottles, and seal with aluminum film;
9. Storage: Store the compounds shown in formula (I) tablets or capsules packaged in bottle at room temperature (not exceeding 30° C.)

### Comparative Example 3

It is prepared according to the E1 formulation (as shown in Table 5 below) in Example 1 of Chinese Invention Patent Application 202010105909.9 and the following preparation process.

TABLE 5
Formulation composition for Comparative Example 3

| Composition Formulation(mg) | d2 |
|---|---|
| Formula(I) compound | 5 |
| Polyethylene Glycol 1000 | 300 |
| Polyethylene Glycol 6000 | 100 |
| Poloxamer 188 | 90 |
| Anhydrous Citric Acid | 5 |
| Content Weight | 500 |
| Type of filled gelatin capsule | NO. 1 |

### Preparation Process:

1. Preparation of blank matrix: At 65° C., polyethylene glycol 1000, polyethylene glycol 4000, polyethylene glycol 6000, poloxamer 188, and anhydrous citric acid were successively added and stirred to completely melt;
2. De-bubbling: Standing to completely eliminate bubbles;
3. Addition of compounds shown in formula (I): Add the drug substance of compounds shown in formula (I) under stirring, and continue stirring to completely melt it into the matrix;
4. Filling capsule: Transfer the prepared molten contents to the preheated insulated cylinder of the capsule filling machine, enable the stirring function, fill the molten contents into the gelatin hard capsule with the preset filling parameters (control the average filling volume difference <2.5%, single capsule filling volume difference <5.0%), and cover the capsule cap;
5. Cooling: Lay flat at room temperature to quickly cool and solidify the contents;
6. Packaging: The capsule is loaded into a high-density ethylene bottle and sealed with aluminum film;
7. Storage: Store the formula (I) compound capsules packaged bottled at 2˜8° C.

### Comparative Example 4

Prepared according to e2 and f2 formulations in Table 6 and the following preparation process.

TABLE 6
Formulation Composition of Comparative Example 4

Formulation No.

| Formulation Composition (mg) | e2 | f2 |
|---|---|---|
| Compound of formula (I) | 5.0 | 5.0 |
| Copovidone Kollidon VA64 | 62.7 | 41.25 |
| Polyethylene glycol 6000 | 1.9 | 1.25 |
| Weight of extrudate | 69.6 | 47.5 |

### Preparation Process:

1. Pretreatment of API and excipient: the API and excipient to be used in formulation research are crushed, sieved and dried by the conventional means of preparation technology to remove caking during storage and reduce water content of hygroscopic excipient so as to meet the standards for further preparation;
2. Compounding: API and excipient for hot melt extrusion are weighed according to formulation proportion and preparation scale;
3. Mixing: the API and excipient were mixed uniformly by conventional means of preparation technology;
4. Hot melt extrusion: setting the extrusion temperature for different areas of the extruder; After preheating to the set temperature, keep the temperature for 15 min-30 min., add the uniformly mixed API and excipient in the way of manual feeding or weight loss automatic feeder feeding uniformly, and extrude at preset extrusion speed; By adjusting the temperature of different areas of the extruder barrel, screw rotation speed and feeding speed, the temperature of the extrusion die is controlled between 100° C. and 130° C., the screw torque is kept in a stable range, and the extruded material is transparent; Adjust the extrusion speed and feeding speed to control the residence time of materials in barrel of hot melt extruder within 30 min;
5. Crushing the extrudate: Crushing the cooled extrudate by conventional means of preparation technology and passing through a 40-mesh sieve;

### Comparative Example 5

Prepared according to g2 prescription in table 7 and the following preparation process.

TABLE 7
Formulation composition of Comparative Example 4

| Composition formulation (mg) | g2 |
|---|---|
| Compound represented by | 5 |
| formula (I) | |
| Copovidone Kollidon VA64 | 165 |
| Mannitol | 150 |
| Total | 320 |

### Preparation Process:

1. Pretreatment of API and excipient: API and excipient to be used in formulation research are crushed, sieved and dried by the conventional means of preparation technology to remove caking during storage and reduce the moisture content of hygroscopic excipient so as to meet the standards for further preparation;
2. Compounding: weighing API and excipient for dry granulation according to formulation proportion and preparation scale;
3. Mixing: mixing the API and excipient with finished ingredients evenly by the conventional means of preparation technology;
4. Dry granulation: roll the evenly mixed API and excipient under the pressure of 5.0 MPa and make them into thin slices.
5. Grading: grading by sieving with 24 mesh stainless steel sieve;
6. Packaging: packaging the granules obtained from Formulation e2 into double aluminum strips according to the dosage and sealing;
7. Preservation: the packaged tablets of the compound shown in formula (I) are stored at room temperature (not exceeding 30° C.).

### Effect Example 1

Take the granules obtained by grinding after thermal melting extrusion according to the formulations A1˜F1 of Test Example 1, grind the granules after wet granulation and drying according to the a2 formulation of Comparative Example 1, grind the granules obtained by grinding after thermal melting extrusion according to the b2, and c2 formulations of Comparative Example 2, and prepare the capsules according to the d2 formulation of Comparative Example 3, and compare the dissolution curves in water for 6 samples each.

Dissolution conditions: Take 900 mL of degassed water at 37° C.±0.5° C. as the dissolution medium, and perform 50 rpm for paddle method. The granules are directly and precisely weighed and then put in, and the capsules prepared according to the d2 formulation of Comparative Example 3 are put in the sedimentation basket for input. Take samples at 10, 20, 30, 45, 60, 90 and 120 min, respectively. Take the subsequent filtrate and dilute it with an equal proportion of 75% acetonitrile aqueous solution. Determine the concentration of the compound as shown in formula (I) by HPLC. Calculate the cumulative dissolution percentage of the compound as shown in formula (I) at different time points.

HPLC assay conditions: Select a chromatographic column packed with octadecylsilane bonded silica gel (Welch Ultimate® XB-C18 4.6*150 mm, 5 m, or equivalent chromatographic column) and 0.05% trifluoroacetic acid aqueous solution-acetonitrile (30:70) as mobile phase, flow rate 1.0 ml/min, column temperature 30° C., detection wavelength 230 nm. Accurately inject 20 μl of the reference solution and the test solution (50 μl of the 1 mg B1 and C1 formulation and 10 μl of the 10 mg F1 formulation) into the column respectively, record the chromatograms, and calculate the dissolution of each capsule with respect to the peak area by the external standard method.

### Results:

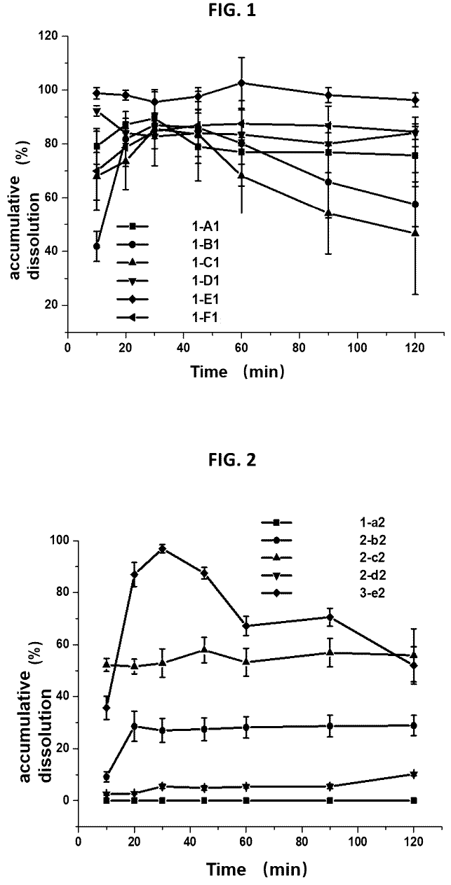

I. As shown in Table 8 and FIG. 1, with the formulation at the proportion of each embodiment of the present invention, the compounds shown in formula (I) can achieve the results of maximum dissolution >85%, which is similar to the results of the semisolid capsule of the Chinese invention patent application 202010105909.9 (compare the results of the d2 formulation of Comparative Example 3 in Table 9 and FIG. 2).
II. β-cyclodextrin is a common solubilizing excipient, and the dissolution of poorly soluble drugs can usually be improved to a certain extent after granulation with its wet method. However, the experimental results using the a2 formulation in example 1 showed that the solubility of the compounds shown in formula (I) was less than 1% at a higher proportion (1:19.8) of β-cyclodextrin dosage. This indicates that random application of common solubilization means does not necessarily improve the dissolution of the compounds shown in formula (I).
III. In the b2 formulation of Comparative Example 2, the polyethylene caprolactam-polyvinyl acetate-polyethylene glycol graft copolymer Soluplus, a popular hot melt extrusion excipient for solubilization, was mixed with the compound shown in formula (I) in a ratio of 22:1 and hot melt extrusion was performed, and the results showed a solubility of less than 1%. This suggests that the random selection of a hot melt extrusion excipient for solubilization does not necessarily have the effect of solubilizing the compounds shown in formula (I).
IV. In the b2 formulation of Comparative Example 2, the hot melt extrusion of copovidone Kollidon VA64 and the compound shown in formula (I) in a ratio of 1:11 had a maximum solubility of 57.9% within 2 hours, which was less than 85%. It can be seen that the proportion required for different excipients to achieve solubilization is also different.

### Conclusion

Simple application solubilization methods, such as β-cyclodextrin solubilization, are not suitable for increasing the dissolution of compounds shown in formula (I); simple application hot melt extrusion technology without screening materials, such as polyethylene caprolactam-polyvinyl acetate-polyethylene glycol copolymer Soluplus is not suitable for increasing the dissolution of compounds shown in formula (I); simple selection of high ratio excipients, such as copovidone Kollidon VA64 with the ratio of compounds shown in Formula (I) of 1:11, is not ideal. Therefore, only by selecting specific solubilizing materials and maintaining a reasonable ratio, can the compounds shown in formula (I) be satisfactorily solubilized.

TABLE 8
Aqueous Dissolution Results of Formulation Samples from Test Example 1(n = 6)

| Time | Dissolution Rate(Mean ± SD, %) | | | | | |
|---|---|---|---|---|---|---|
| min | A1 | B1 | C1 | D1 | E1 | F1 |
| 10 | 79.1 ± 6.6 | 41.9 ± 5.6 | 68.0 ± 8.8 | 92.3 ± 1.9 | 98.8 ± 2.1 | 70.0 ± 14.7 |
| 20 | 87.2 ± 2.3 | 81.8 ± 10.2 | 73.6 ± 10.5 | 84.1 ± 3.9 | 98.1 ± 1.8 | 78.7 ± 7.1 |
| 30 | 89.5 ± 2.0 | 87.0 ± 1.7 | 85.6 ± 13.7 | 82.8 ± 4.6 | 95.6 ± 4.5 | 84.7 ± 2.7 |
| 45 | 79.0 ± 6.2 | 86.1 ± 6.8 | 83.6 ± 17.3 | 84.0 ± 8.7 | 97.6 ± 1.9 | 86.9 ± 4.5 |
| 60 | 77.0 ± 4.5 | 80.2 ± 15.8 | 68.2 ± 16.2 | 83.5 ± 4.1 | 102.6 ± 9.4 | 87.5 ± 5.1 |
| 90 | 76.9 ± 10.6 | 65.9 ± 13.4 | 54.2 ± 15.1 | 80.1 ± 4.0 | 98.1 ± 2.8 | 86.8 ± 7.2 |
| 120 | 75.8 ± 11.6 | 57.6 ± 8.4 | 46.7 ± 22.7 | 84.1 ± 2.4 | 96.3 ± 2.6 | 84.5 ± 5.5 |

TABLE 9
Aqueous Dissolution Results of Formulation
Samples from Comparative Examples

| | |
|---|---|
| Time | Dissolution Rate(Mean ± SD, %) |

| min | a2 | b2 | c2 | d2 |
|---|---|---|---|---|
| 10 | <1.0 | 9.2 ± 2.0 | 52.2 ± 2.5 | 1.6 ± 0.4 |
| 20 | <1.0 | 28.6 ± 5.8 | 51.6 ± 2.8 | 2.6 ± 0.3 |
| 30 | <1.0 | 27.0 ± 4.6 | 52.9 ± 5.5 | 3.4 ± 0.8 |
| 45 | <1.0 | 27.5 ± 4.4 | 57.9 ± 4.9 | 3.9 ± 0.8 |
| 60 | <1.0 | 28.2 ± 4.1 | 53.2 ± 5.4 | 2.6 ± 0.6 |
| 90 | <1.0 | 28.7 ± 4.2 | 56.9 ± 5.5 | 4.8 ± 0.9 |
| 120 | <1.0 | 28.9 ± 3.9 | 55.9 ± 10.2 | 5.0 ± 0.6 |

### Effect Example 2

The pH of digestive juice in human gastrointestinal tract is increasing. Maintaining a high degree of supersaturation after oral administration is the prerequisite for insoluble drugs to be absorbed into systemic circulation to exert their efficacy. In this example, a simple dissolution test design in vitro (dissolution test of 2 h+4 h) was used to explain the reasons for choosing the composition ratio and preparation process of the present invention.

Take the granules obtained by hot melt extrusion according to G1-L1 formulation of Test Example 2, the granules obtained by hot melt extrusion according to g2-f2 formulation of Comparative Example 4, and the granules obtained by dry granulation according to g2 formulation of Comparative Example 5, and investigate the pH transition and supersaturation maintenance time of simulated human digestive juice.

The dissolution conditions were as follows: firstly, 750 mL degassed hydrochloric acid solution with pH 2.0 at 37° C.±0.5° C. was used as dissolution medium, and the dissolution was carried out with stirring at 50 rpm by paddle method for 2 hours, then degassed 250 mL 200 mM pH 6.8 phosphate buffer solution was added, and the dissolution was continued with stirring at 50 rpm by paddle method for 4 hours. Particles are directly and accurately weighed and then put in, and samples are taken at 15, 30, 45, 60, 90, 120, 180, 210, 240 and 360 min after putting in, and the subsequent filtrate is diluted with 75% acetonitrile water solution in equal proportion, and the concentration of the compound shown in formula (I) is determined by HPLC, and the formula (I) is calculated at different time points

The HPLC determination conditions were the same as those in Effect Example 1.

### Results:

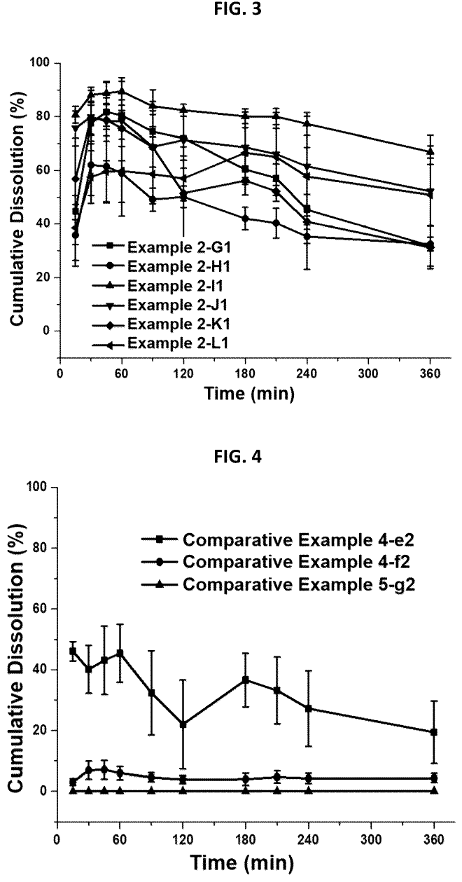

I. As shown in Table 10 and FIG. 3, the compound shown in formula (I) can achieve the highest dissolution rate of >60% and maintain dissolution rate of >30% at 6 h.
II. As shown in Table 11 and FIG. 4, when the proportion of copovidone is reduced to less than 15 parts, such as e2 and f2 in Comparative Example 4, when the proportion of copovidone is reduced to 12.54 parts and 8.25 parts respectively, the highest dissolution rate is only 46.1% and 7.1%, and the dissolution rate at 6 h is only 19.4% and 4.4%. This indicates that the ratio of copovidone is directly related to solubilization effect, and when the dosage is less than 15 parts, it is difficult to maintain supersaturated concentration at a higher degree.
III. In the g2 formulation of Comparative Example 5, the amount of copovidone is 33 parts, but because the dry granulation process is used instead of the hot melt extrusion, the results show that the dissolution within 6 hours is less than 1%. This indicates that the solubilization effect of the compound shown in formula (I) can be achieved only after hot melt extrusion, and the preparation process is very important for the implementation effect of the composition.

### Conclusion

The results of Effect Example 2 show once again that only by adopting a specific proportion of Copovidone and a specific hot melt extrusion preparation process can higher dissolution and longer supersaturation maintenance time be achieved.

TABLE 10
Dissolution results of formulation samples in Test Example 2 (n = 6)

| Time | Dissolution (Mean ± SD,%) | | | | | |
|---|---|---|---|---|---|---|
| Min | G1 | H1 | I1 | J1 | K1 | L1 |
| 15 | 44.7 ± 12.4 | 35.8 ± 11.5 | 80.7 ± 3.1 | 75.7 ± 6.5 | 56.8 ± 15.2 | 38.5 ± 12.1 |
| 30 | 77.6 ± 6.7 | 62.0 ± 11.4 | 88.1 ± 2.9 | 79.9 ± 9.9 | 79.4 ± 6.3 | 57.5 ± 9.8 |
| 45 | 81.7 ± 5.3 | 61.5 ± 13.7 | 88.8 ± 4.4 | 78.2 ± 14.6 | 78.8 ± 6.0 | 59.5 ± 11.3 |
| 60 | 80.5 ± 5.7 | 58.9 ± 16.7 | 89.4 ± 5.2 | 78.4 ± 14.8 | 75.6 ± 6.7 | 59.8 ± 11.8 |
| 90 | 74.4 ± 6.4 | 49.1 ± 4.3 | 83.9 ± 1.7 | 68.8 ± 21.3 | 68.7 ± 7.5 | 58.6 ± 13.8 |
| 120 | 72.0 ± 8.2 | 50.1 ± 4.0 | 82.4 ± 2.2 | 71.3 ± 10.5 | 51.5 ± 17.3 | 57.0 ± 8.4 |
| 180 | 60.3 ± 5.5 | 42.0 ± 4.2 | 80.1 ± 2.8 | 68.6 ± 13.1 | 56.2 ± 5.4 | 66.6 ± 9.3 |
| 210 | 57.2 ± 5.2 | 40.3 ± 5.6 | 80.0 ± 3.0 | 66.1 ± 15.5 | 52.2 ± 3.8 | 65.0 ± 10.6 |
| 240 | 45.3 ± 5.7 | 35.3 ± 2.6 | 77.3 ± 4.3 | 61.5 ± 18.6 | 40.8 ± 17.8 | 57.7 ± 10.9 |
| 360 | 31.8 ± 7.6 | 32.5 ± 2.6 | 66.9 ± 2.3 | 52.2 ± 20.9 | 31.2 ± 7.9 | 50.8 ± 11.4 |

TABLE 11
Dissolution results of formulation samples
in Comparative Examples 4-5 (n = 6)

| Time | Dissolution (Mean ± SD, %) | | |
|---|---|---|---|
| Min | e2 | f2 | g2 |
| 15 | 46.1 ± 3.2 | 3.1 ± 1.1 | <1.0 |
| 30 | 40.1 ± 7.9 | 6.9 ± 3.0 | <1.0 |
| 45 | 43.1 ± 11.3 | 7.1 ± 3.1 | <1.0 |
| 60 | 45.4 ± 9.6 | 6.0 ± 2.2 | <1.0 |
| 90 | 32.4 ± 13.9 | 4.6 ± 1.5 | <1.0 |
| 120 | 22.0 ± 14.6 | 3.9 ± 1.3 | <1.0 |
| 180 | 36.6 ± 8.8 | 4.0 ± 2.0 | <1.0 |
| 210 | 33.2 ± 11.0 | 4.7 ± 2.0 | <1.0 |
| 240 | 27.2 ± 12.4 | 4.3 ± 1.7 | <1.0 |
| 360 | 19.4 ± 10.3 | 4.4 ± 1.5 | <1.0 |

### Effect Example 3

Take capsules prepared according to the B1 formulation of Test Example 1 and tablets prepared according to the E1 formulation, respectively place them in high-density polyethylene bottles, seal with aluminum film, and then place them at 30° C.±2° C. with 65%±5% relative humidity for accelerated test. Take the capsules prepared according to the formulation d2 of Comparative Example 3, place them in a high-density polyethylene bottle, seal with aluminum film, and then place them at 25° C.±2° C. with relative humidity of 60%±10% for acceleration test. Related substances were determined for Group B1 capsules, Group E1 tablets and Group d2 capsules at the accelerated 1-month time point.

Determination of related substances: Using a column packed with octadecylsilane bonded silica gel (ACE UltraCore 2.5SuperC18 (4.6*150 mm) or equivalent) and 10 mM potassium dihydrogen phosphate aqueous solution as mobile phase A and acetonitrile as mobile phase B, perform gradient elution according to Table 12 (volume ratio); flow rate: 1.0 mL/min, detection wavelength: 278 nm, column temperature: 45° C.

TABLE 12
Volume ratio of mobile phases

| Time(min) | mobile phase A(%) | mobile phase B(%) |
|---|---|---|
| 0.00 | 80 | 20 |
| 0.50 | 80 | 20 |
| 8.00 | 45 | 55 |
| 15.00 | 45 | 55 |
| 25.00 | 30 | 70 |
| 50.00 | 15 | 85 |
| 50.10 | 80 | 20 |
| 55.00 | 80 | 20 |

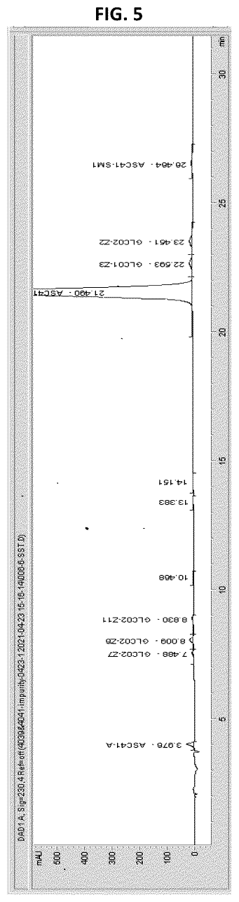

Take an appropriate amount of compounds and impurity reference substances as shown in formula (I), add acetonitrile to dissolve and dilute to produce a solution containing 0.5 mg of compounds and 0.001 mg of impurities per ml, as the system suitability test solution. Accurately inject 50 μl into the liquid chromatograph, and record the chromatogram. The resolution between the known impurities and the adjacent peaks should not be less than 1.5. Take 10 capsules, accurately weighed, pour the contents into a 100 ml volumetric flask, wash the inner wall of the capsule with acetonitrile for several times, and combine the washing liquid into the volumetric flask (for tablets, take 10 tablets, accurately weighed, grind into fine powder, accurately weigh an appropriate amount of tablet powder), dissolve with acetonitrile and prepare a solution containing 0.5 mg of the compound shown in formula (I) per ml as the solution; accurately measure 50 μl of the test solution, inject into the liquid chromatograph, and record the chromatogram. Calculate the sum of impurities and all impurities in compound capsules (or tablets) as shown in formula (I) by peak area normalization method. FIG. 5 shows an exemplary chromatogram of the impurities detected with the method described above.

### Results:

I. As shown in Table 13, for Test Example 1, the capsules and tablets prepared according to the formulations B1 and E1 were subjected to accelerated investigation at 30° C.±2° C. and 65%±5% relative humidity for 1 month, and the determination results of related substances showed that no significant change was found in all known individual impurities, unknown individual impurities and total impurities of the compounds shown in formula (I), especially the sum of GLC02-Z6 and GLC02-Z7 only increased by 0.02% and 0.04%, respectively. For the production batch, as shown in Table 14, for Test Example 1, the capsules and tablets prepared according to the formulations B1 and E1 were subjected to accelerated investigation at 30° C.±2° C. and 65%±5% relative humidity for 6 month, and the determination results of related substances showed that no significant change was found in all known individual impurities, unknown individual impurities and total impurities of the compounds shown in formula (I).
II. As shown in Table 13, for Comparative Example 3, after the capsules prepared according to d2 formulation were subjected to accelerated stability study at 25° C.±2° C. and 60%±10% relative humidity for 1 month, the determination results of related substances showed that the sum of GLC02-Z6 and GLC02-Z7 increased by 1.32%, the total impurities increased by 1.14%, and the related substances changed significantly. For the production batch, as shown in Table 14, for Comparative Example 3, after the capsules prepared according to d2 formulation were subjected to accelerated stability study at 25° C.±2° C. and 60%±10% relative humidity for 3 month, the determination results of related substances showed that the sum of GLC02-Z6 and GLC02-Z7 increased by 2.69%, the total impurities increased by 2.0%, and the related substances changed significantly.

TABLE 13
Effect of accelerated stability conditions on related substances in drug product

| | Related Substances(%) | | | | | |
|---|---|---|---|---|---|---|
| | Example 1 B1 | | Example 1 E1 | | Comparative Example 3 | |
| | Formulation Capsules | | Formulation Tablet | | d2 Formulation Capsules | |
| | | Accelerated | | Accelerated | | Accelerated |
| Impurities | 0 month | 1 month | 0 month | 1 month | 0 month | 1 month |
| A compound of formula (I)-SM1 | Not detected | Not detected | Not detected | Not detected | 0.04 | 0.04 |
| A compound of formula (I)-SM2 | Not detected | 0.02 | Not detected | 0.02 | Not detected | Not detected |
| A compound of formula (I)-A | 0.16 | 0.15 | 0.18 | 0.18 | 0.06 | 0.06 |
| GLC02-Z2 | Not detected | Not detected | 0.04 | 0.03 | 0.02 | 0.08 |
| GLC02-Z3 | 0.03 | 0.02 | 0.03 | Not detected | 0.08 | Not detected |
| GLC02-Z4 | Not detected | Not detected | Not detected | Not detected | Not detected | Not detected |
| GLC02-Z11 | 0.03 | 0.03 | 0.03 | 0.03 | 0.06 | 0.05 |
| Sum of GLC02-Z6 and GLC02-Z7 | 0.07 | 0.09 | 0.10 | 0.14 | 0.08 | 1.40 |
| Largest Single unknown impurity | 0.21 | 0.21 | 0.21 | 0.24 | 0.10 | 0.14 |
| Total impurities | 1.00 | 1.02 | 1.30 | 1.34 | 0.66 | 1.80 |

TABLE 14
Effect of different accelerated stability study on related substances in formulation for production batch

| | Related substance (%) | | | | | | | | | |
|---|---|---|---|---|---|---|---|---|---|---|
| | Example 1 | | | | | Example 2 | | | | |
| | B1 formulation capsule (production batch) | | | | | E1 formulation tablets (production batch) | | | | |
| Impurity | 0 | Accelerated | Accelerated | Accelerated | Accelerated | 0 | Accelerated | Accelerated | Accelerated | Accelerated |
| name | month | 1 month | 2 month | 3 month | 6 month | month | 1 month | 2 month | 3 month | 6 month |
| ASC41-SM1 | N.D. | N.D. | N.D. | N.D. | N.D. | N.D. | N.D. | N.D. | N.D. | N.D. |
| ASC41-SM2 | N.D. | N.D. | N.D. | N.D. | N.D. | N.D. | N.D. | N.D. | N.D. | N.D. |
| ASC41-A | 0.15 | 0.22 | 0.16 | 0.08 | 0.13 | 0.15 | 0.21 | 0.16 | 0.07 | 0.13 |
| GLC02-Z2 | 0.08 | 0.09 | 0.08 | 0.07 | 0.08 | 0.08 | 0.08 | 0.08 | 0.07 | 0.09 |
| GLC02-Z3 | N.D. | N.D. | N.D. | N.D. | N.D. | N.D. | N.D. | N.D. | N.D. | N.D. |
| GLC02-Z4 | N.D. | N.D. | N.D. | N.D. | N.D. | N.D. | N.D. | N.D. | N.D. | N.D. |
| GLC02-Z11 | 0.04 | 0.05 | 0.02 | 0.03 | 0.04 | 0.01 | 0.02 | 0.02 | 0.01 | 0.02 |
| Sum of | N.D. | N.D. | 0.05 | 0.04 | N.D. | N.D. | N.D. | 0.04 | 0.03 | N.D. |
| GLC02-Z6 | | | | | | | | | | |
| and GLC02- | | | | | | | | | | |
| Z7 | | | | | | | | | | |
| Other | 0.02 | N.D. | 0.02 | 0.04 | 0.06 | 0.01 | N.D. | 0.02 | 0.03 | 0.07 |
| largest | | | | | | | | | | |
| single | | | | | | | | | | |
| unknown | | | | | | | | | | |
| impurity | | | | | | | | | | |
| Total | 0.28 | 0.35 | 0.32 | 0.29 | 0.32 | 0.26 | 0.32 | 0.32 | 0.21 | 0.31 |
| impurity | | | | | | | | | | |

| | Related substance (%) | | | | |
|---|---|---|---|---|---|
| | Example 3 | | | | |
| | d2 formulation capsule (production batch) | | | | |
| Impurity | 0 | Accelerated | Accelerated | Accelerated | Accelerated |
| name | month | 1 month | 2 month | 3 month | 6 month |
| ASC41-SM1 | 0.06 | 0.06 | 0.06 | 0.06 | 0.06 |
| ASC41-SM2 | N.D. | N.D. | N.D. | N.D. | N.D. |
| ASC41-A | 0.02 | 0.02 | 0.03 | 0.03 | 0.02 |
| GLC02-Z2 | N.D. | 0.03 | 0.05 | 0.07 | 0.14 |
| GLC02-Z3 | 1.10 | 0.21 | 0.03 | N.D. | N.D. |
| GLC02-Z4 | N.D. | N.D. | N.D. | N.D. | N.D. |
| GLC02-Z11 | 0.03 | 0.05 | 0.07 | 0.07 | 0.08 |
| Sum of | 0.11 | 0.52 | 0.95 | 1.40 | 2.80 |
| GLC02-Z6 | | | | | |
| and GLC02- | | | | | |
| Z7 | | | | | |
| Other | 0.05 | 0.05 | 0.05 | 0.05 | 0.10 |
| largest | | | | | |
| single | | | | | |
| unknown | | | | | |
| impurity | | | | | |
| Total | 1.70 | 1.20 | 1.60 | 2.10 | 3.70 |
| impurity | | | | | |

### Conclusion

The accelerated results showed that the capsules or tablets of the compounds shown in formula (I) prepared according to the formulation of Test Example 1 had good results after accelerated stability study for 6 month at a temperature of 30° C.±2° C. and a relative humidity of 65%±5%, indicating that it has the prospect of long-term storage at room temperature.

The preliminary accelerated stability study results for 3 months under the conditions of temperature 25° C.±2° C. and relative humidity 60%±10% showed that the semi-solid capsule of the compound shown in formula (I) prepared according to the formulation of Comparative Example 3, the related substances, especially the sum of GLC02-Z6 and GLC02-Z7, changed significantly, which indicated that the formulation was only suitable for long-term use at 2° C.˜8° C., not suitable for long term storage at room temperature.

### Stabilization Formulation Impurities

Chromatographic conditions Octadecylsilane bonded silica gel was used as filler(YMC-Triart C18 250*4.6 mm S-5 m 12 nm or equivalent chromatographic column was recommended); the flow rate was 1.0 mL/min; the detection wavelength was 230 nm; the column temperature was 40° C.; the inject was 20 μL.
Mobile phase A: phosphate buffer:acetonitrile:tetrahydrofuran:butanol=690:150:80:80;
Mobile phase B: 0.03 mol/L potassium dihydrogen phosphate:acetonitrile:tetrahydrofuran:butanol=250:562:108:80
Phosphate buffer: 0.056 mol/L potassium dihydrogen phosphate: 0.003 mol/L dipotassium hydrogen phosphate=1:1

The gradient program is shown is Table 15 below:

TABLE 15
Gradient program

| Time(min) | A(%) | B(%) |
|---|---|---|
| 0.0 | 70 | 30 |
| 6.0 | 59 | 41 |
| 18.0 | 45 | 55 |
| 40.0 | 45 | 55 |
| 45.0 | 0 | 100 |
| 45.1 | 70 | 30 |
| 60.0 | 70 | 30 |

Accurately weigh about 6.25 mg of ASC41-SM1 and GLC02-Z11 reference substance, 18.75 mg of GLC02-Z3 reference substance, respectively, and put them into the same 50 mL volumetric flask; add acetonitrile solution to dissolve and dilute to the mark, shake well. Use it as the impurity reference stock solution 1.

Accurately weigh about 2.5 mg of GLC02-Z6, GLC02-Z7, ASC41-A and GLC02-Z2 reference substance, respectively, and put them into the same 10 mL volumetric flask; add acetonitrile solution to dissolve and dilute to the mark, shake well. Use it as the impurity reference stock solution 2.

Accurately weigh about 25 mg of ASC41 reference substance into a 50 mL volumetric flask, then pipette 1.0 mL of the impurity reference stock solution 1 and 2 to the above solution, add acetonitrile to dissolve and dilute to the mark, shake well. Use it as system suitability solution.

Take an appropriate amount of this product, fine powder, accurately weigh (about equivalent to 5 mg of ASC41), transfer it into a 10 mL volumetric flask, add an appropriate volume of acetonitrile solution, shake it well to mix, sonicate for 15 minutes, cool to room temperature, dilute to the mark with acetonitrile solution, shake well, filter, and take the filtrate as the test solution.

Determination method: Inject 20 μL each of the blank solution, system suitability solution and test solution into the liquid chromatograph, and record the chromatogram.

The blank solution chromatogram should have no interference at the retention time of the main peak.

The resolution between known impurities and their adjacent peaks in the system suitability solution should not be less than 1.0.

The results of each impurity were calculated by peak area normalization method with correction factor. Table 16 below shows the impurity limit, correction factor and relative retention time. FIG. 5 shows an exemplary chromatogram of the impurities detected with the method described above.

TABLE 16
Impurity characteristics

| | Relative | Relative | |
|---|---|---|---|
| Compound | Retention Time | Correction | Limit of |
| Name | min (RRT) | Factor | Impurities |
| ASC41-A | ~0.19 | 2.68 | ≤0.5% |
| GLC02-Z7 | ~0.35 | 1.19 | ≤0.5% |
| GLC02-Z6 | ~0.37 | 1.80 | ≤0.5% |
| GLC02-Z11 | ~0.41 | 0.56 | ≤0.5% |
| ASC41 | ~1.00 | NA | NA |
| GLC02-Z3 | ~1.05 | 1.27 | ≤1.5% |
| GLC02-Z2 | ~1.09 | 1.0 | ≤0.5% |
| ASC41-SM1 | ~1.24 | 1.0 | ≤0.5% |
| Unspecified impurity | NA | NA | ≤0.2% |
| Total Impurity | NA | NA | ≤3.0% |

### Example 2, Preparation of Fixed Dose Pharmaceutical Compositions Comprising ASC41 and One or More Additional Therapeutic Agents

Stable formulation of ASC41 is prepared by hot melt extrusion method described in Example 1. Granules or powders of the extruded ASC41 is mixed with one or more therapeutic agents, such as ASC40 (the compound of formula (II) or ASC42 (the compound of formula (III)) or a PPAR agonist, to form a mixture. The mixture is then pressed into fixed dose tablets or packed into fixed dose capsules.

### Example 3. Treatment of NASH with ASC41/ASC40 Combination or ASC41/ASC42 Combination in Rat Model

The effect of combination treatment with ASC41 and ASC40 is studied in a rat model based on the experimental design Shown in Table 17.

TABLE 17
Treatment of NASH in SD rats.

| | Study Title | | | | | |
|---|---|---|---|---|---|---|
| | Therapeutic efficacy of compounds in NASH induced by DEN + HFD − CHOL in SD rats | | | | | |
| | Animal species and genera | | | | | |
| | SD rats | | | Number of animals | 108 | |
| | | Number | | | | |
| Animal | | of | NASH | | Administration | Administration |
| Grouping | Group | Animal | modeling | Treatment | dose (mpk) | route |
| | Group-1 | 12 | No | Vehicle | Vehicle | |
| | Group-2 | 12 | Yes | Vehicle | Vehicle | |
| | Group-3 | 12 | Yes | ASC40L | 10 | PO |
| | Group-4 | 12 | Yes | ASC40H | 30 | PO |
| | Group-5 | 12 | Yes | ASC41 | 0.5 | PO |
| | Group-6 | 12 | Yes | ASC42 | 3 | PO |
| | Group-7 | 12 | Yes | ASC41 + ASC40L | 0.5 + 10 | PO |
| | Group-8 | 12 | Yes | ASC41 + ASC40H | 0.5 + 30 | PO |
| | Group-9 | 12 | Yes | ASC41 + ASC42 | 0.5 + 3 | PO |

| Model building | SD newborn rats are intraperitoneally injected with DEN two weeks after birth. The injected rats are nursed for a total | |
|---|---|---|
| | of four weeks. At four weeks, a total of 120 male rats with liver injury are selected and fed with high-fat and high- | |
| | cholesterol diet for 8 weeks. For normal control groups, 12 male rats are selected and no DEN is injected. This | |
| | group of rats are nursed for 4 weeks, then fed with standard maintenance diet. | |
| Test cycle | 4 Weeks lactation + 8 weeks model | |
| Administration cycle | After one week of the high-fat and high-cholesterol feed, treatments are given for seven weeks. | |
| Contents | 1. | Body weight: measure the weight twice a week from the day of modeling |
| | 2. | Fasting blood glucose: before cage separation, one week after high-fat feeding, and the end |
| | | point of the test |
| | 3. | Liver function test (fasting for 4 hours): the end point of the experiment; Serum ALT, AST, |
| | | TG, TC, HDL-c and LDL-c are determined |
| | 4. | Animals are euthanized at the end of experiment |

| | |
|---|---|
| a) | Liver is collected from each animal, weighted, photographed, and analyzed for TG and |
| | TC content, a portion of each liver is preserved at −80° for further analysis |
| b) | The remaining liver tissue is fixed with 10% formalin, and analyzed for liver pathology |
| | (NASH score is analyzed by HE staining, and liver fibrosis score is analyzed by SR |
| | staining) |

| | |
|---|---|
| 5. | Gene expression is analyzed by qPCR: |

| | |
|---|---|
| a) | FXR target genes: SREP-1c, SHP, FGF19 |
| b) | THR-b target genes: Cyp7a1, LDLR |
| c) | Fibrosis related genes: Col1a1, col3a1, MMP2 |

### Example 4. Treatment of NASH with ASC41/ASC4 Combination or ASC41/ASC42 Combination in Mouse Model

The effect of combination treatment with ASC41 and ASC40 is studied in a mouse model based on the experimental design shown in Table 18.

TABLE 18
Treatment of NASH in C57BL/6 mice

| | Study title: | | | | | |
|---|---|---|---|---|---|---|
| | Therapeutic efficacy of compounds in NASH induced by STZ + DEN + HFD in C57BL/6 mice | | | | | |
| | Animal species and genera | | | | | |
| | C57BL/6 mice | | | Number of animals | 84 | |
| | | | NASH | | Administration | Administration |
| Grouping | Group | Quantity | modeling | Medicines | dose (mpk) | mode |
| Group | Group-1 | 12 | No | Vehicle | Vehicle | |
| | Group-2 | 12 | Yes | Vehicle | Vehicle | |
| | Group-3 | 12 | Yes | ASC41 | 1 | PO QD |
| | Group 4 | 12 | Yes | ASC42 | 3 | PO QD |
| | Group-5 | 12 | Yes | Lanifibranor | 10 | PO QD |
| | Group-6 | 12 | Yes | ASC41 + ASC42 | 1 + 3 | PO QD |
| | Group-7 | 12 | Yes | ASC41 + Lanifibranor | 1 + 10 | PO QD |

| Model building | Neonatal C57BL/6 mice are injected with STZ subcutaneously. Two weeks after birth, DEN is | |
|---|---|---|
| | injected intraperitoneally once, and the mice are nursed for a total of four weeks (from birth). | |
| | 120 diabetic male mice are selected by detecting fasting blood glucose of mice. According to | |
| | animal weight and fasting blood glucose, the mice are randomly divided into 8 groups and fed | |
| | with 60% high fat diet for 8 weeks. For normal control group (Group 1), 12 male mice are selected | |
| | before STZ injection, nursed for four weeks, then fed with standard maintenance diet. | |
| Test cycle | 4 Weeks lactation + 8 weeks model | |
| Administration cycle | After the high-fat diet was given for a week, treatments were given seven weeks | |
| Contents | 1. | Body weight: measure the weight twice a week from the day of modeling |
| | 2. | Liver function test (fasting for 4 hours): the end point of the experiment; Serum |
| | | ALT, AST, TG, TC, HDL-c and LDL-c are determined |
| | 3. | Animals are euthanized at the end of experiment |

| | |
|---|---|
| a) | Liver is collected from each animal, weighted, photographed, and analyzed for TG and |
| | TC content, a portion of each liver is preserved at −80° for further analysis |
| b) | The remaining liver is fixed with 10% formalin, and the liver pathology is analyzed |
| | (NASH score is analyzed by HE staining, and liver fibrosis score is analyzed by SR |
| | staining) |

| | |
|---|---|
| 4. | Gene expression is detected by qPCR: |

| | |
|---|---|
| a) | FXR target genes: SREBP-1c, SHP, FGF19 |
| b) | THR-b target genes: Cyp7a1, LDLR |
| c) | Fibrosis related genes: Col1a1, col3a1, MMP2 |

### Example 5. Pharmacokinetics Studies in Mice

A pharmacokinetics study of ASC41, ASC42 and Lanifibranor is conducted in mice as shown in Table 19.

TABLE 19
Pharmacokinetics study of ASC41, ASC42 and Lanifibranor in mice

| | Experiment name | | | | |
|---|---|---|---|---|---|
| | Compound PK study | | | | |
| | Animal species and genera | | | | |
| | C57BL/6 mice | | Number of animals | 36 | |
| | | | | Administration | Administration |
| Experimental grouping | Group | Quantity | Medicines | dose (mpk) | mode |
| PK experiment | Group-1 | 6 | ASC41 low dose | 1 | PO |
| | Group-2 | 6 | ASC41 medium dose | 5 | PO |
| | Group-3 | 6 | ASC41 high dose | 20 | PO |
| | Group-4 | 6 | Lanifibranor | 10 | PO |
| | Group-5 | 6 | ASC41 low dose + | 1 + 3 | PO |
| | | | ASC42 | | |
| | Group-6 | 6 | ASC41 low dose + | 1 + 10 | PO |
| | | | Lanifibranor | | |

| | |
|---|---|
| Test cycle | 1 Day |
| Contents | After a single oral administration, blood samples were collected and analyzed at 0 (pre), |
| | 30 min, 2 h, 4 h, 8 h, 24 h and 24 h for the amount of ASC41, ASC42 and Lanifibranor. |

While various embodiments have been described above, it should be understood that such disclosures have been presented by way of example only and are not limiting. Thus, the breadth and scope of the subject compositions and methods should not be limited by any of the above-described exemplary embodiments, but should be defined only in accordance with the following claims and their equivalents.

The above description is for the purpose of teaching the person of ordinary skill in the art how to practice the present invention, and it is not intended to detail all those obvious modifications and variations of it which will become apparent to the skilled worker upon reading the description. It is intended, however, that all such obvious modifications and variations be included within the scope of the present invention, which is defined by the following claims. The claims are intended to cover the components and steps in any sequence which is effective to meet the objectives there intended, unless the context specifically indicates the contrary.

What is claimed is:

1. A pharmaceutical combination comprising:
(1) a compound of formula (I) formulated for storage at room temperature 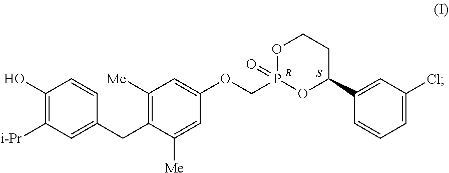
(2) at least one additional therapeutic agent;
and optionally one or more pharmaceutically acceptable carriers.

2. The pharmaceutical combination of claim 1, wherein the compound of formula (I) is formulated in a stabilizing formulation that allows the compound to be stored at room temperature for at least six months.

3. The pharmaceutical combination of claim 2, wherein the at least one additional therapeutic agent is a compound of formula (II):

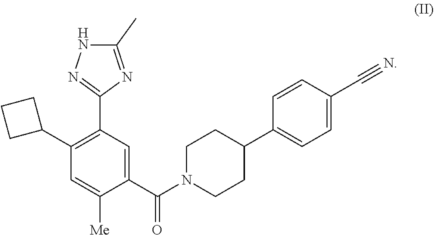 [SMILES: Cc1nc(-c2cc(C(=O)N3CCC(c4ccc(C#N)cc4)CC3)c(C)cc2C2CCC2)n[nH]1]

4. The pharmaceutical combination of claim 3, wherein the compound of formula (I) and the compound of formula (II) are formulated in a fixed dose tablet or capsule that contains 1-15 mg of the compound of formula (I) and 25-250 mg of the compound of formula (II).

5. The pharmaceutical combination of claim 4, wherein the fixed dose tablet or capsule contains 2.5 mg or 5 mg of the compound of formula (I) and 50 mg or 75 mg of the compound of formula (II).

6. The pharmaceutical combination of claim 4, wherein the fixed dose tablet contains 5 mg of the compound of formula (I) and 75 mg of the compound of formula (II).

7. The pharmaceutical combination of claim 2, wherein the at least one additional therapeutic agent is a compound of formula (III) or its pharmaceutically acceptable salts:

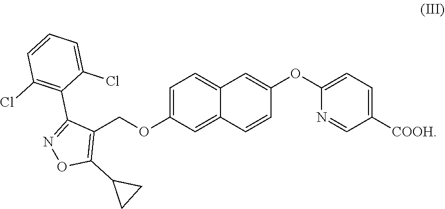 [SMILES: O=C(O)c1ccc(Oc2ccc3cc(OCc4c(-c5c(Cl)cccc5Cl)noc4C4CC4)ccc3c2)nc1]

8. The pharmaceutical combination of claim 7, wherein the compound of formula (I) and the compound of formula (III) are formulated in a fixed dose tablet or capsule that contains 1-15 mg of the compound of formula (I) and 5-100 mg of the sodium salt of the compound of formula (III).

9. The pharmaceutical combination of claim 8, wherein the fixed dose tablet or capsule contains 2.5 mg or 5 mg of the compound of formula (I), and 10 mg, 20 mg or 30 mg of the sodium salt of compound of formula (III).

10. The pharmaceutical combination of claim 1, wherein the at least one additional therapeutic agent is a PPAR agonist.

11. The pharmaceutical combination of claim 7, wherein the PPAR agonist is lanifibranor.

12. The pharmaceutical combination of claim 1, formulated for simultaneous, separate or sequential use in the treatment of non-alcoholic fatty liver disease (NAFLD) or nonalcoholic steatohepatitis (NASH).

13. The pharmaceutical combination of claim 1, formulated in an injectable suspension, a gel, an oil, a pill, a tablet, a suppository, a powder, a capsule, an aerosol, an ointment, a cream, a patch, or means of galenic forms for a prolonged and/or slow release.

14. A method for treatment of disease in a patient in need thereof, comprising:
administering to the patient an effective amount of the pharmaceutical combination of claim 1.

15. The method of claim 14, wherein the compound of formula (I) is formulated in a stabilizing formulation that allows the compound to be stored at room temperature for at least six months.

16. The method of claim 14, wherein the at least one additional therapeutic agent is a compound of formula (II):

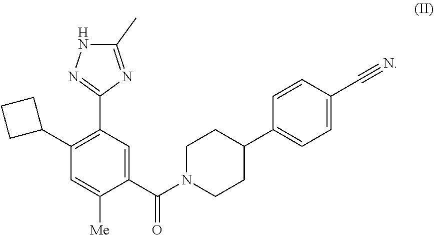 [SMILES: Cc1nc(-c2cc(C(=O)N3CCC(c4ccc(C#N)cc4)CC3)c(C)cc2C2CCC2)n[nH]1]

17. The method of claim 16, wherein the pharmaceutical combination is formulated as a fixed dose tablet or capsule that contains 1-15 mg of the compound of formula (I) and 25-250 mg of the compound of formula (II).

18. The method of claim 17, wherein the fixed dose tablet or capsule contains 2.5 mg or 5 mg of the compound of formula (I) and 50 mg of the compound of formula (II).

19. The method of claim 17, wherein the fixed dose tablet or capsule contains 2.5 mg or 5 mg of the compound of formula (I) and 75 mg of the compound of formula (II).

20. The method of claim 14, wherein the disease is selected from the group consisting of nonalcoholic fatty liver disease (NAFLD) and non-alcoholic steatohepatitis (NASH).
